(19) United States
(12) Patent Application Publication
(10) Pub. No.: US 2022/0131080 A1
(43) Pub. Date: Apr. 28, 2022

(54) CARBAZOLE DERIVATES FOR USE IN OPTOELECTRONIC DEVICES

(71) Applicant: CYNORA GMBH, Bruchsal (DE)

(72) Inventors: Michael Danz, Eggenstein-Leopoldshafen (DE); Damien Thirion, Karlsdorf-Neuthard (DE); Angela Digennaro, Heidelberg (DE); Stefan Seifermann, Buhl (DE); Damien Joly, Beinheim (DE)

(21) Appl. No.: 17/294,453

(22) PCT Filed: Nov. 8, 2019
(86) PCT No.: PCT/EP2019/080654

§ 371 Date: May 17, 2021

(30) Foreign Application Priority Data
Nov. 16, 2018 (EP) 18206790.0
Dec. 28, 2018 (EP) 18001020.9
Dec. 28, 2018 (EP) 18001022.5

(51) Int. Cl. H01L 51/00; C07D 403/14; C09K 11/06

(52) U.S. Cl. CPC H01L 51/0067; C07D 403/14; H01L 51/5016; C09K 11/06; C09K 2211/1018; H01L 51/0072

(57) ABSTRACT

The invention relates to an organic molecule, in particular for use in organic optoelectronic devices. According to the invention, the organic molecule has a structure of Formula I

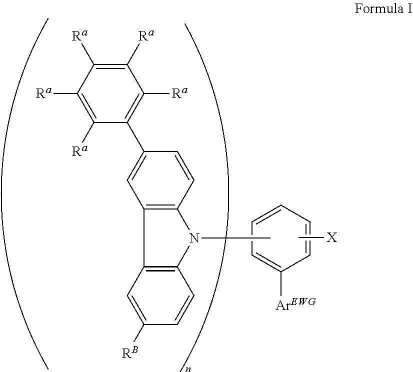

wherein
n is 1 or 2; and
X is selected from the group consisting of H, SiMe₃, SiPh₃, CN, and CF₃.

The invention relates to organic molecules and the use thereof in organic light-emitting diodes (OLEDs) and in other optoelectronic devices.

### DESCRIPTION

The object of the present invention is to provide molecules which are suitable for use in optoelectronic devices.

This object is achieved by the invention, which provides a new class of organic molecules.

According to the invention, the organic molecules are purely organic molecules, i.e., unlike metal complexes known for their use in optoelectronic devices, they do not contain metal ions.

According to the present invention, the organic molecules exhibit emission maxima in the blue, sky blue, or green spectral range. The organic molecules in particular exhibit emission maxima between 420 nm and 520 nm, preferably between 440 and 495 nm, more preferably between 450 nm and 470 nm. The photoluminescence quantum yields of the organic molecules according to the invention are in particular 70% and more. The molecules according to the invention in particular exhibit a thermally activated delayed fluorescence (TADF). The use of the molecules according to the invention in an optoelectronic device, for example an organic light-emitting diode (OLED), results in higher efficiencies of the device. The corresponding OLEDs have a higher stability than OLEDs having known emitter materials and comparable color.

The organic molecules according to the invention comprise or consist of a structure of Formula I

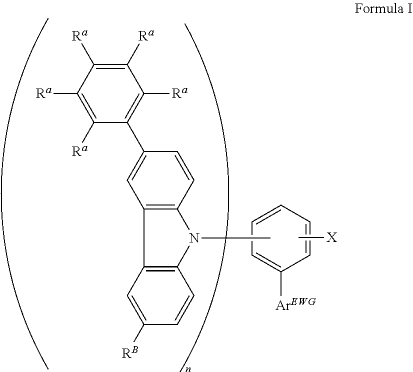

where

n is 1 or 2;

(so that for n=1, the following results:

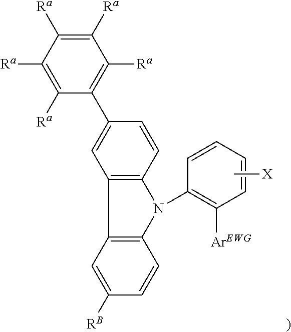

X is selected from the group consisting of H, SiMe₃, SiPh₃, CN, and CF₃;

Ar^{EWG} is selected from the group consisting of Formulas IIa to IIo

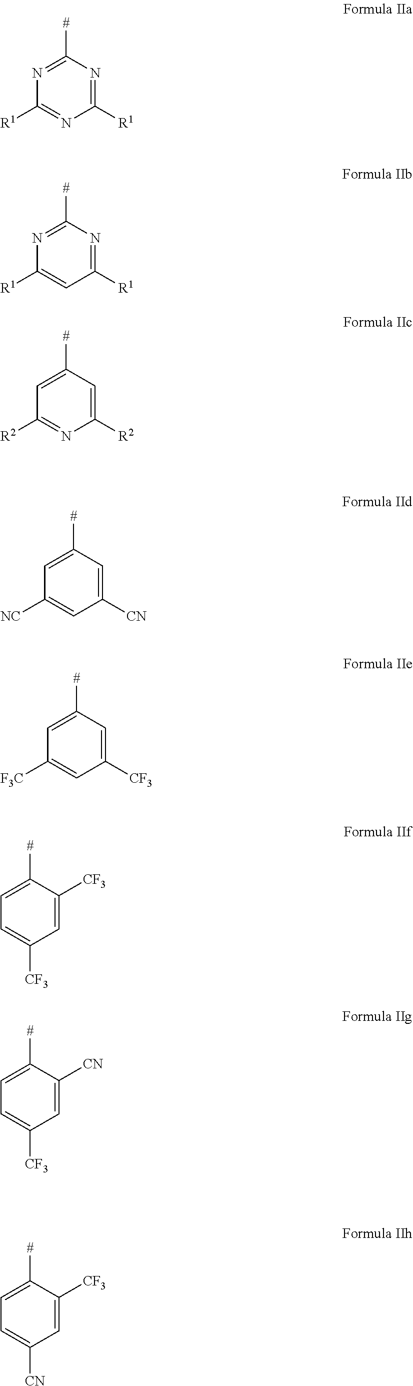

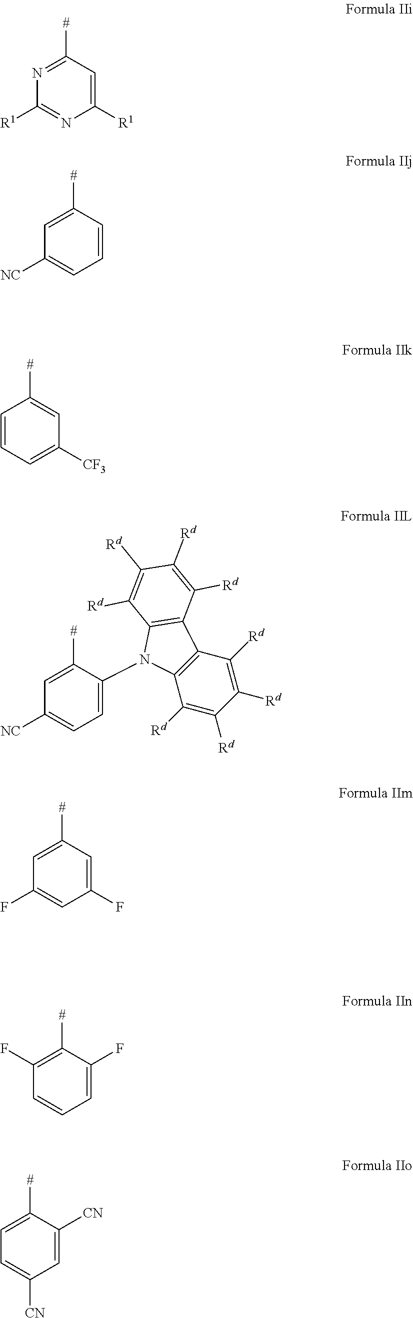

wherein # represents the binding site of a single bond, which connects Ar^{EWG} to the substituted central phenyl ring according to Formula I;

R¹ is selected independently of one another at each occurrence from the group consisting of hydrogen,

deuterium,

C₁-C₅ alkyl,
wherein one or more hydrogen atoms are optionally substituted by deuterium;

C₂-C₈ alkenyl,
wherein one or more hydrogen atoms are optionally substituted by deuterium;

C₂-C₈ alkynyl,
wherein one or more hydrogen atoms are optionally substituted by deuterium; and

C₈-C₁₈ aryl,
which is optionally substituted with one or more substituents R⁶;

R² is selected independently of one another at each occurrence from the group consisting of hydrogen,

deuterium,

C₁-C₅ alkyl,
wherein one or more hydrogen atoms are optionally substituted by deuterium;

C₂-C₈ alkenyl,
wherein one or more hydrogen atoms are optionally substituted by deuterium;

C₂-C₈ alkynyl,
wherein one or more hydrogen atoms are optionally substituted by deuterium; and

C₈-C₁₈ aryl,
which is optionally substituted with one or more substituents R⁶;

R^{d} is selected independently of one another at each occurrence from the group consisting of hydrogen, deuterium, N(R⁶)₂, OR⁶, Si(R⁶)₃, B(OR⁶)₂, OSO₂R⁶, CF₃, CN, F, Br, I,

C₁-C₄₀ alkyl,
which is optionally substituted with one or more substituents R⁶, and
wherein one or more non-adjacent CH₂ groups are optionally substituted by R⁶C═CR⁶, C≡C, Si(R⁶)₂, Ge(R⁶)₂, Sn(R⁶)₂, C═O, C═S, C═Se, C═NR⁶, P(═O)(R⁶), SO, SO₂, NR⁶, O, S, or CONR⁶;

C₁-C₄₀ alkoxy,
which is optionally substituted with one or more substituents R⁶, and
wherein one or more non-adjacent CH₂ groups are optionally substituted by R⁶C═CR⁶, C≡C, Si(R⁶)₂, Ge(R⁶)₂, Sn(R⁶)₂, C═O, C═S, C═Se, C═NR⁶, P(═O)(R⁶), SO, SO₂, NR⁶, O, S, or CONR⁶;

C₁-C₄₀ thioalkoxy,
which is optionally substituted with one or more substituents R⁶, and
wherein one or more non-adjacent CH₂ groups are optionally substituted by R⁶C═CR⁶, C≡C, Si(R⁶)₂, Ge(R⁶)₂, Sn(R⁶)₂, C═O, C═S, C═Se, C═NR⁶, P(═O)(R⁶), SO, SO₂, NR⁶, O, S, or CONR⁶;

C₂-C₄₀ alkenyl,
which is optionally substituted with one or more substituents R⁶, and
wherein one or more non-adjacent CH₂ groups are optionally substituted by R⁶C═CR⁶, C≡C, Si(R⁶)₂, Ge(R⁶)₂, Sn(R⁶)₂, C═O, C═S, C═Se, C═NR⁶, P(═O)(R⁶), SO, SO₂, NR⁶, O, S, or CONR⁶;

C₂-C₄₀ alkynyl,
which is optionally substituted with one or more substituents R⁶, and
wherein one or more non-adjacent CH₂ groups are optionally substituted by R⁶C═CR⁶, C≡C, Si(R⁶)₂, Ge(R⁶)₂, Sn(R⁶)₂, C═O, C═S, C═Se, C═NR⁶, P(═O)(R⁶), SO, SO₂, NR⁶, O, S, or CONR⁶;

C₆-C₆₀ aryl,
which is optionally substituted with one or more substituents R⁶; and

C₃-C₅₇ heteroaryl,
which is optionally substituted with one or more substituents R⁶;

R^{a} is selected independently of one another at each occurrence from the group consisting of R^{A},

hydrogen, deuterium, N(R⁶)₂, OR⁶, Si(R⁶)₃, B(OR⁶)₂, OSO₂R⁶, CF₃, CN, F, Br, I,

C₁-C₄₀ alkyl,
which is optionally substituted with one or more substituents R⁶, and
wherein one or more non-adjacent CH₂ groups are optionally substituted by R⁶C═CR⁶, C≡C, Si(R⁶)₂, Ge(R⁶)₂, Sn(R⁶)₂, C═O, C═S, C═Se, C═NR⁶, P(═O)(R⁶), SO, SO₂, NR⁶, O, S, or CONR⁶;

C₁-C₄₀ alkoxy,
which is optionally substituted with one or more substituents R⁶, and
wherein one or more non-adjacent CH₂ groups are optionally substituted by R⁶C═CR⁶, C≡C, Si(R⁶)₂, Ge(R⁶)₂, Sn(R⁶)₂, C═O, C═S, C═Se, C═NR⁶, P(═O)(R⁶), SO, SO₂, NR⁶, O, S, or CONR⁶;

C₁-C₄₀ thioalkoxy,
which is optionally substituted with one or more substituents R⁶, and
wherein one or more non-adjacent CH₂ groups are optionally substituted by R⁶C═CR⁶, C≡C, Si(R⁶)₂, Ge(R⁶)₂, Sn(R⁶)₂, C═O, C═S, C═Se, C═NR⁶, P(═O)(R⁶), SO, SO₂, NR⁶, O, S, or CONR⁶;

C₂-C₄₀ alkenyl,
which is optionally substituted with one or more substituents R⁶, and
wherein one or more non-adjacent CH₂ groups are optionally substituted by R⁶C═CR⁶, C≡C, Si(R⁶)₂, Ge(R⁶)₂, Sn(R⁶)₂, C═O, C═S, C═Se, C═NR⁶, P(═O)(R⁶), SO, SO₂, NR⁶, O, S, or CONR⁶;

C₂-C₄₀ alkynyl,
which is optionally substituted with one or more substituents R⁶, and
wherein one or more non-adjacent CH₂ groups are optionally substituted by R⁶C═CR⁶, C≡C, Si(R⁶)₂, Ge(R⁶)₂, Sn(R⁶)₂, C═O, C═S, C═Se, C═NR⁶, P(═O)(R⁶), SO, SO₂, NR⁶, O, S, or CONR⁶;

C₆-C₆₀ aryl,
which is optionally substituted with one or more substituents R⁶; and

C₃-C₅₇ heteroaryl,
which is optionally substituted with one or more substituents R⁶;

R⁶ is selected independently of one another at each occurrence from the group consisting of hydrogen, deuterium, OPh, CF₃, CN, F,

C₁-C₅ alkyl,
wherein one or more hydrogen atoms are optionally substituted independently of one another by deuterium, CN, CF₃, or F;

C₁-C₅ alkoxy,
wherein one or more hydrogen atoms are optionally substituted independently of one another by deuterium, CN, CF₃, or F;

C₁-C₅ thioalkoxy,
wherein one or more hydrogen atoms are optionally substituted independently of one another by deuterium, CN, CF₃, or F;

C₂-C₅ alkenyl,
wherein one or more hydrogen atoms are optionally substituted independently of one another by deuterium, CN, CF₃, or F;

C₂-C₅ alkynyl,
wherein one or more hydrogen atoms are optionally substituted independently of one another by deuterium, CN, CF₃, or F;

C₆-C₁₈ aryl,
which is optionally substituted with one or more C₁-C₅ alkyl substituents;

C₃-C₁₇ heteroaryl,
which is optionally substituted with one or more C₁-C₅ alkyl substituents;

N(C₆-C₁₈ aryl)₂;

N(C₃-C₁₇ heteroaryl)₂,

and N(C₃-C₁₇ heteroaryl)(C₆-C₁₈ aryl);

R^{A} is selected independently of one another at each occurrence from a chemical structure of Formula IIA

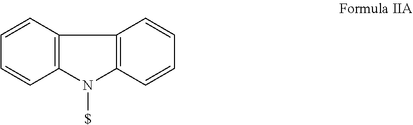

or a chemical structure of Formula IIB

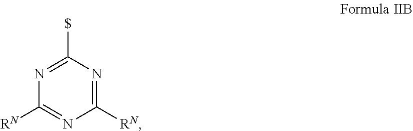

wherein R^{N} is selected from the group consisting of Me, ⁱPr, ^{t}Bu, CN, CF₃, and Ph;

wherein $ represents the binding site of a single bond by means of which R^{A} is connected.

wherein at least one substituent R^{a} is R^{A};

wherein R^{B} is selected from the group consisting of

H,

Ph,

CN,

CF₃,

of a chemical structure of Formula IIA-2

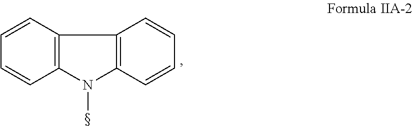

or a chemical structure of Formula IIB-2

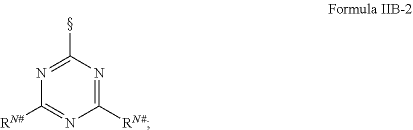

wherein R^{N#} is selected from the group consisting of Me, ⁱPr, ^{t}Bu, CN, CF₃, and Ph;

wherein § represents the binding site of a single bond.

In one embodiment of the invention, R¹ is independently of one another at each occurrence C₆-C₁₈ aryl, which is optionally substituted with one or more substituents R⁶.

In one embodiment of the invention, R¹ is selected independently of one another at each occurrence from the group consisting of

phenyl (Ph), which is optionally substituted with one or more substituents R⁶.

In one embodiment of the invention, R¹ is phenyl.

In one embodiment of the invention, R^{N} is phenyl.

In one embodiment of the invention, R^{N#} is phenyl.

In one embodiment of the invention, R² is selected independently of one another at each occurrence from the group consisting of

hydrogen,

CN,

CF₃, and

C₆-C₁₈ aryl, which is optionally substituted with one or more substituents R⁶.

In one embodiment of the invention, R² is selected independently of one another at each occurrence from the group consisting of

hydrogen,

CN,

CF₃, and

phenyl, which is optionally substituted with one or more substituents R⁶.

In one embodiment of the invention, R² is selected independently of one another at each occurrence from the group consisting of

hydrogen, CN, CF₃, and phenyl.

In one embodiment of the invention, R² is selected independently of one another at each occurrence from the group consisting of

hydrogen, CN, and CF₃.

In one embodiment of the invention, R² is selected independently of one another at each occurrence from the group consisting of

hydrogen and CN.

In one embodiment of the invention, R² is hydrogen at each occurrence.

In one embodiment of the organic molecules, R¹ is independently of one another at each occurrence

phenyl, which is optionally substituted with one or more substituents R⁶; and

R², which is selected independently of one another at each occurrence from the group consisting of

hydrogen, CN, CF₃, and phenyl, which is optionally substituted with one or more substituents R⁶.

In one embodiment of the organic molecules, R¹ is phenyl at each occurrence,

R² is selected independently of one another at each occurrence from the group consisting of

hydrogen, CN, CF₃, and Phenyl;

R^{N} is phenyl at each occurrence, and

R^{N#} is phenyl at each occurrence.

In one embodiment of the organic molecules, R¹ is phenyl at each occurrence,

R² is hydrogen at each occurrence,

R^{N} is phenyl at each occurrence; and

R^{N#} is phenyl at each occurrence.

In one embodiment, R⁶ is selected from the group consisting of Me, ⁱPr, ^{t}Bu, CN, CF₃, and Ph.

In one embodiment, X is selected from the group consisting of SiMe₃, SiPh₃, CN, and CF₃.

In a preferred embodiment, n is 1 and X is selected from the group consisting of SiMe₃, SiPh₃, CN, and CF₃.

In a preferred embodiment, n is 1 and X is selected from the group consisting of CN and CF₃.

In a preferred embodiment, n is 1 and X is CN.

In one embodiment, R^{B} is selected from the group consisting of

H,

Ph,

CN,

CF₃,

or a chemical structure of Formula IIB-2

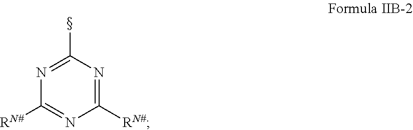

wherein R^{N#} is selected from the group consisting of Me, ⁱPr, ^{t}Bu, CN, CF₃, and Ph.

In one embodiment, R^{B} is selected from the group consisting of

H,

Ph,

CN,

CF₃,

or a chemical structure of Formula IIB-2

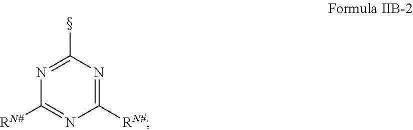

wherein R^{N#} is Ph.

In one embodiment, R^{B} is selected from the group consisting of

H,

Ph,

CN,

or a chemical structure of Formula IIB-2

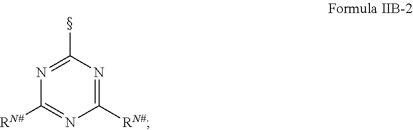

wherein R^{N#} is Ph.

In one embodiment, R^{B} is selected from the group consisting of

H,

or a chemical structure of Formula IIB-2

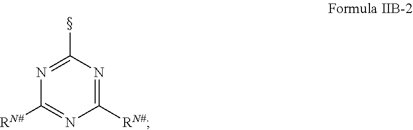

wherein R^{N#} is Ph.

In one embodiment, R^{B} is H.

In one embodiment, R^{B} consists of a chemical structure of Formula IIB-2

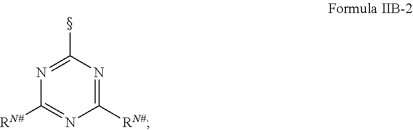

wherein R^{N#} is Ph.

In another preferred embodiment, n is 1 and R^{B} is selected from the group consisting of

H,

or a chemical structure of Formula IIB-2

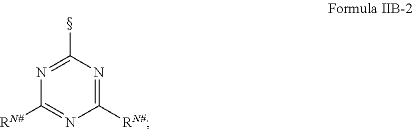

wherein R^{N#} is Ph.

In one embodiment of the organic molecules, exactly one substituent R^{a} is R^{A}.

In one embodiment, exactly two substituents R^{a} are, independently of one another, R^{A}, wherein R^{A} can be the same or different.

In another embodiment of the invention, R^{a} is selected independently of one another at each occurrence from the group consisting of

R^{A},

H,

D,

Me,

ⁱPr,

^{t}Bu,

CN,

CF₃,
Ph, which is optionally substituted with one or more substituents independently selected from the group consisting of Me, ⁱPr, ^{t}Bu, CN, CF₃, and Ph,
pyridinyl, which is optionally substituted with one or more substituents independently selected from the group consisting of Me, ⁱPr, ^{t}Bu, CN, CF₃, and Ph,
pyrimidinyl, which is optionally substituted with one or more substituents independently selected from the group consisting of Me, ⁱPr, ^{t}Bu, CN, CF₃, and Ph,
carbazolyl, which is optionally substituted with one or more substituents independently selected from the group consisting of Me, ⁱPr, ^{t}Bu, CN, CF₃, and Ph,
triazinyl, which is optionally substituted with one or more substituents independently selected from the group consisting of Me, ⁱPr, ^{t}Bu, CN, CF₃, and Ph,
and N(Ph)₂.

In another embodiment of the invention, R^{a} is selected independently of one another at each occurrence from the group consisting of

R^{A},

H,

D,

Me,

ⁱPr,

^{t}Bu,

CN,

CF₃,
Ph, which is optionally substituted with one or more substituents independently selected from the group consisting of Me, ⁱPr, ^{t}Bu, CN, CF₃, and Ph,
pyridinyl, which is optionally substituted with one or more substituents independently selected from the group consisting of Me, ⁱPr, ^{t}Bu, CN, CF₃, and Ph,
pyrimidinyl, which is optionally substituted with one or more substituents independently selected from the group consisting of Me, ⁱPr, ^{t}Bu, CN, CF₃, and Ph, and
triazinyl, which is optionally substituted with one or more substituents independently selected from the group consisting of Me, ⁱPr, ^{t}Bu, CN, CF₃, and Ph.

In another embodiment of the invention R^{a} is selected independently of one another at each occurrence from the group consisting of

R^{A},

H,

D,

Me,

ⁱPr,

^{t}Bu,

CN,

CF₃,
Ph, which is optionally substituted with one or more substituents independently selected from the group consisting of Me, ⁱPr, ^{t}Bu, CN, CF₃, and Ph, and
triazinyl, which is optionally substituted with one or more substituents independently selected from the group consisting of Me, ⁱPr, ^{t}Bu, CN, CF₃, and Ph.

In another embodiment of the invention, R^{a} is selected independently of one another at each occurrence from the group consisting of

R^{A}, H, D, CN, and CF₃.

In another embodiment of the invention, R^{a} is selected independently of one another at each occurrence from the group consisting of

R^{A}, H, and CN.

In another embodiment, the organic molecules according to the invention comprise or consist of a structure which is selected from the group consisting of Formula IIa, Formula IIa-2, and Formula IIa-3:

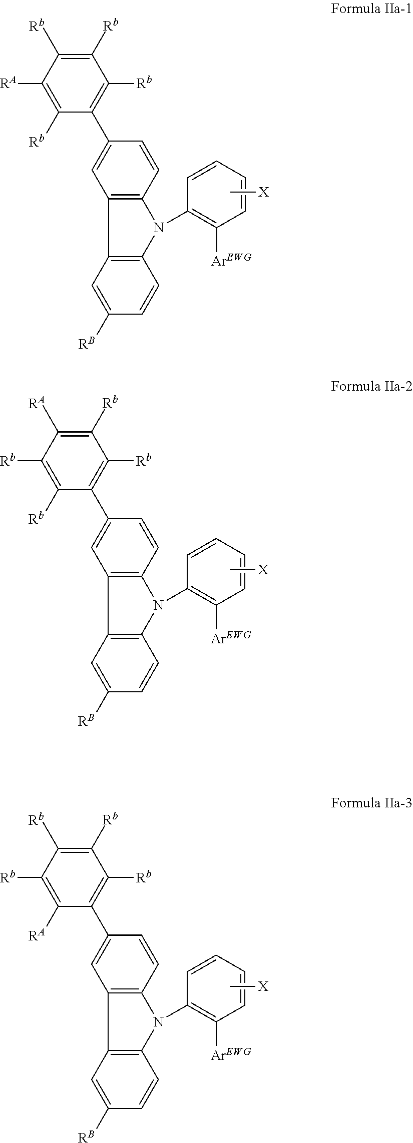

wherein

R^{b} is selected independently of one another at each occurrence from the group consisting of

R^{A},

hydrogen, deuterium, N(R⁶)₂, OR⁶, Si(R⁶)₃, B(OR⁶)₂, OSO₂R⁶, CF₃, CN, F, Br, I,

C₁-C₄₀ alkyl,
which is optionally substituted with one or more substituents R⁶, and
wherein one or more non-adjacent CH₂ groups are optionally substituted by R⁶C═CR⁶, C≡C, Si(R⁶)₂, Ge(R⁶)₂, Sn(R⁶)₂, C═O, C═S, C═Se, C═NR⁶, P(═O)(R⁶), SO, SO₂, NR⁶, O, S, or CONR⁶;

C₁-C₄₀ alkoxy,
which is optionally substituted with one or more substituents R⁶, and
wherein one or more non-adjacent CH₂ groups are optionally substituted by R⁶C═CR⁶, C≡C, Si(R⁶)₂, Ge(R⁶)₂, Sn(R⁶)₂, C═O, C═S, C═Se, C═NR⁶, P(═O)(R⁶), SO, SO₂, NR⁶, O, S, or CONR⁶;

C₁-C₄₀ thioalkoxy,
which is optionally substituted with one or more substituents R⁶, and
wherein one or more non-adjacent CH₂ groups are optionally substituted by R⁶C═CR⁶, C≡C, Si(R⁶)₂, Ge(R⁶)₂, Sn(R⁶)₂, C═O, C═S, C═Se, C═NR⁶, P(═O)(R⁶), SO, SO₂, NR⁶, O, S, or CONR⁶;

C₂-C₄₀ alkenyl,
which is optionally substituted with one or more substituents R⁶, and
wherein one or more non-adjacent CH₂ groups are optionally substituted by R⁶C═CR⁶, C≡C, Si(R⁶)₂, Ge(R⁶)₂, Sn(R⁶)₂, C═O, C═S, C═Se, C═NR⁶, P(═O)(R⁶), SO, SO₂, NR⁶, O, S, or CONR⁶;

C₂-C₄₀ alkynyl,
which is optionally substituted with one or more substituents R⁶, and
wherein one or more non-adjacent CH₂ groups are optionally substituted by R⁶C═CR⁶, C≡C, Si(R⁶)₂, Ge(R⁶)₂, Sn(R⁶)₂, C═O, C═S, C═Se, C═NR⁶, P(═O)(R⁶), SO, SO₂, NR⁶, O, S, or CONR⁶;

C₆-C₆₀ aryl,
which is optionally substituted with one or more substituents R⁶; and

C₃-C₅₇ heteroaryl,
which is optionally substituted with one or more substituents R⁶; and

aside from that, the aforementioned definitions apply.

In another embodiment, the organic molecules according to the invention comprise or consist of a structure which is selected from the group consisting of Formula IIa-1, Formula IIa-2, and Formula IIa-3:

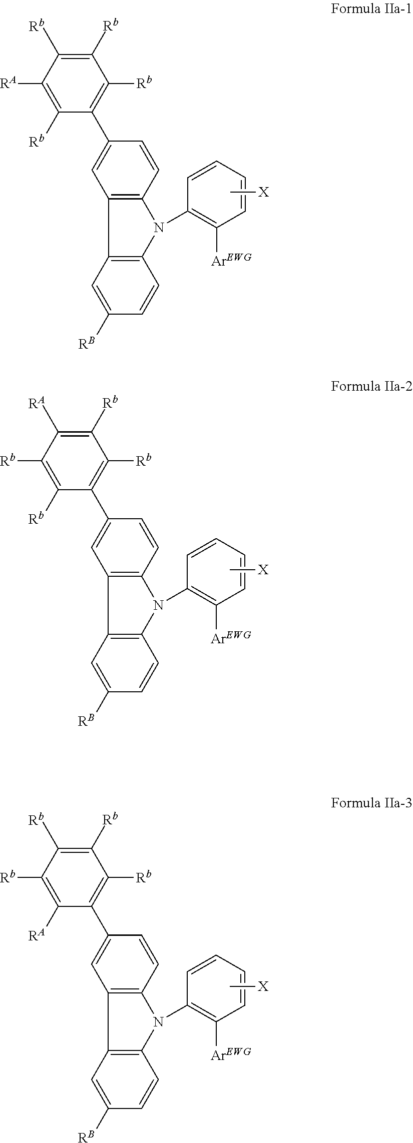

wherein R^{b} is selected independently of one another at each occurrence from the group consisting of

H,

Me,

ⁱPr,

^{t}Bu,

CN,

CF₃,
Ph, which is optionally substituted with one or more substituents independently selected from the group consisting of Me, ⁱPr, ^{t}Bu, CN, CF₃, and Ph,
pyridinyl, which is optionally substituted with one or more substituents independently selected from the group consisting of Me, ⁱPr, ^{t}Bu, CN, CF₃, and Ph,
pyrimidinyl, which is optionally substituted with one or more substituents independently selected from the group consisting of Me, ⁱPr, ^{t}Bu, CN, CF₃, and Ph,
carbazolyl, which is optionally substituted with one or more substituents independently selected from the group consisting of Me, ⁱPr, ^{t}Bu, CN, CF₃, and Ph,
triazinyl, which is optionally substituted with one or more substituents independently selected from the group consisting of Me, ⁱPr, ^{t}Bu, CN, CF₃, and Ph,

and N(Ph)₂.

In another embodiment of the invention, R^{b} is selected independently of one another at each occurrence from the group consisting of

H,

Me,

ⁱPr,

^{t}Bu,

CN,

CF₃,
Ph, which is optionally substituted with one or more substituents independently selected from the group consisting of Me, ⁱPr, ^{t}Bu, CN, CF₃, and Ph,
pyridinyl, which is optionally substituted with one or more substituents independently selected from the group consisting of Me, ⁱPr, ^{t}Bu, CN, CF₃, and Ph,
pyrimidinyl, which is optionally substituted with one or more substituents independently selected from the group consisting of Me, ⁱPr, ^{t}Bu, CN, CF₃, and Ph, and
triazinyl, which is optionally substituted with one or more substituents independently selected from the group consisting of Me, ⁱPr, ^{t}Bu, CN, CF₃, and Ph.

In another embodiment of the invention, R^{b} is selected independently of one another at each occurrence from the group consisting of

H,

CN,

CF₃.

In another embodiment of the invention, R^{b} is selected independently of one another at each occurrence from the group consisting of H and CN.

In a further embodiment of the invention, the organic molecules according to the invention comprise or consist of a structure which is selected from the group consisting of Formula IIc-1, Formula IIc-2, Formula IIc-3, Formula IIc-4, and Formula IIc-5:

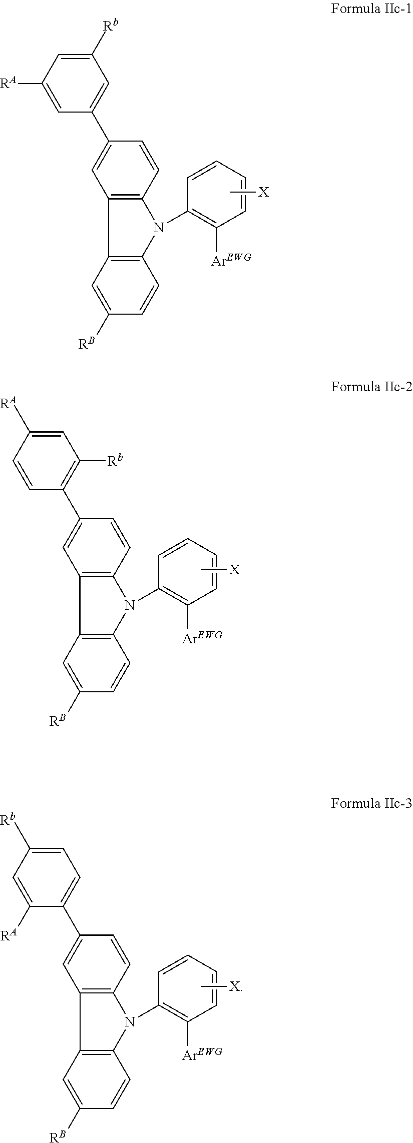

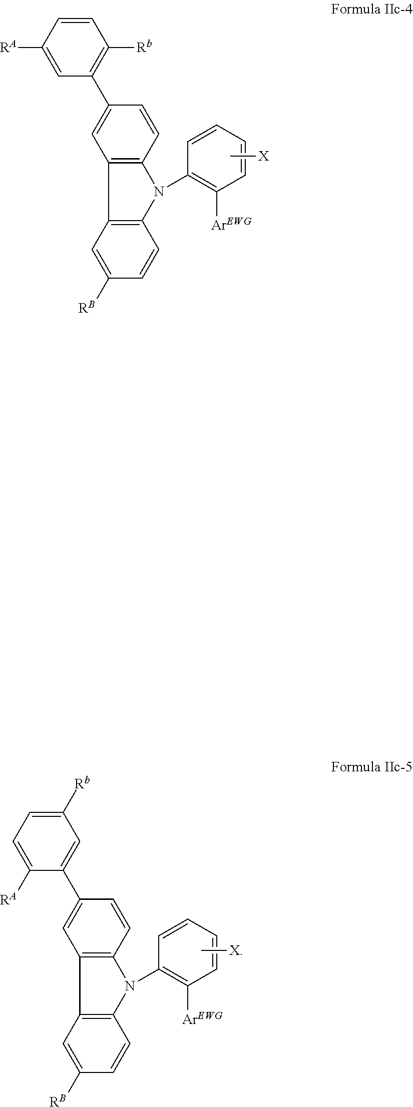

wherein the aforementioned definitions apply.

In a further embodiment of the invention, the organic molecules according to the invention comprise or consist of a structure which is selected from the group consisting of Formula IIc-1, Formula IIc-2, and Formula IIc-3:

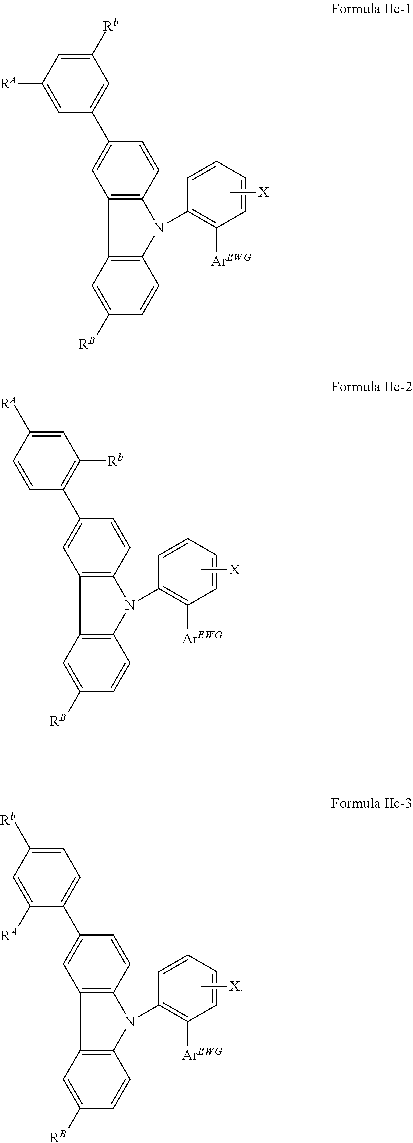

In another embodiment of the invention, the organic molecules according to the invention comprise or consist of a structure which is selected from the group consisting of Formula IIc-1, Formula IIc-2, Formula IIc-3, Formula IIc-4, and Formula IIc-5, wherein

R^{b} is selected independently of one another at each occurrence from the group consisting of H and CN.

In another embodiment of the invention, the organic molecules according to the invention comprise or consist of a structure which is selected from the group consisting of Formula IIc-1, Formula IIc-2, and Formula IIc-3, wherein

R^{b} is selected at each occurrence from the group consisting of H and CN.

In another embodiment of the invention, the organic molecules according to the invention comprise or consist of a structure which is selected from the group consisting of Formula IIc-1, Formula IIc-2, Formula IIc-3, Formula IIc-4, and Formula IIc-5, wherein

R^{b} is H.

In another embodiment of the invention, the organic molecules according to the invention comprise or consist of a structure which is selected from the group consisting of Formula IIc-1, Formula IIc-2, and Formula IIc-3, wherein

R^{b} is H.

In another embodiment of the invention, the organic molecules according to the invention comprise or consist of a structure which is selected from the group consisting of Formula IIc-1, Formula IIc-2, Formula IIc-3, Formula IIc-4, and Formula IIc-5, wherein

R^{b} is CN.

In another embodiment of the invention, the organic molecules according to the invention comprise or consist of a structure which is selected from the group consisting of Formula IIc-1, Formula IIc-2, and Formula IIc-3, wherein

R^{b} is CN.

In a preferred embodiment of the invention, the organic molecules according to the invention comprise or consist of a structure according to Formula IIc-1:

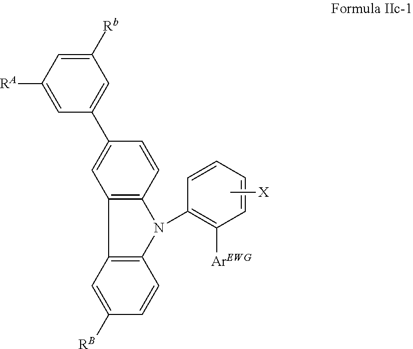

wherein R^{b} is selected at each occurrence from the group consisting of

H and CN.

In a preferred embodiment of the invention, the organic molecules according to the invention comprise or consist of the group shown below:

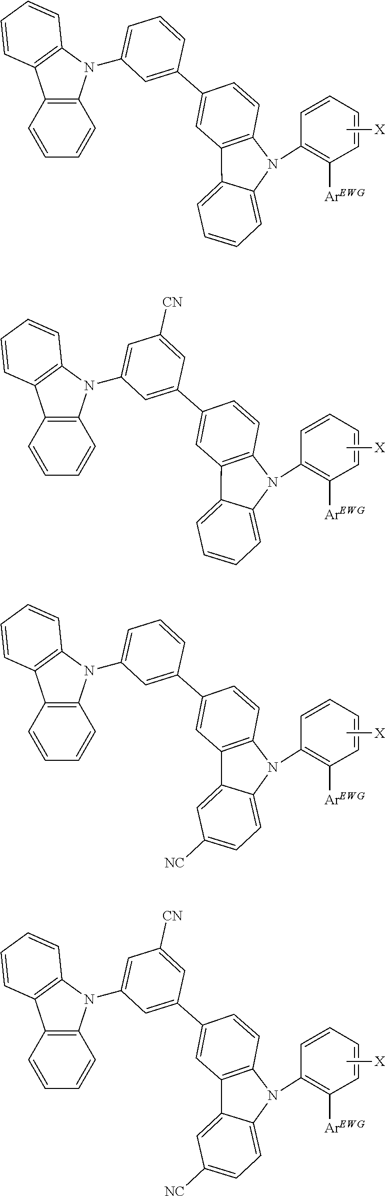

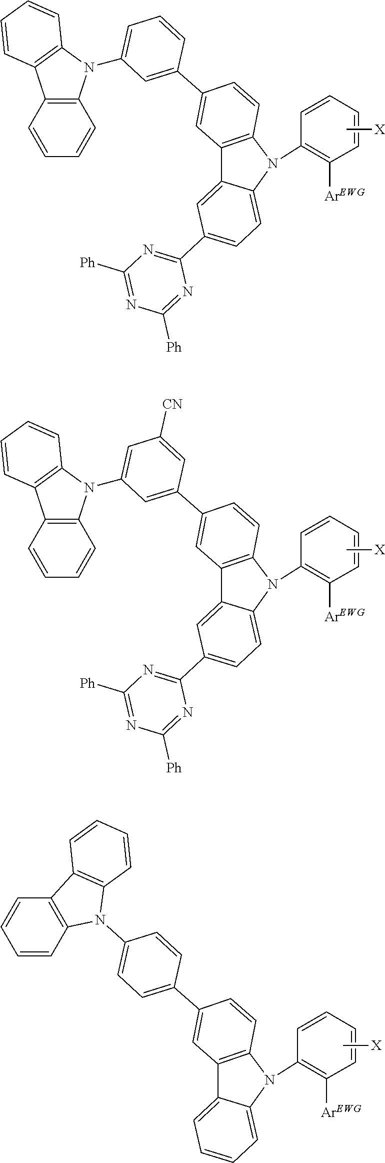

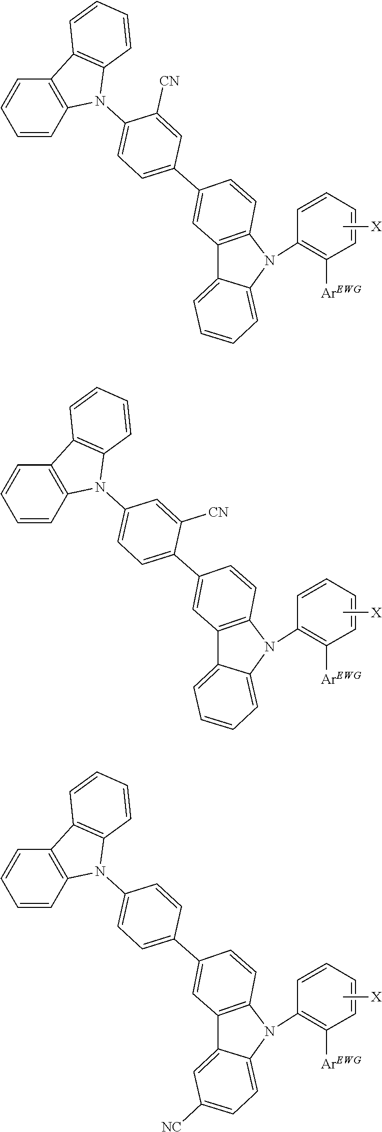

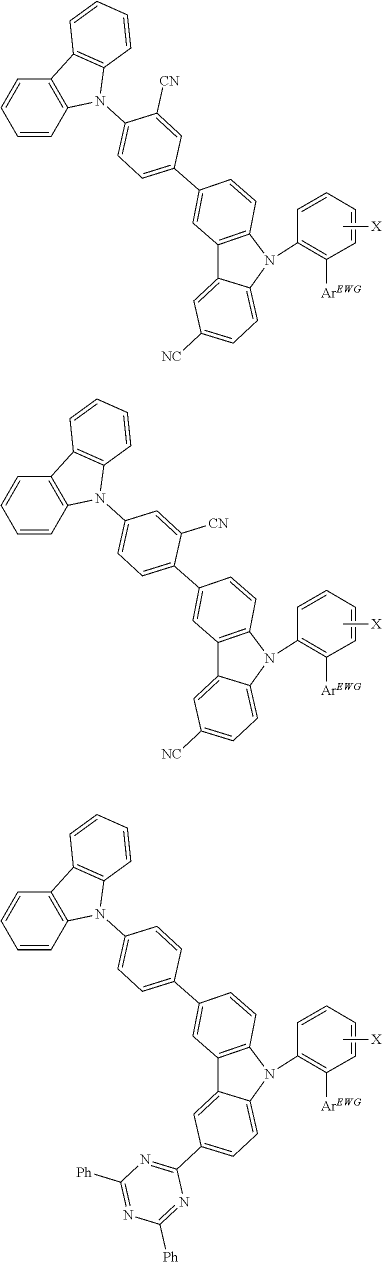

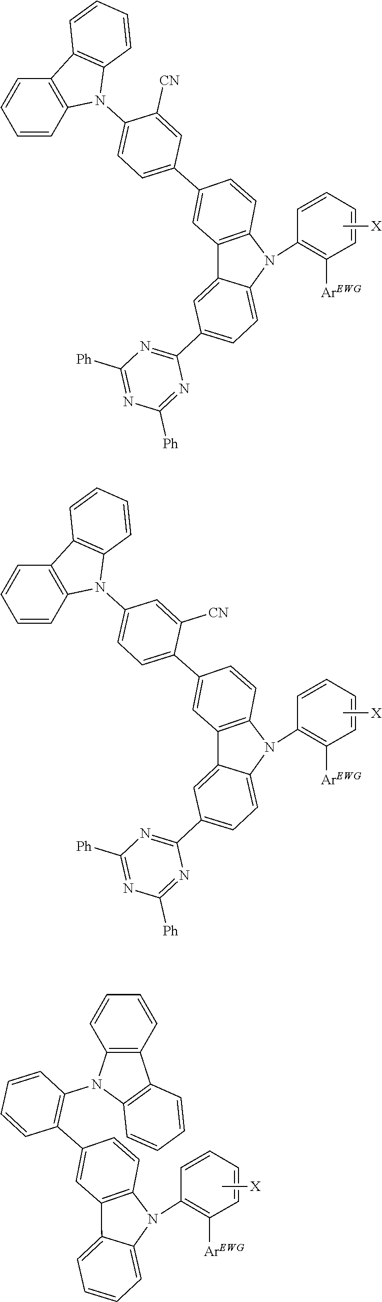

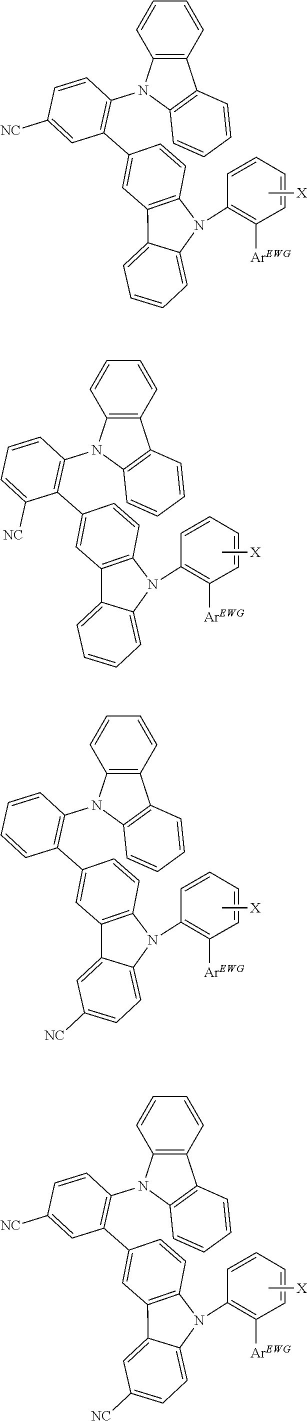

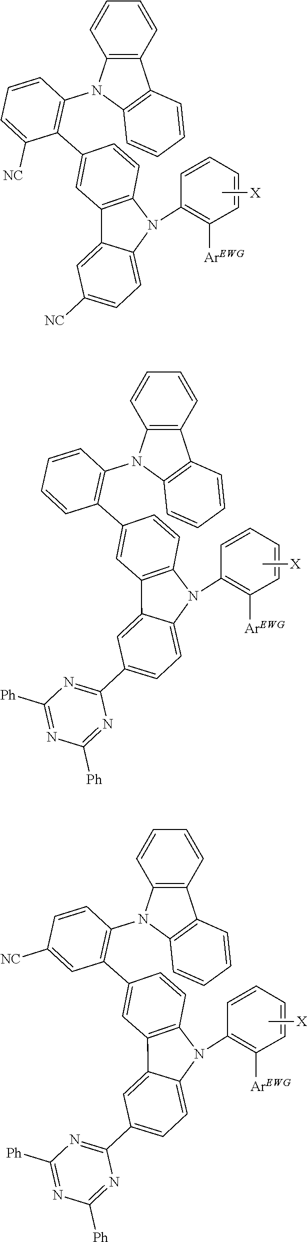

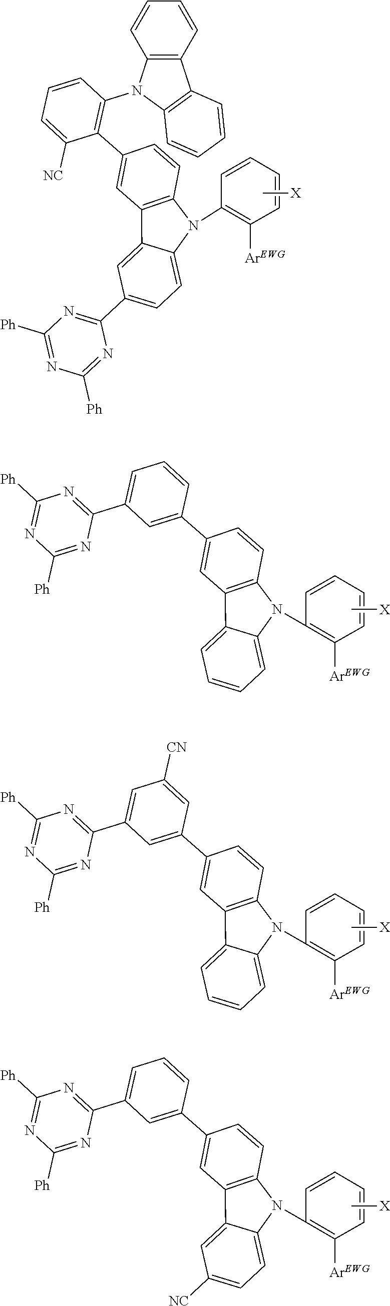

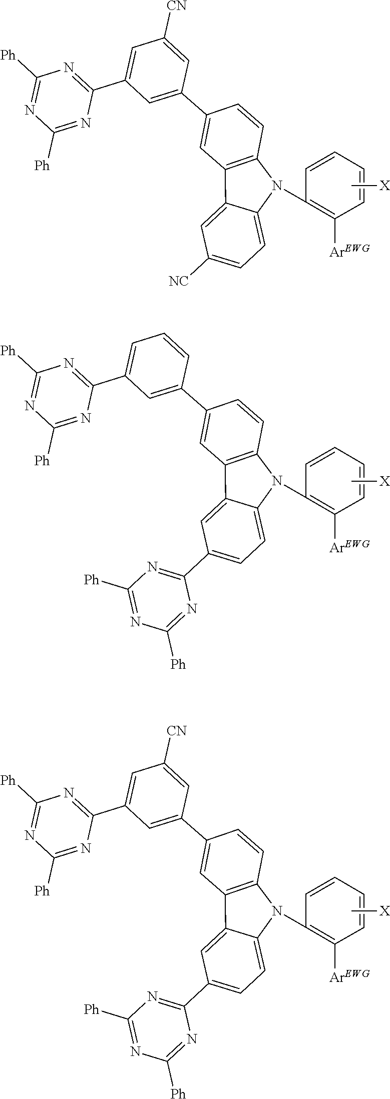

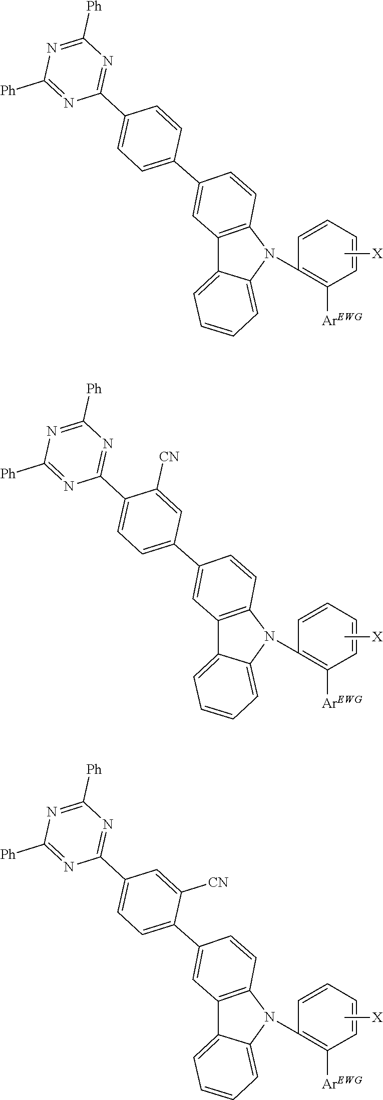

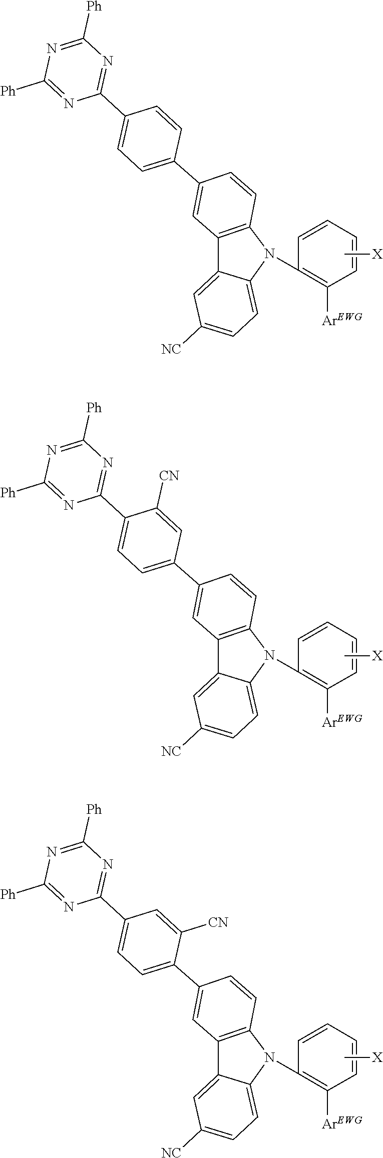

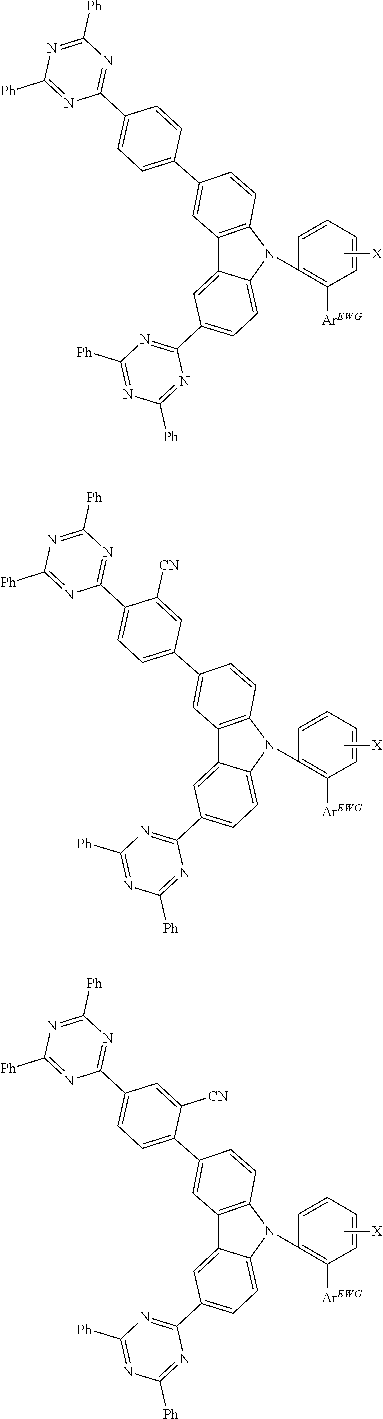

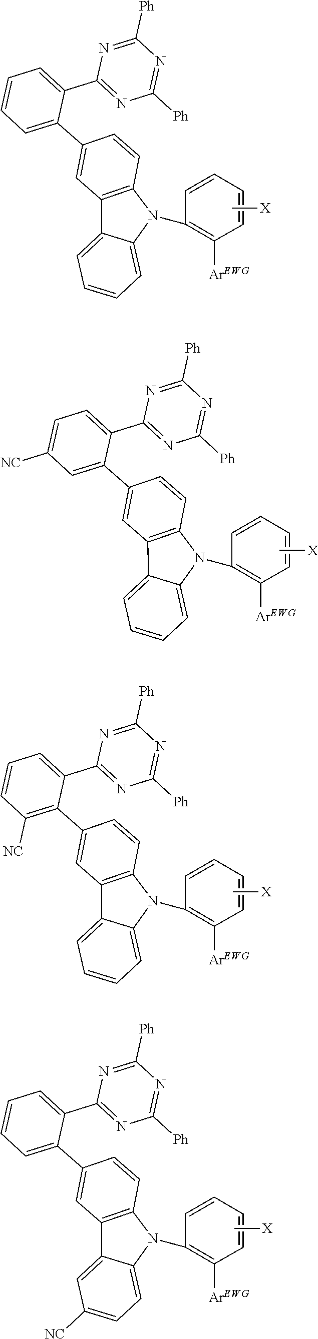

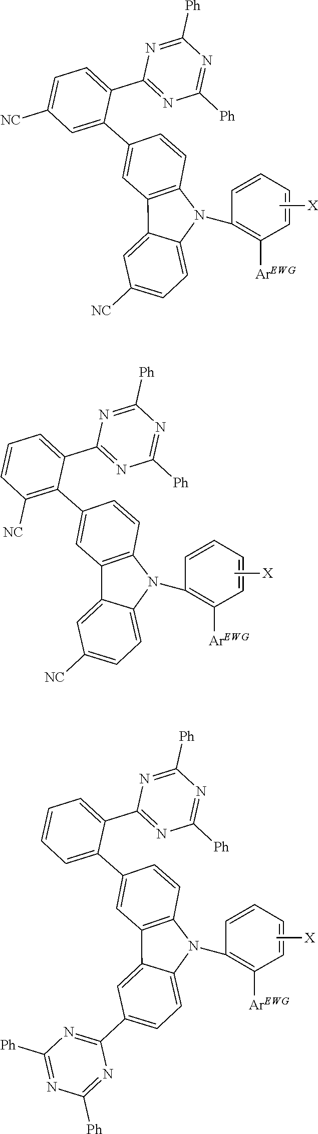

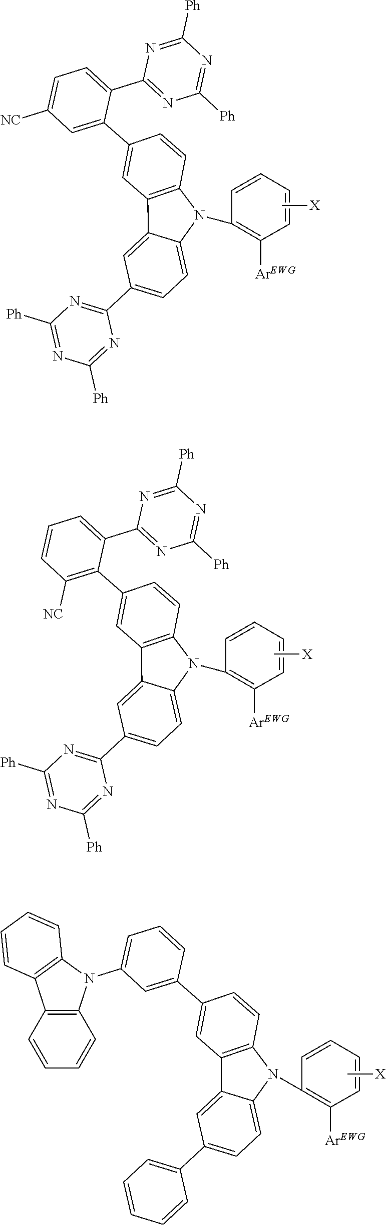

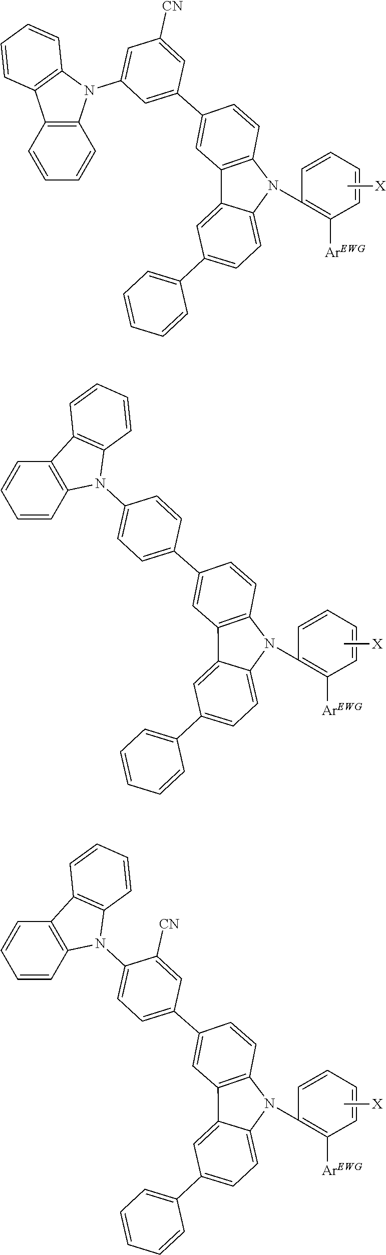

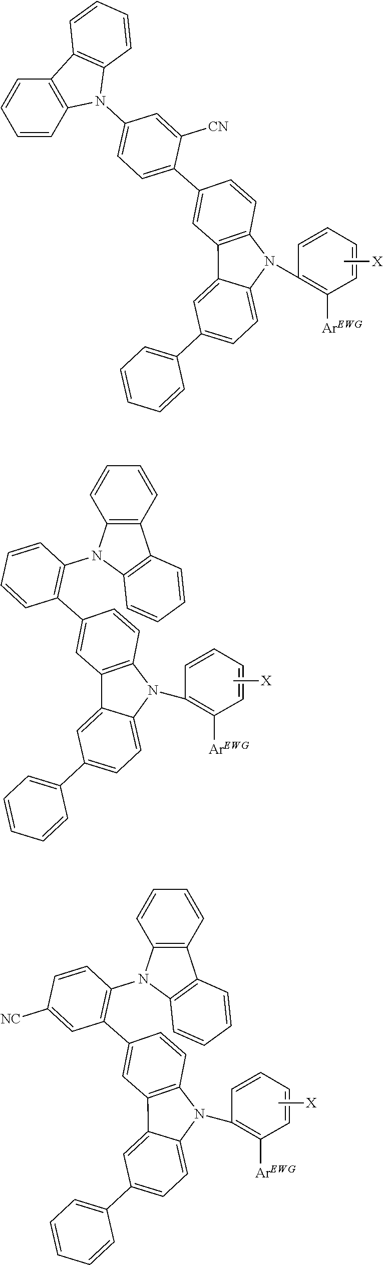

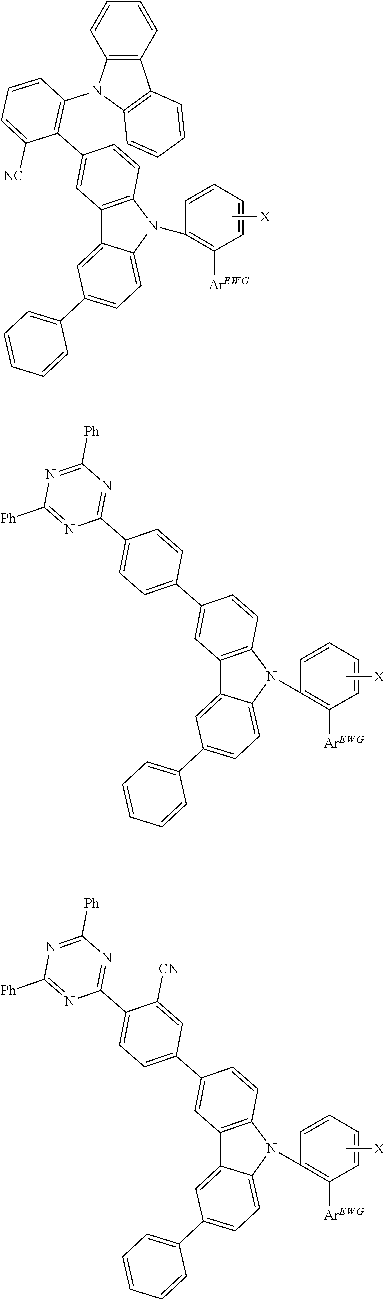

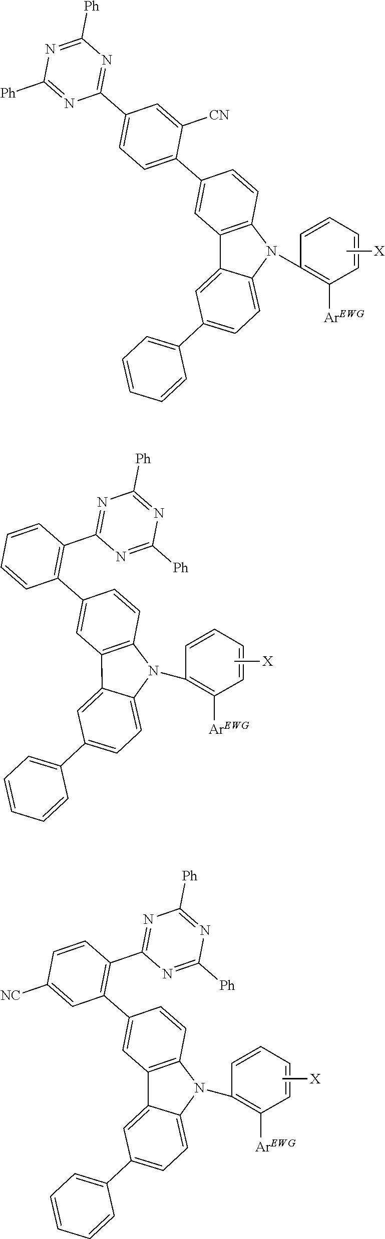

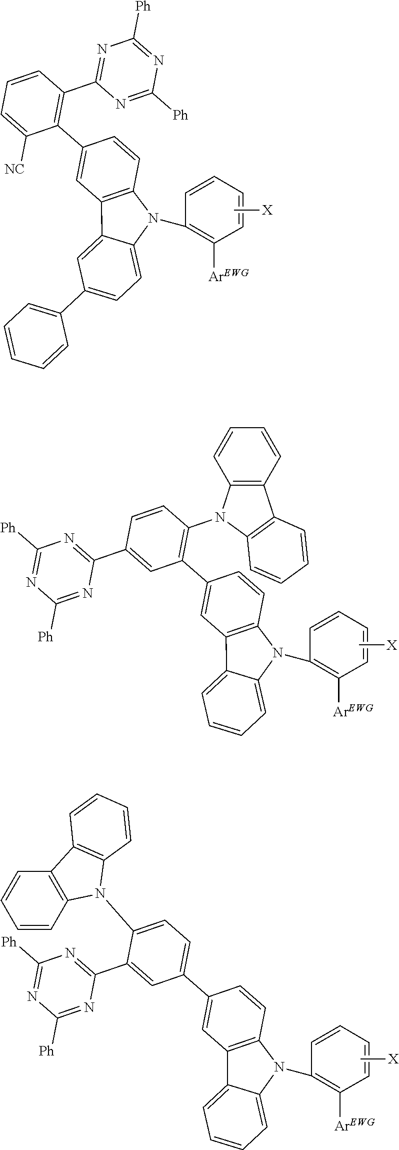

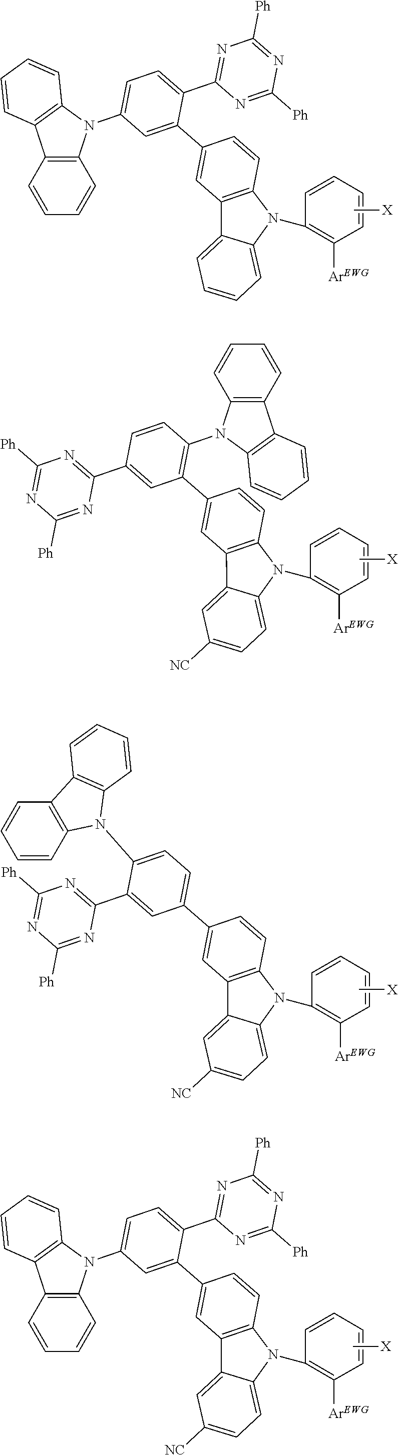

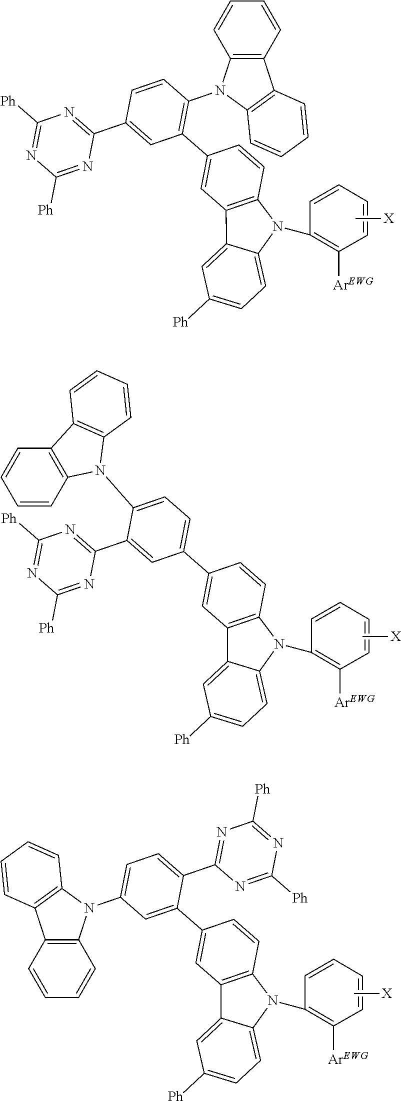

In a preferred embodiment of the invention, the organic molecules comprise a structure according to Formula IB or Formula IC or consist thereof:

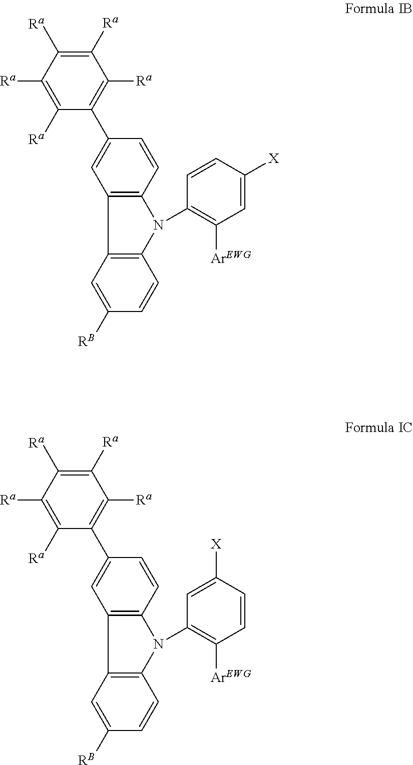

In a preferred embodiment of the invention, the organic molecules comprise a structure according to Formula IB.

In one embodiment of the invention, the organic molecules comprise a structure selected from the group consisting of Formula IVa-Formula IVe or consist thereof:

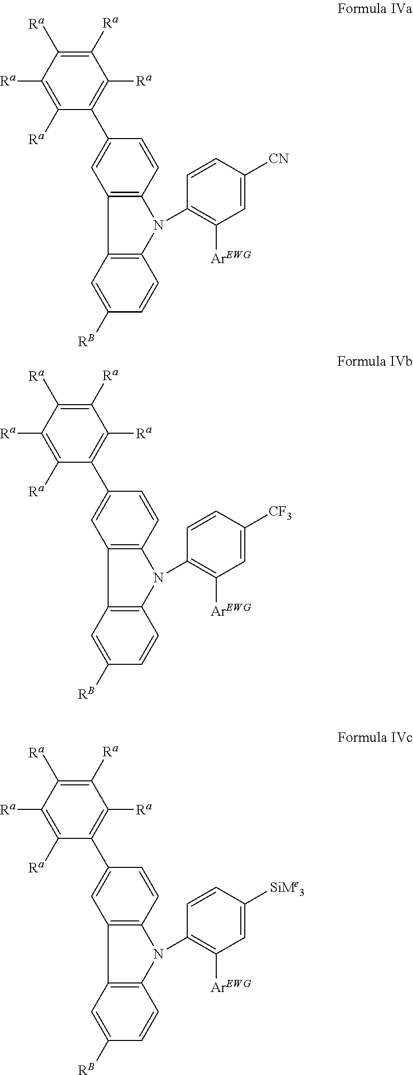

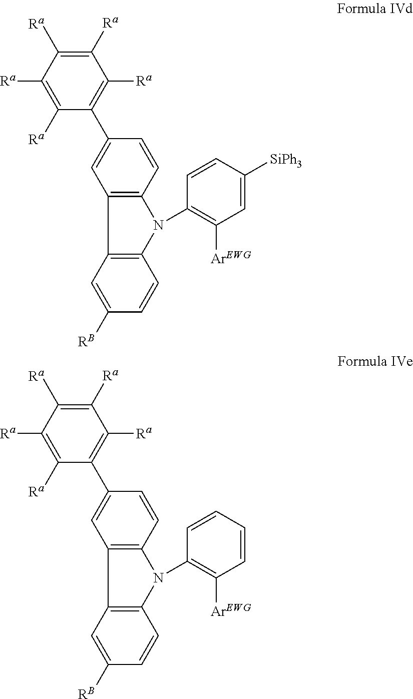

wherein the aforementioned definitions apply.

In a preferred embodiment of the invention, Ar^{EWG} is selected from the group consisting of

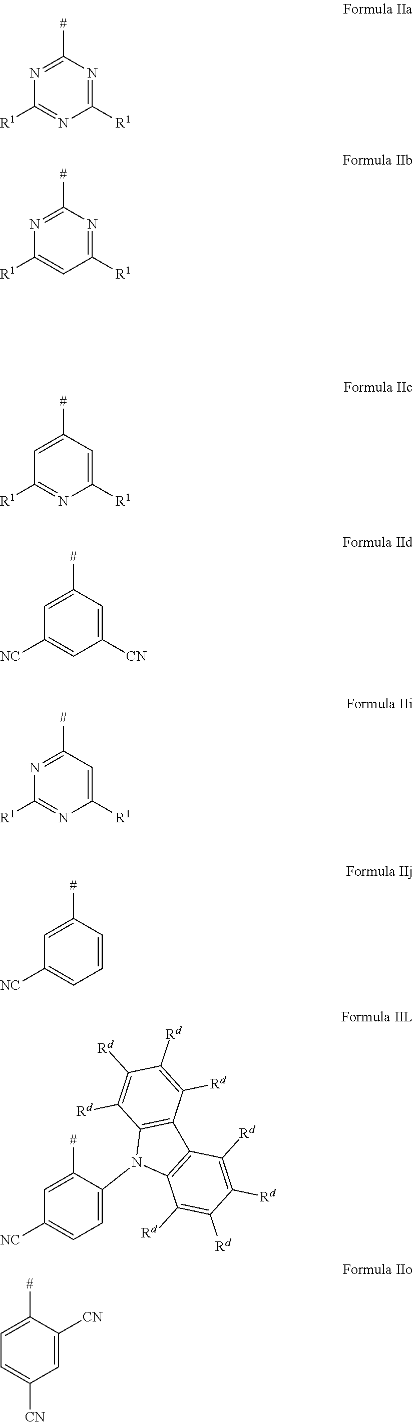

In a preferred embodiment of the invention, Ar^{EWG} is selected from the group consisting of

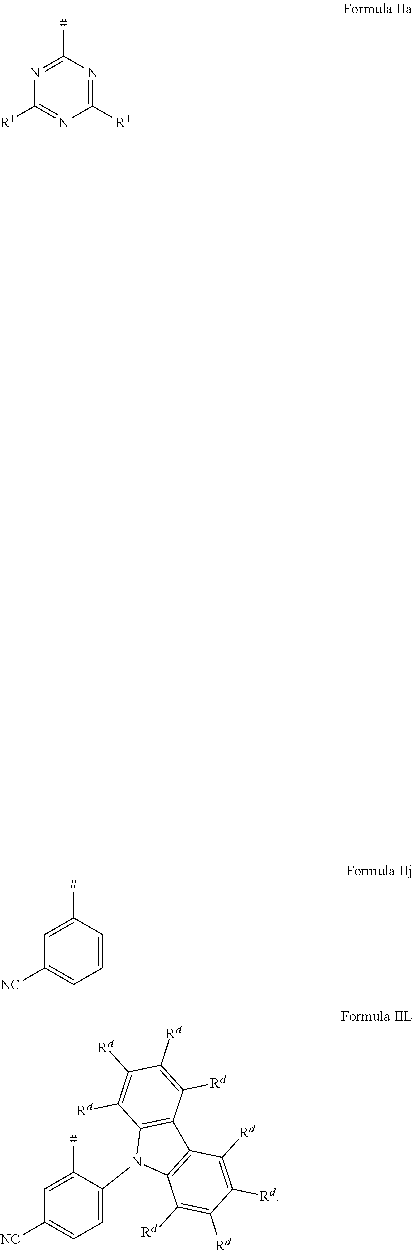

In one embodiment of the invention, the organic molecules comprise a structure selected from the group consisting of Formula IVa-1 to Formula IVa-15 or consist thereof:

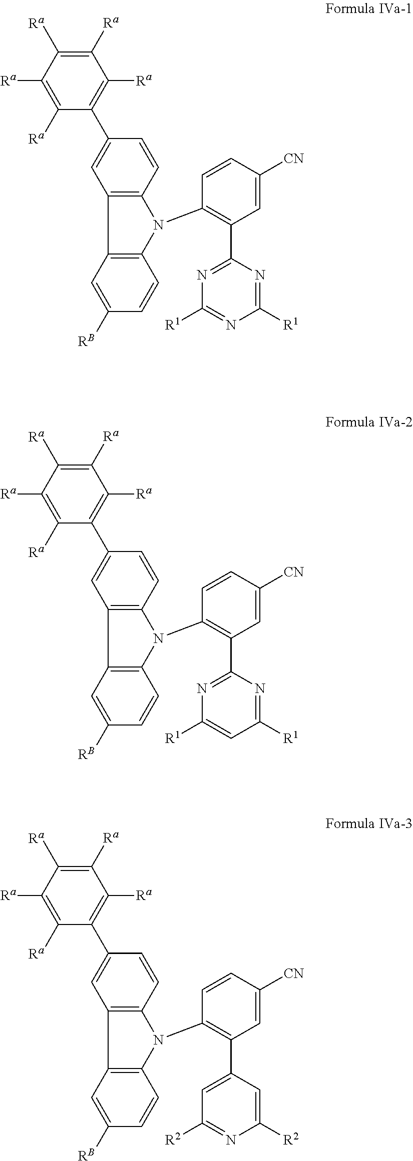

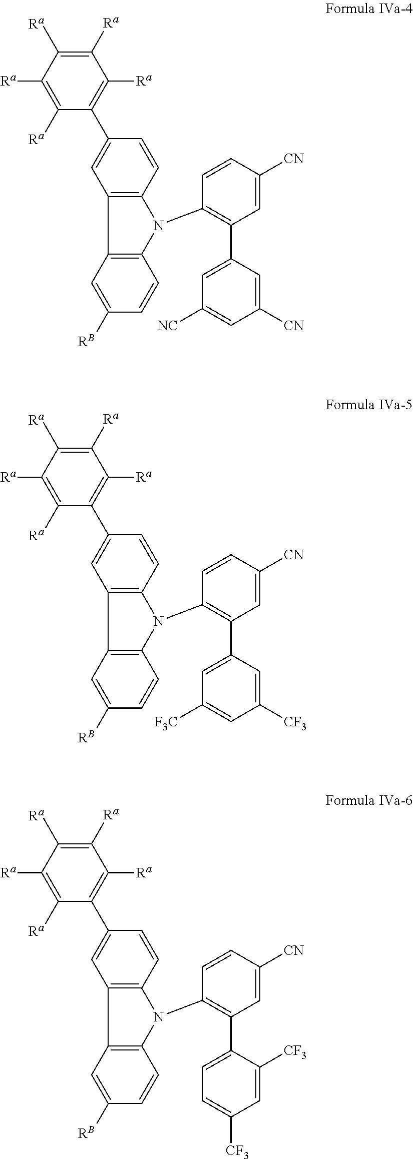

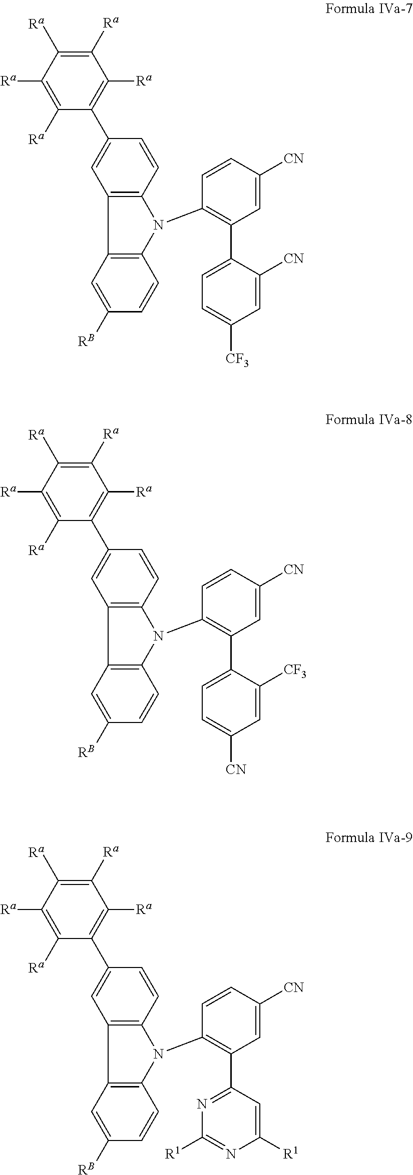

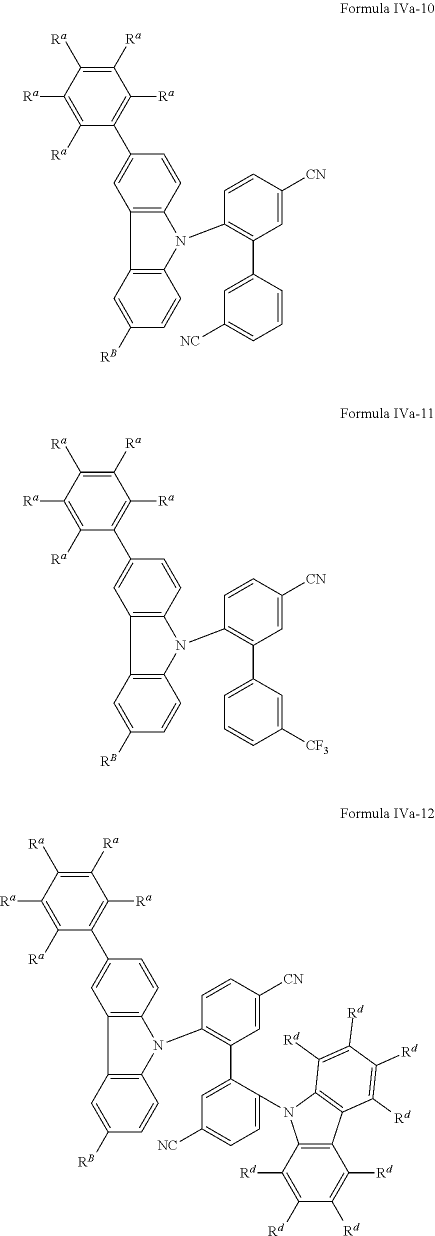

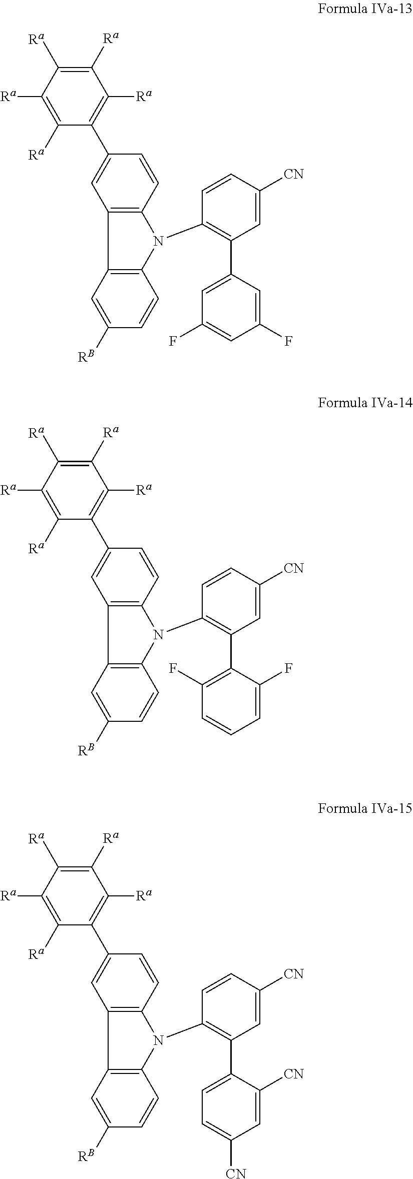

wherein the aforementioned definitions apply.

In one embodiment of the invention, the organic molecules comprise a structure selected from the group consisting of Formula IVb-1 to Formula IVb-15 or consist thereof:

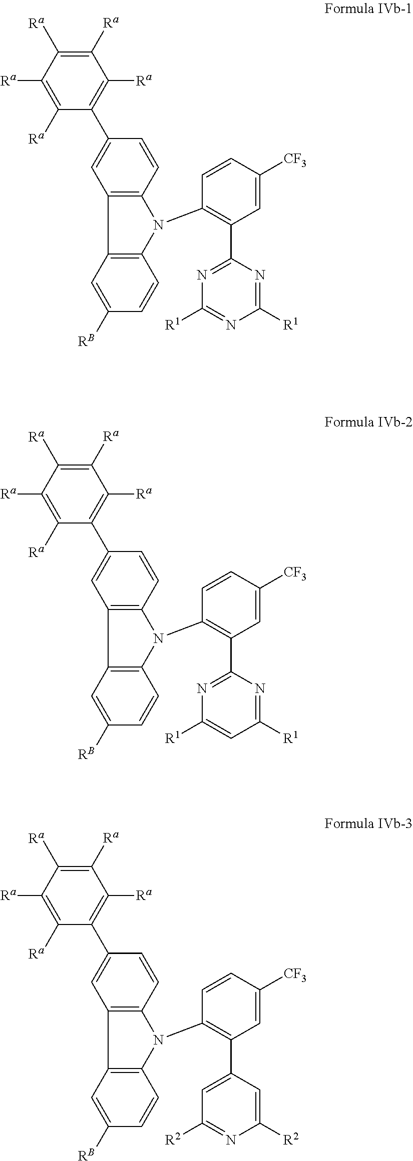

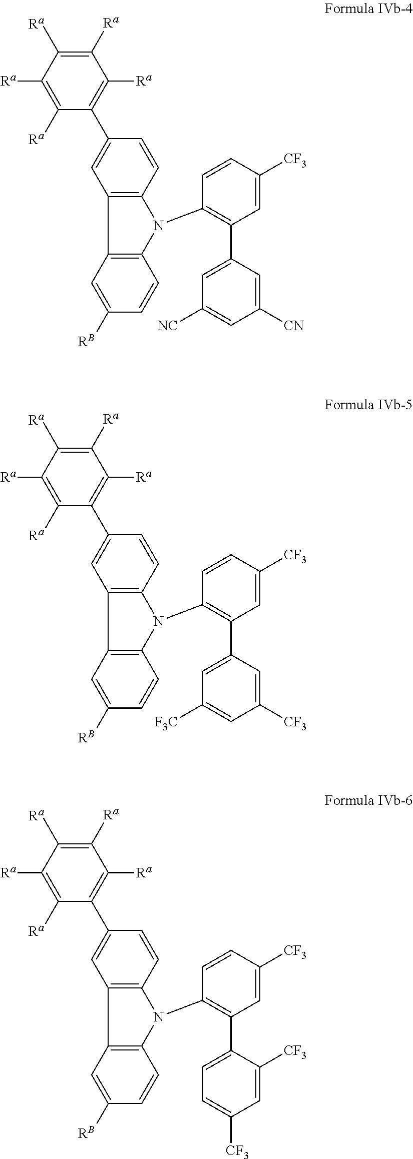

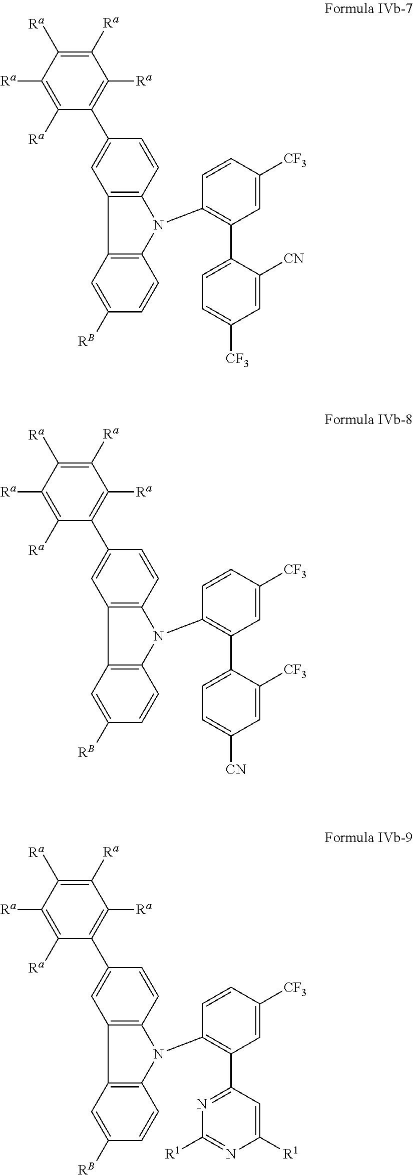

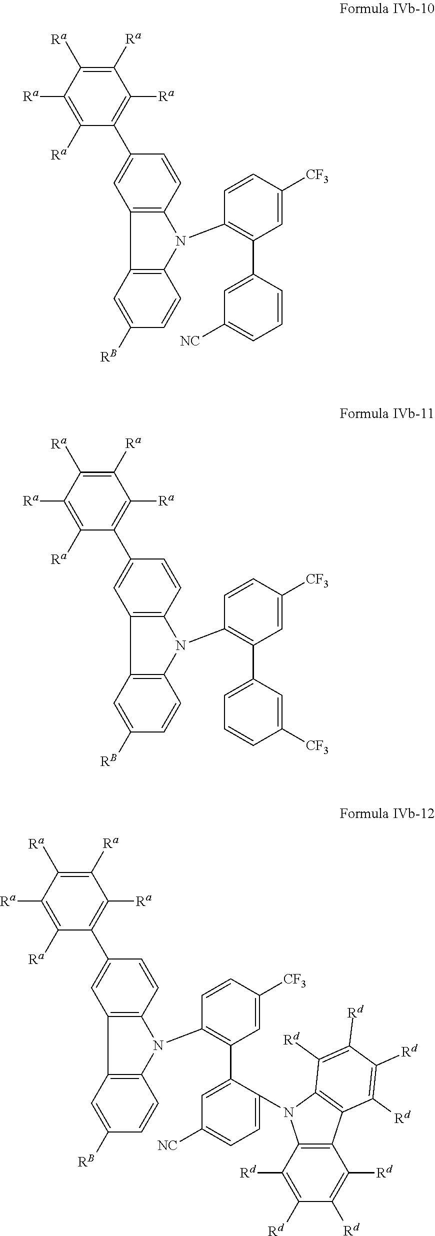

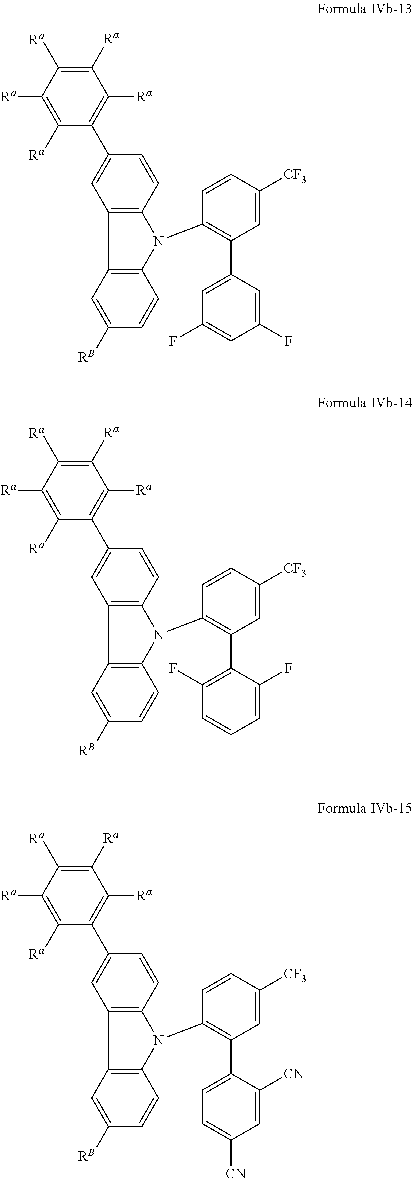

wherein the aforementioned definitions apply.

In one embodiment of the invention, the organic molecules comprise a structure selected from the group consisting of Formula IVc-1 to Formula IVc-12 or consist thereof:

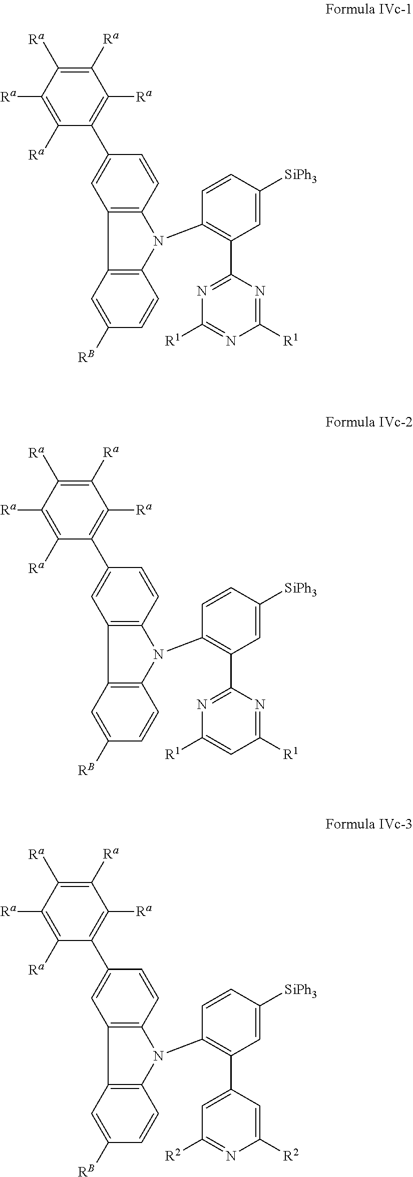

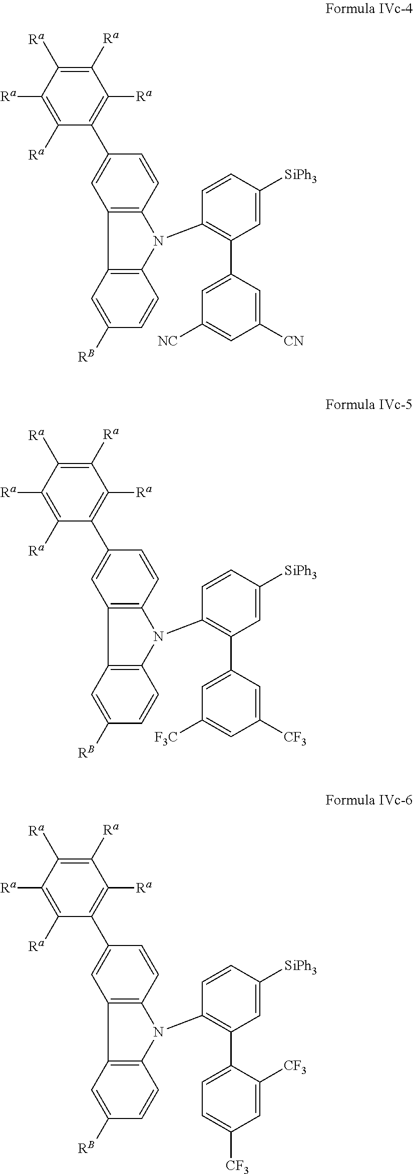

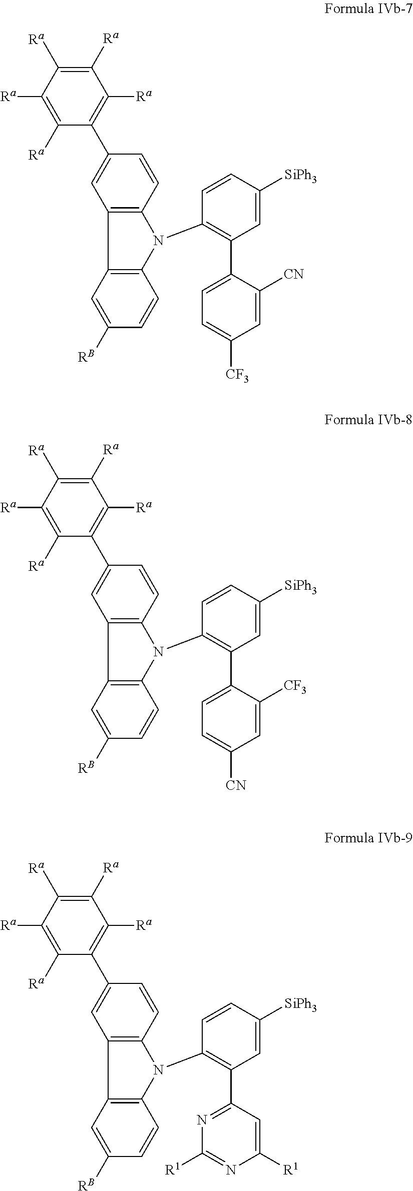

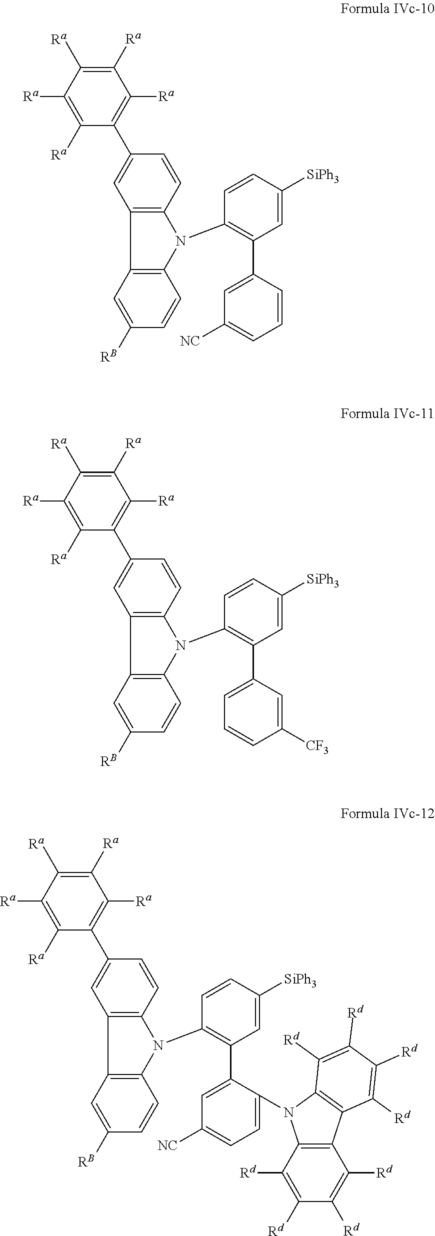

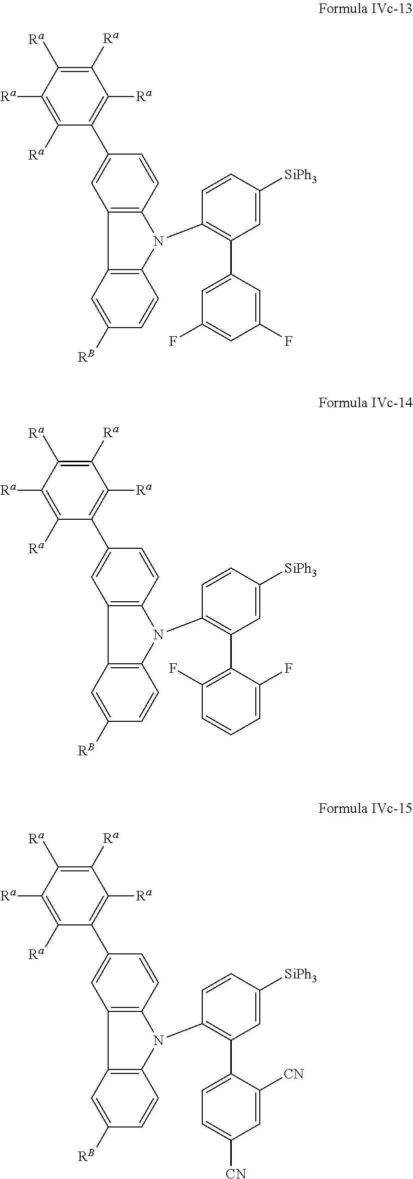

wherein the aforementioned definitions apply.

In one embodiment of the invention, the organic molecules comprise a structure selected from the group consisting of Formula IVd-1 to Formula IVd-12 or consist thereof:

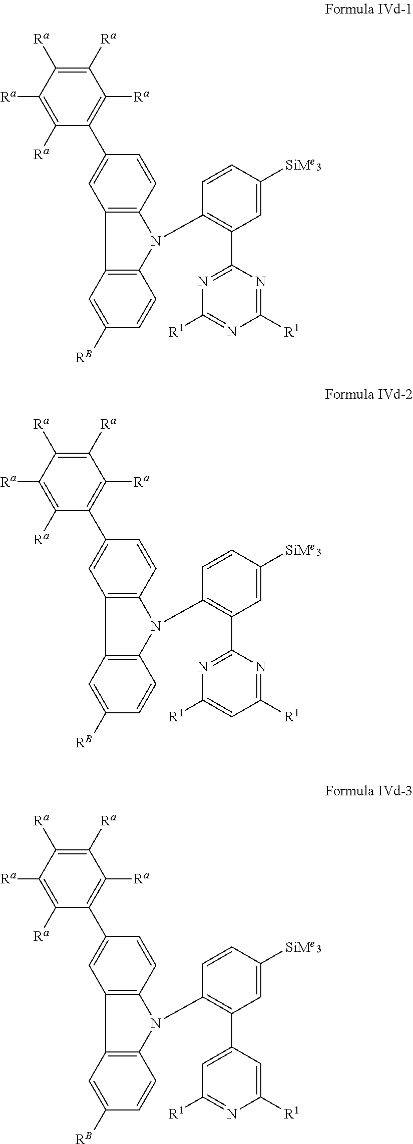

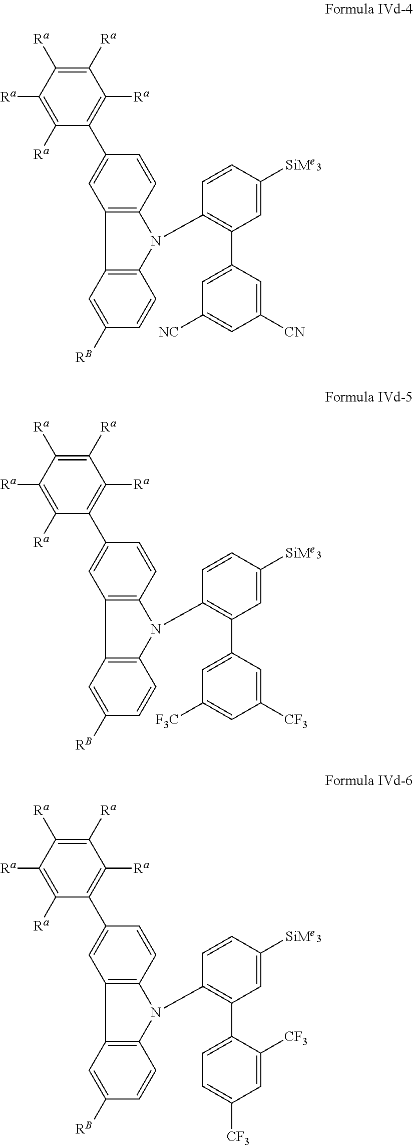

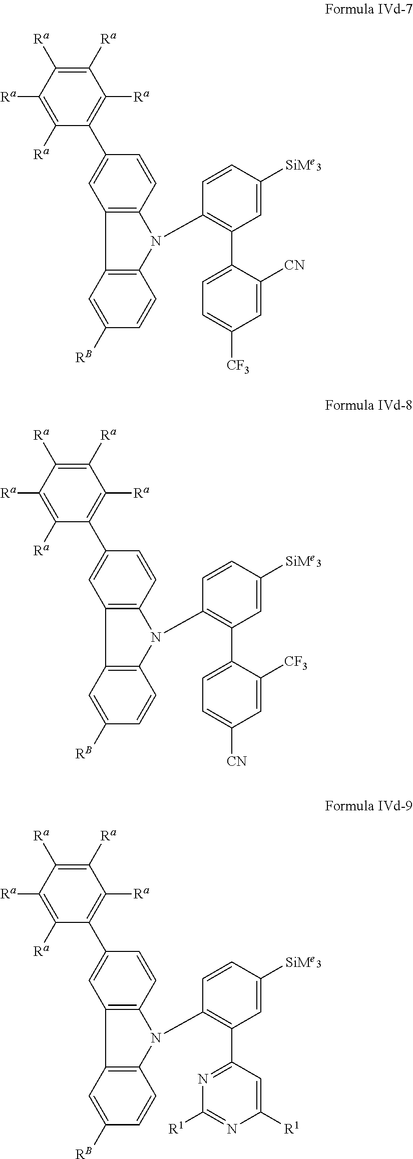

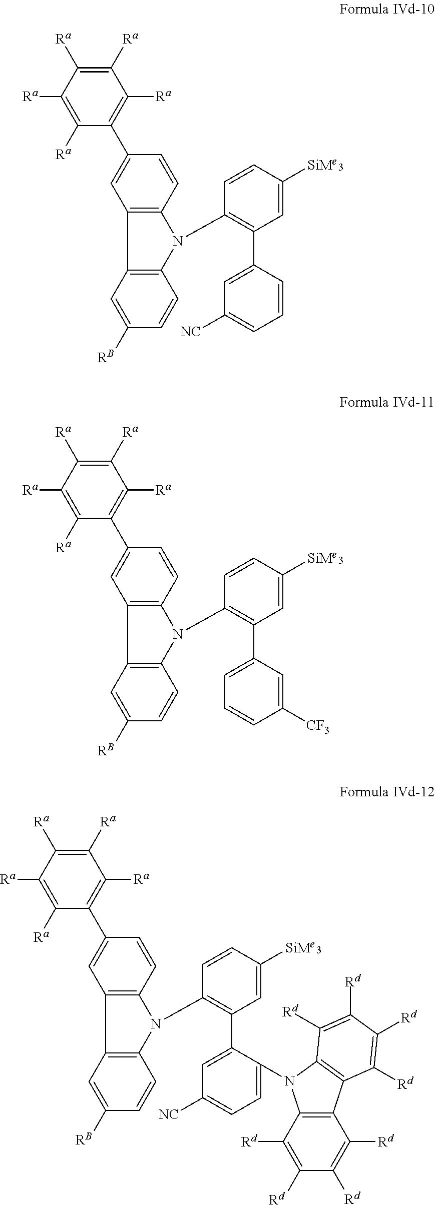

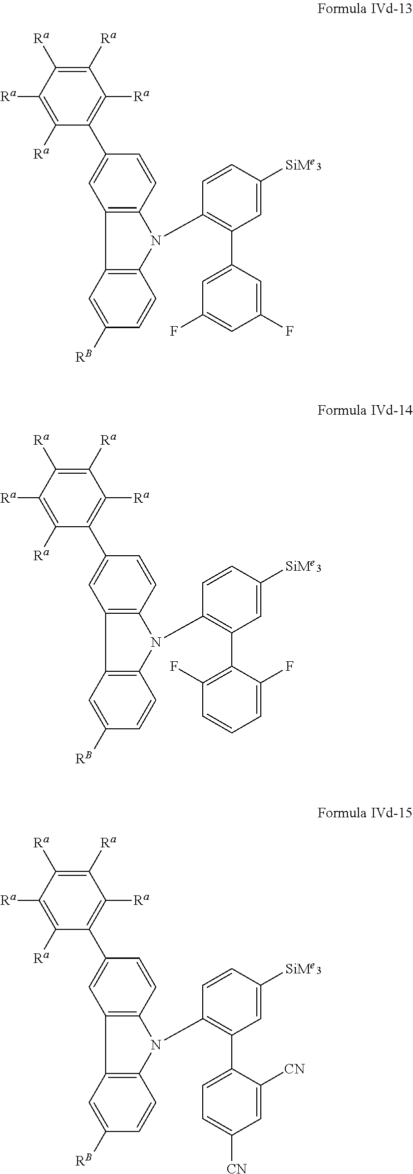

wherein the aforementioned definitions apply.

In one embodiment of the invention, the organic molecules comprise a structure selected from the group consisting of Formula IVe-1 to Formula IVe-12 or consist thereof:

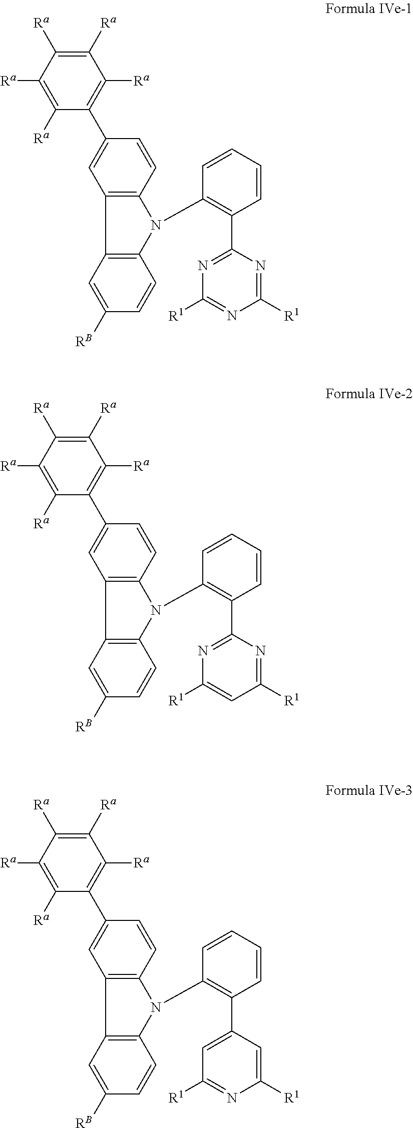

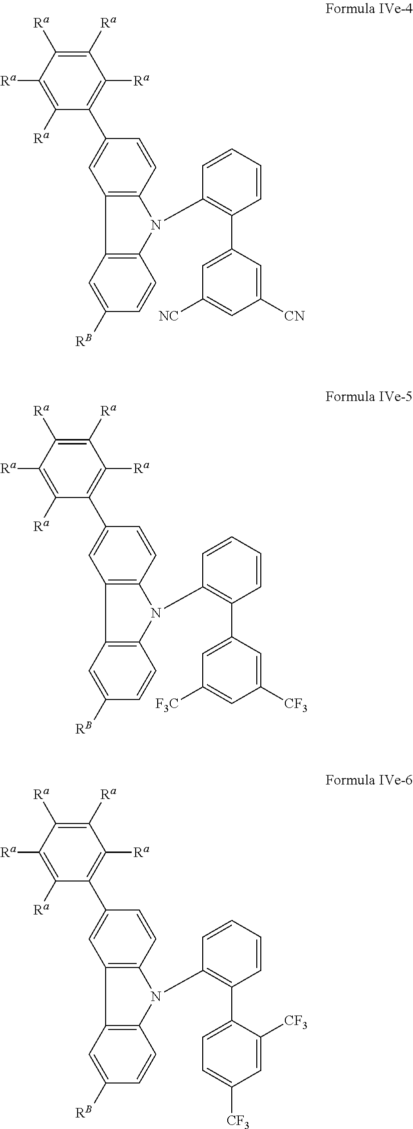

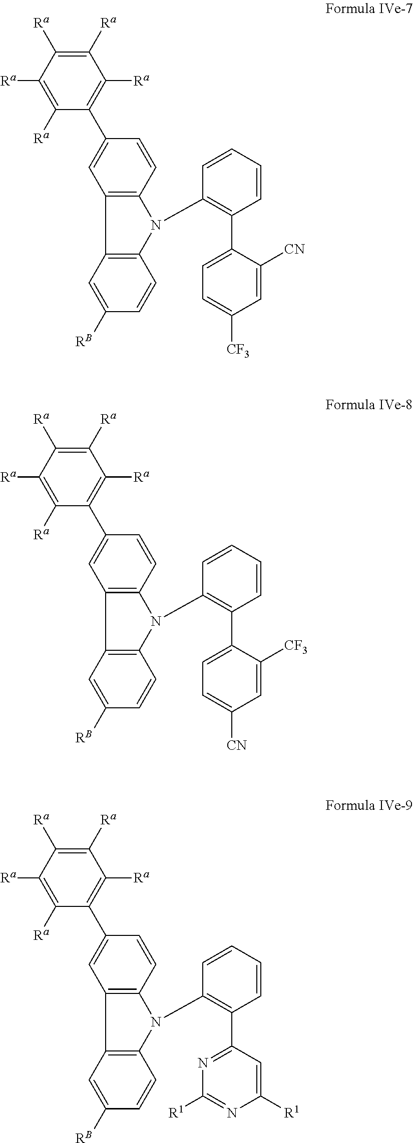

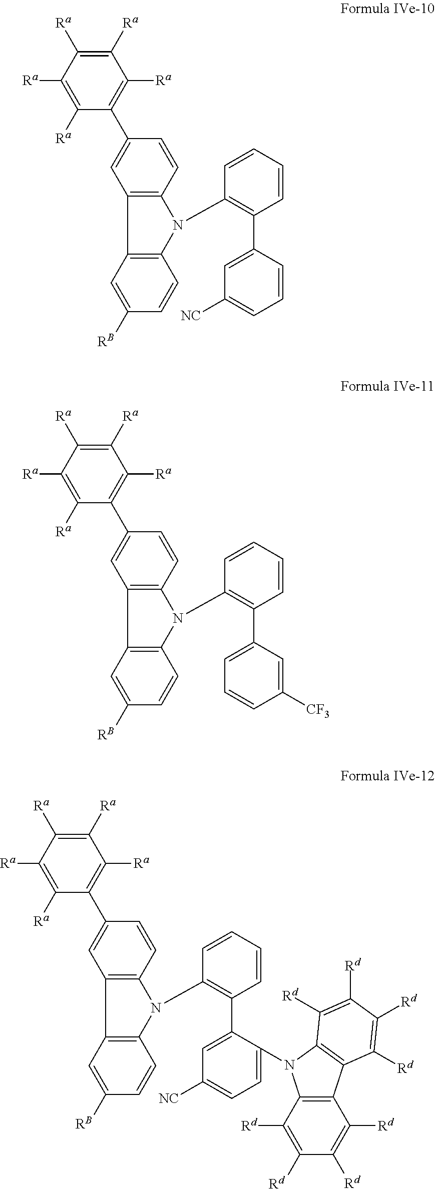

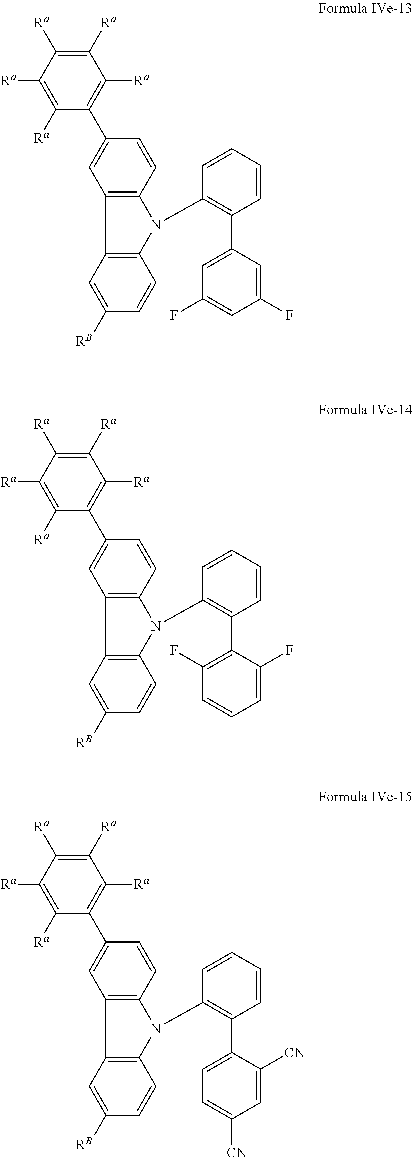

wherein the aforementioned definitions apply.

In one embodiment, the organic molecules according to the invention comprise or consist of a structure of Formula VII:

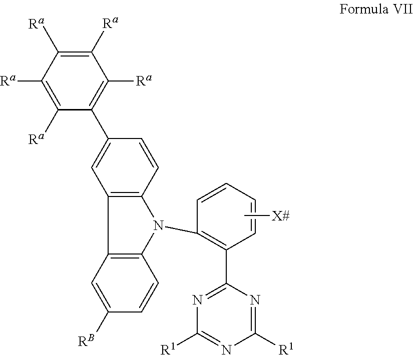

wherein X^{#} is selected from the group consisting of H, CN, and CF₃.

In one embodiment, the organic molecules according to the invention comprise or consist of a structure of Formula VII.

In a preferred embodiment, the organic molecules according to the invention comprise or consist of a structure of Formula VII, wherein X^{#} is CN.

The terms “aryl” and “aromatic” as used throughout the description can be understood in the broadest sense to be any mono-, bi-, or polycyclic aromatic component. An aryl group accordingly contains 6 to 60 aromatic ring atoms, and a heteroaryl group contains 5 to 60 aromatic ring atoms, at least one of which is a heteroatom. The number of aromatic ring atoms can nonetheless be given as a subscript number in the definition of specific substituents throughout the description. The heteroaromatic ring in particular contains one to three heteroatoms. The terms “heteroaryl” and “heteroaromatic” can be understood in the broadest sense to be any mono-, bi-, or polycyclic heteroaromatic component that contains at least one heteroatom. The heteroatoms can be the same or different at each occurrence and can be individually selected from the group consisting of N, O, and S. The term “arylene” accordingly refers to a divalent substituent which bears two binding sites to other molecular structures and thus serves as a linker structure. If a group is defined differently in the exemplary embodiments than the definitions given here, for example if the number of aromatic ring atoms or the number of heteroatoms differs from the given definition, the definition in the exemplary embodiments must be applied. According to the invention, a condensed (annulated) aromatic or heteroaromatic polycyclic compound consists of two or more individual aromatic or heteroaromatic cycles that have formed the polycyclic compound via a condensation reaction.

As used throughout the description in particular, the term “aryl group” or “heteroaryl group” includes groups which can be bound via any position of the aromatic or heteroaromatic group, derived from benzene, naphthalene, anthracene, phenanthrene, pyrene, dihydropyrene, chrysene, perylene, fluoranthene, benzanthracene, benzophenanthrene, tetracene, pentacene, benzopyrene, furan, benzofuran, isobenzofuran, dibenzofuran, thiophene, benzothiophene, isobenzothiophene, dibenzothiophene; pyrrole, indole, isoindole, carbazole, pyridine, quinoline, isoquinoline, acridine, phenanthridine, benzo-5,6-quinoline, benzo-6,7-quinoline, benzo-7,8-quinoline, phenothiazine, phenoxazine, pyrazole, indazole, imidazole, benzimidazole, naphthoimidazole, phenanthroimidazole, pyridoimidazole, pyrazinoimidazole, quinoxalinoimidazole, oxazole, benzoxazole, napthooxazole, anthroxazole, phenanthroxazole, isoxazole, 1,2-thiazole, 1,3-thiazole, benzothiazole, pyridazine, benzopyridazine, pyrimidine, benzopyrimidine, 1,3,5-triazine, quinoxaline, pyrazine, phenazine, naphthyridine, carboline, benzocarboline, phenanthroline, 1,2,3-triazole, 1,2,4-triazole, benzotriazole, 1,2,3-oxadiazole, 1,2,4-oxadiazole, 1,2,5-oxadiazole, 1,2,3,4-tetrazine, purine, pteridine, indolizine, and benzothiadiazole, or combinations of the aforementioned groups.

As used throughout the present description, the term “cyclic group” can be understood in the broadest sense to be mono-, bi-, or polycyclic components.

As used throughout the present description, the term “alkyl group” can be understood in the broadest sense to be any linear, branched, or cyclic alkyl substituent. The term “alkyl” in particular includes the substituents methyl (Me), ethyl (Et), n-propyl (ⁿPr), i-propyl (ⁱPr), cyclopropyl, n-butyl (ⁿBu), i-butyl (ⁱBu), s-butyl (^{s}Bu), t-butyl (^{t}Bu), cyclobutyl, 2-methylbutyl, n-pentyl, s-pentyl, t-pentyl, 2-pentyl, neo-pentyl, cyclopentyl, n-hexyl, s-hexyl, t-hexyl, 2-hexyl, 3-hexyl, neo-hexyl, cyclohexyl, 1-methylcyclopentyl, 2-methylpentyl, n-heptyl, 2-heptyl, 3-heptyl, 4-heptyl, cycloheptyl, 1-methylcyclohexyl, n-octyl, 2-ethylhexyl, cyclooctyl, 1-bicyclo[2,2,2]octyl, 2-bicyclo[2,2,2]-octyl, 2-(2,6-dimethyl)octyl, 3-(3,7-dimethyl)octyl, adamantyl, 2,2,2-trifluoroethyl, 1,1-dimethyl-n-hex-1-yl, 1,1-dimethyl-n-hept-1-yl, 1,1-dimethyl-n-oct-1-yl, 1,1-dimethyl-n-dec-1-yl, 1,1-dimethyl-n-dodec-1-yl, 1,1-dimethyl-n-tetradec-1-yl, 1,1-dimethyl-n-hexadec-1-yl, 1,1-dimethyl-n-octadec-1-yl, 1,1-diethyl-n-hex-1-yl, 1,1-diethyl-n-hept-1-yl, 1,1-diethyl-n-oct-1-yl, 1,1-diethyl-n-dec-1-yl, 1,1-diethyl-n-dodec-1-yl, 1,1-diethyl-n-tetradec-1-yl, 1,1-diethyl-n-hexadec-1-yl, 1,1-diethyl-n-octadec-1-yl, 1-(n-propyl)-cyclohex-1-yl, 1-(n-butyl)-cyclohex-1-yl, 1-(n-hexyl)-cyclohex-1-yl, 1-(n-octyl)-cyclohex-1-yl, and 1-(n-decyl)-cyclohex-1-yl.

As used throughout the present description, the term “alkenyl” comprises linear, branched, and cyclic alkenyl substituents. For example, the term “alkenyl group” includes the substituents ethenyl, propenyl, butenyl, pentenyl, cyclopentenyl, hexenyl, cyclohexenyl, heptenyl, cycloheptenyl, octenyl, cyclooctenyl, or cyclooctadienyl.

As used throughout the description, the term “alkynyl” includes linear, branched, and cyclic alkynyl substituents. The term “alkynyl group” includes, for example, ethinyl, propinyl, butinyl, pentinyl, hexinyl, heptinyl, or octinyl.

As used throughout the description, the term “alkoxy” includes linear, branched, and cyclic alkoxy substituents. For example, the term “alkoxy group” includes methoxy, ethoxy, n-propoxy, i-propoxy, n-butoxy, i-butoxy, s-butoxy, t-butoxy, and 2-methylbutoxy.

As used throughout the description, the term “thioalkoxy” includes linear, branched, and cyclic thioalkoxy substituents in which the 0 of the exemplary alkoxy groups is replaced by S.

As used throughout the description, the terms “halogen” and “halo” can be understood in the broadest sense to preferably be fluorine, chlorine, bromine, or iodine.

Whenever hydrogen is mentioned herein, it can at each occurrence also be replaced by deuterium.

It goes without saying that when a molecular fragment is described as being a substituent or otherwise attached to another component, its name can be written as if it were a fragment (e.g., naphthyl, dibenzofuryl) or as if it were the entire molecule (e.g., naphthalene, dibenzofuran). As used herein, these different ways of designating a substituent or attached fragment are considered to be equivalent.

In one embodiment, the organic molecules according to the invention have an excited state lifetime of no more than 150 μs, no more than 100 μs, in particular no more than 50 μs, more preferably no more than 10 μs or no more than 7 μs in a film of poly(methyl methacrylate) (PMMA) with 10 wt % of the organic molecule at room temperature.

In one embodiment of the invention, the organic molecules according to the invention represent thermally activated delayed fluorescence (TADF) emitters that exhibit a ΔE_{ST} value, which corresponds to the energy difference between the first excited singlet state (S1) and the first excited triplet state (T1), of less than 5000 cm⁻¹, preferably less than 3000 cm⁻¹, more preferably less than 1500 cm⁻¹, even more preferably less than 1000 cm⁻¹, or even less than 500 cm⁻¹.

In another embodiment of the invention, the organic molecules according to the invention have an emission peak in the visible or near ultraviolet range, i.e., in the range of a wavelength of 380 to 800 nm, with a full width at half maximum of 0.50 eV, preferably less than 0.48 eV, more preferably less than 0.45 eV, even more preferably less than 0.43 eV, or even less than 0.40 eV in a film of poly(methyl methacrylate) (PMMA) with 10 wt % of the organic molecule at room temperature.

In another embodiment of the invention, the organic molecules according to the invention have a blue material index (BMI), calculated by dividing the photoluminescence quantum yield (PLQY) in by the CIEy color coordinate of the emitted light, of more than 150, in particular more than 200, preferably more than 250, more preferably more than 300, or even more than 500.

Orbital energies and excited state energies can be determined either by experimental methods or by calculations using quantum chemical methods, in particular calculations based on density functional theory. The energy of the highest occupied molecular orbital E^{HOMO} is determined by methods known to the person skilled in the art using cyclic voltammetry measurements with an accuracy of 0.1 eV. The energy of the lowest unoccupied molecular orbital E^{LUMO} is calculated as E^{HOMO}+E^{gap}, wherein E^{gap} is determined as follows: For host compounds, the offset of the emission spectrum of a film with 10 wt % of the host in poly(methyl methacrylate) (PMMA) is used as E^{gap} unless stated otherwise. For emitter molecules, E^{gap} is determined as the energy at which the excitation and emission spectra of a film with 10 wt % of the emitter in PMMA intersect.

The energy of the first excited triplet state T1 is determined using the offset of the emission spectrum at low temperature, typically at 77 K. For host compounds in which the first excited singlet state and the lowest triplet state are energetically separated by >0.4 eV, the phosphorescence is usually visible in a stationary spectrum in 2-Me-THF. The triplet energy can thus be determined as the offset of the phosphorescence spectrum. For TADF emitter molecules, the energy of the first excited triplet state T1 is determined based on the offset of the delayed emission spectrum at 77 K, unless otherwise measured in a PMMA film with 10 wt % of the emitter. For both host and emitter compounds, the energy of the first excited singlet state S1 is determined based on the offset of the emission spectrum, unless otherwise measured in a PMMA film with 10 wt % of the host or emitter compound. The offset of an emission spectrum is determined by calculating the intersection of the tangent to the emission spectrum with the x-axis. The tangent to the emission spectrum is established on the high energy side of the emission range, i.e., where the emission range increases by going from higher energy values to lower energy values, and at the point at half maximum of the maximum intensity of the emission spectrum.

A further aspect of the invention relates to a process for producing organic molecules according to the invention (with an optional subsequent reaction), wherein a palladium-catalyzed cross-coupling reaction is used:

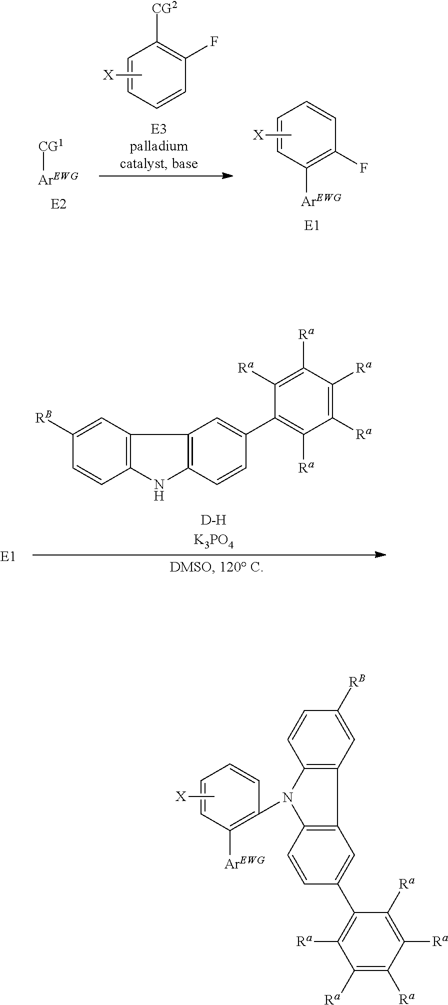

According to the invention, E2, an Ar^{EWG} group substituted with the coupling group CG¹, reacts with E3, a phenyl substituted with X, the coupling group CG², and once F. The coupling groups CG¹ and CG² are selected as a reaction pair such that the Ar^{EWG} group of E2 is introduced at the substitution position CG² of E3. A so-called Suzuki coupling is preferred here. In this case, either CG¹ is selected from Cl, Br, or I and CG² is selected from a boronic acid or a boronic acid ester, in particular a boronic acid pinacol ester, or CG² is analogously selected from Cl, Br, or I and CG¹ is selected from a boronic acid or a boronic acid ester, in particular a boronic acid pinacol ester.

D-H can be produced according to the following synthesis pathways

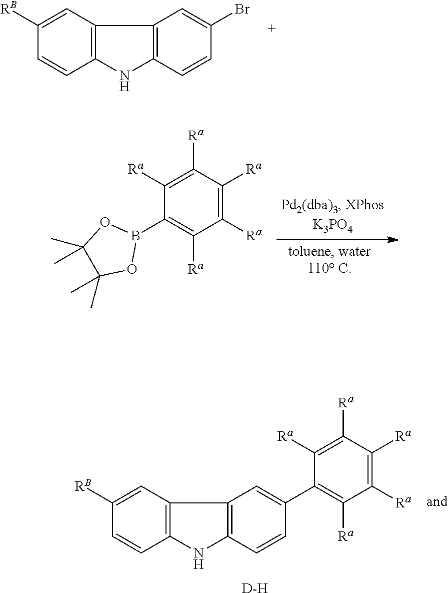

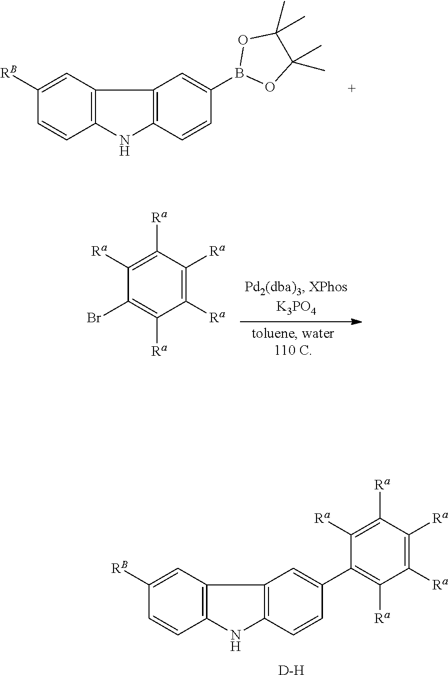

wherein the boronic acid ester can also be replaced by a boronic acid.

Pd₂(dba)₃ (tris(dibenzylideneacetone)dipalladium(0)) is typically used as the palladium catalyst, but alternatives are known to the person skilled in the art. The ligand is selected from S-Phos (2-dicyclohexylphosphino-2′,6′-dimethoxy-1,1′-biphenyl]; or SPhos), X-Phos (2-(dicyclohexylphosphino)-2″,4″,6″-triisopropylbiphenyl or XPhos) and P(Cy)₃ (tricyclohexylphosphine), for example. The salt is selected from potassium phosphate, potassium carbonate, and potassium acetate, for example, and the solvent can be either a pure solvent, such as toluene or dioxane, or a mixture, such as toluene/dioxane/water. The skilled person knows which Pd catalyst, ligand, salt, and solvent combination is most likely to result in high yields.

For the reaction of a nitrogen heterocyclic compound in a nucleophilic aromatic substitution with an aryl halide, preferably an aryl fluoride, typical conditions include the use of a base, such as potassium phosphate tribasic or sodium hydride, for example in an aprotic polar solvent, such as dimethyl sulfoxide (DMSO) or N,N-dimethyl formamide (DMF).

An alternative synthesis pathway includes the introduction of a nitrogen heterocyclic compound via copper- or palladium-catalyzed coupling to an aryl halide or aryl pseudohalide, preferably an aryl bromide, an aryl iodide, aryl triflate, or an aryl tosylate.

A further aspect of the invention relates to the use of an organic molecule according to the invention as a luminescent emitter or as an absorber and/or as a host material and/or as an electron transport material and/or as a hole injection material and/or as a hole blocking material in an optoelectronic device.

The organic electroluminescent device can be understood in the broadest sense to be any device based on organic materials that is suitable for emitting light in the visible or near ultraviolet (UV) range, i.e., in the range of a wavelength from 380 to 800 nm. More preferably, the organic electroluminescent device can be able to emit light in the visible range, i.e., from 400 to 800 nm.

In the context of such a use, the organic optoelectronic device is in particular selected from the group consisting of:
organic light-emitting diodes (OLEDs),
light-emitting electrochemical cells,
OLED sensors, in particular in gas and vapor sensors which are not hermetically shielded to the outside,
organic diodes,
organic solar cells,
organic transistors,
organic field-effect transistors,
organic lasers, and
down-conversion elements.

In a preferred embodiment in the context of such a use, the organic electroluminescent device is a device selected from the group consisting of an organic light-emitting diode (OLED), a light-emitting electrochemical cell (LEC), and a light-emitting transistor.

In the case of said use, the fraction of the organic molecule according to the invention in the emission layer in an optoelectronic device, in particular in OLEDs, is 1 wt % to 99 wt %, in particular 5 wt % to 80 wt %. In an alternative embodiment, the proportion of the organic molecule in the emission layer is 100 wt %.

In one embodiment, the light-emitting layer comprises not only the organic molecules according to the invention but also a host material, the triplet (T1) and singlet (S1) energy levels of which are energetically higher than the triplet (T1) and singlet (S1) energy levels of the organic molecule.

A further aspect of the invention relates to a composition comprising or consisting of:
(a) at least one organic molecule according to the invention, in particular in the form of an emitter and/or a host, and
(b) one or more emitter and/or host materials, which are different from the organic molecule according to the invention, and
(c) optionally one or more dyes and/or one or more solvents.

In one embodiment, the light-emitting layer comprises (or (essentially) consists of) a composition comprising or consisting of:
(a) at least one organic molecule according to the invention, in particular in the form of an emitter and/or a host, and
(b) one or more emitter and/or host materials, which are different from the organic molecule according to the invention, and
(c) optionally one or more dyes and/or one or more solvents.

The light-emitting layer EML particularly preferably comprises (or (essentially) consists of) a composition comprising or consisting of:
(i) 1-50 wt %, preferably 5-40 wt %, in particular 10-30 wt % of one or more organic molecules according to the invention;
(ii) 5-99 wt %, preferably 30-94.9 wt %, in particular 40-89 wt % of the at least one host compound H; and
(iii) optionally 0-94 wt %, preferably 0.1-65 wt %, in particular 1-50 wt % of the at least one further host compound D having a structure that differs from the structure of the molecules according to the invention; and
(iv) optionally 0-94 wt %, preferably 0-65 wt %, in particular 0-50 wt % of a solvent; and
(v) optionally 0-30 wt %, in particular 0-20 wt %, preferably 0-5 wt % of at least one further emitter molecule F having a structure that differs from the structure of the molecules according to the invention.

Energy can preferably be transferred from the host compound H to one or more organic molecules according to the invention, in particular transferred from the first excited triplet state T1(H) of the host compound H to the first excited triplet state T1(E) of the one or more organic molecules according to the invention and/or from the first excited singlet state S1(H) of the host compound H to the first excited singlet state S1(E) of the one or more organic molecules according to the invention.

In a further embodiment, the light-emitting layer EML comprises (or (essentially) consists of) a composition comprising or consisting of:
(i) 1-50 wt %, preferably 5-40 wt %, in particular 10-30 wt % of an organic molecule according to the invention;
(ii) 5-99 wt %, preferably 30-94.9 wt %, in particular 40-89 wt % of a host compound H; and
(iii) optionally 0-94 wt %, preferably 0.1-65 wt %, in particular 1-50 wt % of the at least one further host compound D having a structure that differs from the structure of the molecules according to the invention; and
(iv) optionally 0-94 wt %, preferably 0-65 wt %, in particular 0-50 wt % of a solvent; and
(v) optionally 0-30 wt %, in particular 0-20 wt %, preferably 0-5 wt % of at least one further emitter molecule F having a structure that differs from the structure of the molecules according to the invention.

In one embodiment, the host compound H has a highest occupied molecular orbital HOMO(H) having an energy E^{HOMO}(H) in the range of −5 to −6.5 eV, and the at least one further host compound D has a highest occupied molecular orbital HOMO(D) having an energy E^{HOMO}(D), wherein E^{HOMO}(H)>E^{HOMO}(D).

In a further embodiment, the host compound H has a lowest unoccupied molecular orbital LUMO(H) having an energy E^{LUMO}(H), and the at least one further host compound D has a lowest unoccupied molecular orbital LUMO(D) having an energy E^{LUMO}(D) wherein E^{LUMO}(H)>E^{LUMO}(D).

In one embodiment, the host compound H has a highest occupied molecular orbital HOMO(H) having an energy E^{HOMO}(H) and a lowest unoccupied molecular orbital LUMO(H) having an energy E^{LUMO}(H), and
the at least one further host compound D has a highest occupied molecular orbital HOMO(D) having an energy E^{HOMO}(D) and a lowest unoccupied molecular orbital LUMO(D) having an energy E^{LUMO}(D),
the organic molecule according to the invention has a highest occupied molecular orbital HOMO(D) having an energy E^{HOMO}(E) and a lowest unoccupied molecular orbital LUMO(E) having an energy E^{LUMO}(E),

wherein

E^{HOMO}(H)>E^{HOMO}(D) and the difference between the energy level of the highest occupied molecular orbital HOMO(E) of the organic molecule according to the invention (E^{HOMO}(E)) and the energy level of the highest occupied molecular orbital HOMO(H) of the host compound H (E^{HOMO}(H)) is between −0.5 eV and 0.5 eV, more preferably between −0.3 eV and 0.3 eV; even more preferably between −0.2 eV and 0.2 eV, or even between −0.1 eV and 0.1 eV; and

E^{LUMO}(H)>E^{LUMO}(D) and the difference between the energy level of the lowest unoccupied molecular orbital LUMO(E) of the organic molecule according to the invention (E^{LUMO}(E)) and the lowest unoccupied molecular orbital LUMO(D) of the at least one further host compound D (E^{LUMO}(D)) is between −0.5 eV and 0.5 eV, more preferably between −0.3 eV and 0.3 eV, even more preferably between −0.2 eV and 0.2 eV, or even between −0.1 eV and 0.1 eV.

In a further aspect, the invention relates to an organic optoelectronic device comprising an organic molecule or a composition of the type described here, in particular in the form of a device selected from the group consisting of organic light-emitting diode (OLED), light-emitting electrochemical cell, OLED sensor, in particular gas and vapor sensors which are not hermetically shielded to the outside, organic diode, organic solar cell, organic transistor, organic field-effect transistor, organic laser, and down-conversion element.

In a preferred embodiment, the organic electroluminescent device is a device selected from the group consisting of an organic light-emitting diode (OLED), a light-emitting electrochemical cell (LEC), and a light-emitting transistor.

In one embodiment of the optoelectronic device according to the invention, the organic molecule according to the invention is used as the emission material in a light-emitting layer EML.

In one embodiment of the optoelectronic device according to the invention, the light-emitting layer EML consists of the here described composition according to the invention.

If the organic electroluminescent device is an OLED, it can exhibit the following example of a layer structure:

1. Substrate

2. Anode layer A

3. Hole injection layer, HIL

4. Hole transport layer, HTL

5. Electron blocking layer, EBL

6. Emitting layer, EML

7. Hole blocking layer, HBL

8. Electron transport layer, ETL

9. Electron injection layer, EIL

10. Cathode layer,

wherein the OLED comprises each layer only optionally, various layers can be merged, and the OLED can comprise more than one layer of each layer type defined above.

The organic electroluminescent device can furthermore optionally comprise one or more protective layers, which protect the device from damaging exposure to harmful factors in the environment, including, for example, moisture, steam, and/or gases.

In one embodiment of the invention, the organic electroluminescent device is an OLED comprising the following inverted layer structure:

1. Substrate

2. Cathode layer

3. Electron injection layer, EIL

4. Electron transport layer, ETL

5. Hole blocking layer, HBL

6. Emitting layer, EML

7. Electron blocking layer, EBL

8. Hole transport layer, HTL

9. Hole injection layer, HIL

10. Anode layer A

wherein the OLED with an inverted layer structure comprises each layer only optionally, various layers can be merged, and the OLED can comprise more than one layer of each layer type defined above.

In one embodiment of the invention, the organic electroluminescent device is an OLED, which may exhibit a stacked architecture. Contrary to the typical arrangement in which the OLEDs are positioned side by side, the individual units in this architecture are stacked on top of one another. Blended light can be produced with OLEDs exhibiting a stacked architecture, in particular white light can be produced by stacking blue, green, and red OLEDs. The OLED exhibiting a stacked architecture can furthermore optionally comprise a charge generation layer (CGL) which is typically positioned between two OLED subunits and typically consists of an n-doped and p-doped layer, wherein the n-doped layer of a CGL is typically positioned closer to the anode layer.

In one embodiment of the invention, the organic electroluminescent device is an OLED comprising two or more emission layers between the anode and the cathode. This so-called tandem OLED in particular comprises three emission layers, wherein one emission layer emits red light, one emission layer emits green light, and one emission layer emits blue light, and can optionally comprise further layers, such as charge generation layers, blocking or transport layers between the individual emission layers. In another embodiment, the emission layers are stacked adjacent to one another. In another embodiment, the tandem OLED comprises a charge generation layer between each two emission layers. Adjacent emission layers or emission layers separated by a charge generation layer can also be merged.

The substrate can be formed by any material or any composition of materials. Glass slides are most frequently used as substrates. Alternatively, thin metal layers (e.g., copper, gold, silver, or aluminum foils) or plastic foils or plastic slides can be used. This can allow a higher degree of flexibility. The anode layer A is mostly composed of materials that make it possible to obtain an (essentially) transparent film. Since at least one of the two electrodes should be (essentially) transparent in order to allow light emission from the OLED, either the anode layer A or the cathode layer C is transparent. The anode layer A preferably comprises a large content or even consists of transparent conductive oxides (TCOs). Such an anode layer A can, for example, comprise indium tin oxide, aluminum zinc oxide, fluorine-doped tin oxide, indium zinc oxide, PbO, SnO, zirconium oxide, molybdenum oxide, vanadium oxide, tungsten oxide, graphite, doped Si, doped Ge, doped GaAs, doped polyaniline, doped polypyrrole, and/or doped polythiophene.

The anode layer A particularly preferably (essentially) consists of indium tin oxide (ITO) (e.g., (InO₃)_{0.9}(SnO₂)_{0.1}). The roughness of the anode layer A caused by the transparent conductive oxides (TCOs) can be compensated by using a hole injection layer (HIL). The HIL can also facilitate the injection of quasi-charge carriers (e.g., holes), in which the transport of the quasi-charge carriers from the TCO to the hole transport layer (HTL) is facilitated. The hole injection layer (HIL) can comprise poly-3,4-ethylenedioxythiophene (PEDOT), polystyrene sulfonate (PSS), MoO₂, V₂O₅, CuPC, or CuI, in particular a mixture of PEDOT and PSS. The hole injection layer (HIL) can also prevent the diffusion of metal from the anode layer A into the hole transport layer (HTL). For example, the HIL can comprise PEDOT:PSS (poly-3,4-ethylenedioxythiophene: polystyrene sulfonate), PEDOT (poly-3,4-ethylenedioxythiophene), mMTDATA (4,4′,4″-tris[phenyl(m-tolyl)amino]triphenylamine), Spiro-TAD (2,2′,7,7′-tetrakis(n,n-diphenylamino)-9,9′-spirobifluorene), DNTPD (N1,N1′-(biphenyl-4,4′-diyl)bis(N1-phenyl-N4,N4-di-m-tolylbenzene-1,4-diamine), NPB (N,N′-nis-(1-naphthalenyl)-N,N′-bis-phenyl-(1,1′-biphenyl)-4,4′-diamine), NPNPB (N,N′-diphenyl-N,N′-di-[4-(N,N-diphenyl-amino)phenyl]benzidine), MeO-TPD (N,N,N′,N′-tetrakis(4-methoxyphenyl)benzidine), HAT-CN (1,4,5,8,9,11-hexaazatriphenylene-hexacarbonitrile), and/or Spiro-NPD (N,N′-diphenyl-N,N′-bis-(1-naphthyl)-9,9′-spirobifluorene-2,7-diamine).

Adjacent to the anode layer A or the hole injection layer (HIL), there is typically a hole transport layer (HTL). Any hole transport compound can be used here. Electron-rich heteroaromatic compounds, such as triarylamines and/or carbazoles, for example, can be used as the hole transport compound. The HTL can reduce the energy barrier between the anode layer A and the light-emitting layer EML. The hole transport layer (HTL) can also be an electron blocking layer (EBL). The hole transport compound preferably has high energy levels of its triplet states T1. For example, the hole transport layer (HTL) can comprise a star-shaped heterocyclic compound, such as tris(4-carbazoyl-9-ylphenyl)amine (TCTA), poly-TPD (poly(4-butylphenyl-diphenylamine)), [alpha]-NPD (poly(4-butylphenyl-diphenylamine)), TAPC (4,4′-cyclohexylidene-bis[N,N-bis(4-methylphenyl)benzenamine]), 2-TNATA (4,4′,4″-tris[2-naphthyl(phenyl)amino]triphenylamine), Spiro-TAD, DNTPD, NPB, NPNPB, MeO-TPD, HAT-CN, and/or TrisPcz (9,9′-diphenyl-6-(9-phenyl-9H-carbazole-3-yl)-9H,9′H-3,3′-bicarbazole). The HTL can also comprise a p-doped layer consisting of an inorganic or organic dopant in an organic hole-transporting matrix. Transition metal oxides, such as vanadium oxide, molybdenum oxide, or tungsten oxide, for example, can be used as the inorganic dopant. Tetrafluorotetracyanoquinodimethane (F4-TCNQ), copper pentafluorobenzoate (Cu(I)pFBz), or transition metal complexes, for example, can be used as the organic dopants.

The EBL can comprise mCP (1,3-bis(carbazole-9-yl)benzene), TCTA, 2-TNATA, mCBP (3,3-di(9H-carbazole-9-yl)biphenyl), tris-Pcz, CzSi (9-(4-tert-butylphenyl)-3,6-bis(triphenylsilyl)-9H-carbazole), and/or DCB (N,N′-dicarbazolyl-1,4-dimethylbenzene), for example.

The light-emitting layer EML is typically located adjacent to the hole transport layer (HTL). The light-emitting layer EML comprises at least one light-emitting molecule. The EML in particular comprises at least one light-emitting molecule according to the invention. In one embodiment, the light-emitting layer comprises only the organic molecules according to the invention. The EML typically also comprises one or more host materials. The host material is selected, for example, from CBP (4,4′-bis-(N-carbazolyl)-biphenyl), mCP, mCBP, Sif87 (dibenzo[b,d]thiophene-2-yltriphenylsilane), CzSi, Sif88 (dibenzo[b,d]thiophene-2-yl)diphenylsilane), DPEPO (bis[2-(diphenylphosphino)phenyl] ether oxide), 9-[3-(dibenzofuran-2-yl)phenyl]-9H-carbazole, 9-[3-(dibenzofuran-2-yl)phenyl]-9H-carbazole, 9-[3-(dibenzothiophene-2-yl)phenyl]-9H-carbazole, 9-[3,5-bis(2-dibenzofuranyl)phenyl]-9H-carbazole, 9-[3,5-bis(2-dibenzothiophenyl)phenyl]-9H-carbazole, T2T (2,4,6-tris(biphenyl-3-yl)-1,3,5-triazine), T3T (2,4,6-tris(triphenyl-3-yl)-1,3,5-triazine), and/or TST (2,4,6-tris(9,9′-spirobifluorene-2-yl)-1,3,5-triazine). The host material should typically be selected such that it has first triplet (T1) and first singlet (S1) energy levels that are energetically higher than the first triplet (T1) and first singlet (S1) energy levels of the organic molecule.

In one embodiment of the invention, the EML comprises a so-called mixed-host system with at least one hole-dominant host and an electron-dominant host. In a particular embodiment, the EML comprises exactly one light-emitting molecule according to the invention and a mixed-host system comprising T2T as the electron-dominant host and a host selected from CBP, mCP, mCBP, 9-[3-(dibenzofuran-2-yl)phenyl]-9H-carbazole, 9-[3-(dibenzofuran-2-yl)phenyl]-9H-carbazole, 9-[3-(dibenzothiophene-2-yl)phenyl]-9H-carbazole, 9-[3,5-bis(2-dibenzofuranyl)phenyl]-9H-carbazole, and 9-[3,5-bis(2-dibenzothiophenyl)phenyl]-9H-carbazole as the hole-dominant host. In another embodiment, the EML comprises 50-80 wt %, preferably 60-75 wt % of a host selected from CBP, mCP, mCBP, 9-[3-(dibenzofuran-2-yl)phenyl]-9H-carbazole, 9-[3-(dibenzofuran-2-yl)phenyl]-9H-carbazole, 9-[3-(dibenzothiophene-2-yl)phenyl]-9H-carbazole, 9-[3,5-bis(2-dibenzofuranyl)phenyl]-9H-carbazole, and 9-[3,5-bis(2-dibenzothiophenyl)phenyl]-9H-carbazole; 10-45 wt %, preferably 15-30 wt % T2T and 5-40 wt %, preferably 10-30 wt. %, of the light-emitting molecule according to the invention.

An electron transport layer (ETL) can be located adjacent to the light-emitting layer EML. Any electron transporter can be used here. Electron-poor compounds, such as benzimidazoles, pyridines, triazoles, oxadiazoles (e.g., 1,3,4-oxadiazole), phosphine oxides, and sulfone, can be used, for example. An electron transporter can also be a star-shaped heterocyclic compound, such as 1,3,5-tri(1-phenyl-1H-benzo[d]imidazole-2-yl)phenyl (TPBi). The ETL can comprise NBphen (2,9-bis(naphthalen-2-yl)-4,7-diphenyl-1,10-phenanthroline), Alq3 (Aluminum-tris(8-hydroxyquinoline)), TSPO1 (diphenyl[4-(triphenylsilyl)phenyl]phosphine oxide), BPyTP2 (2,7-di(2,2′-bipyridin-5-yl)triphenyl), Sif87 (dibenzo[b,d]thiophene-2-yltriphenylsilane), Sif88 (dibenzo[b,d]thiophene-2-yl)diphenylsilane), BmPyPhB (1,3-bis[3,5-di(pyridin-3-yl)phenyl]benzene), and/or BTB (4,4′-bis-[2-(4,6-diphenyl-1,3,5-triazinyl)]-1,1′-biphenyl). The ETL can optionally be doped with materials such as Liq. The electron transport layer (ETL) can also block holes, or a hole blocking layer (HBL) is introduced.

The HBL can, for example, comprise BCP (2,9-dimethyl-4,7-diphenyl-1,10-phenanthroline=bathocuproine), BAlq (bis(8-hydroxy-2-methylquinoline)-(4-phenylphenoxy)aluminum), NBphen (2,9-bis(naphthalen-2-yl)-4,7-diphenyl-1,10-phenanthroline), Alq3 (aluminum-tris(8-hydroxyquinoline)), TSPO1 (diphenyl[4-(triphenylsilyl)phenyl]phosphine oxide), T2T (2,4,6-tris(biphenyl-3-yl)-1,3,5-triazine), T3T (2,4,6-tris(triphenyl-3-yl)-1,3,5-triazine), TST (2,4,6-tris(9,9′-spirobifluorene-2-yl)-1,3,5-triazine), and/or TCB/TCP (1,3,5-tris(N-carbazolyl)benzene/1,3,5-tris(carbazole)-9-yl)benzene).

A cathode layer C can be located adjacent to the electron transport layer (ETL). The cathode layer C can, for example, comprise or consist of a metal (e.g., Al, Au, Ag, Pt, Cu, Zn, Ni, Fe, Pb, LiF, Ca, Ba, Mg, In, W, or Pd) or a metal alloy. For practical reasons, the cathode layer can also consist of (essentially) non-transparent metals, such as Mg, Ca, or Al. Alternatively or additionally, the cathode layer C can also comprise graphite and/or carbon nanotubes (CNTs). The cathode layer C can alternatively also consist of nanoscalic silver wires.

An OLED can further optionally comprise a protective layer between the electron transport layer (ETL) and the cathode layer (which can be referred to as an electron injection layer (EIL)). This layer can comprise lithium fluoride, cesium fluoride, silver, Liq (8-hydroxyquinolinolatolithium), Li₂O, BaF₂, MgO, and/or NaF.

The electron transport layer (ETL) and/or a hole blocking layer (HBL) can optionally also comprise one or more host compounds.

In order to modify the emission spectrum and/or the absorption spectrum of the light-emitting layer EML further, the light-emitting layer EML can also comprise one or more further emitter molecules F. Such an emitter molecule F can be any emitter molecule known in the art. Preferably, such an emitter molecule F is a molecule having a structure that differs from the structure of the molecules according to the invention. The emitter molecule F can optionally be a TADF emitter. Alternatively, the emitter molecule F can optionally be a fluorescent and/or phosphorescent emitter molecule capable of changing the emission spectrum and/or the absorption spectrum of the light-emitting layer EML. For example, the triplet and/or singlet excitons can be transferred from the emitter molecule according to the invention to the emitter molecule F before relaxing to the ground state S0 by typically red-shifting light in comparison to the light emitted by emitter molecule E. The emitter molecule F can optionally also provoke two-photon effects (i.e., the absorption of two photons having half the energy of the absorption maximum).

Optionally, an organic electroluminescent device (e.g., an OLED) can, for example, be an essentially white organic electroluminescent device. Such a white organic electroluminescent device can, for example, comprise at least one (deep) blue emitter molecule and one or more emitter molecules that emit green and/or red light. Energy transmittance between two or more molecules can then optionally take place as described above.

As used herein, the designation of the colors of emitted and/or absorbed light is as follows unless defined more specifically in the particular context:

violet: wavelength range of >380-420 nm;

deep blue: wavelength range of >420-480 nm;

sky blue: wavelength range of >480-500 nm;

green: wavelength range of >500-560 nm;

yellow: wavelength range of >560-580 nm;

orange: wavelength range of >580-620 nm;

red: wavelength range of >620-800 nm.

With respect to emitter molecules, said colors relate to the emission maximum. Therefore, for example, a deep blue emitter has an emission maximum in the range of >420 to 480 nm, a sky blue emitter has an emission maximum in the range of >480 to 500 nm, a green emitter has an emission maximum in a range of >500 to 560 nm, a red emitter has an emission maximum in a range of >620 to 800 nm.

A deep blue emitter can preferably have an emission maximum below 480 nm, more preferably below 470 nm, even more preferably below 465 nm, or even below 460 nm. Said maximum is typically above 420 nm, preferably above 430 nm, more preferably above 440 nm, or even above 450 nm.

Accordingly, a further aspect of the present invention relates to an OLED which exhibits an external quantum efficiency at 1000 cd/m² of more than 8%, more preferably more than 10%, more preferably more than 13%, even more preferably more than 15%, or even more than 20% and/or exhibits an emission maximum between 420 nm and 500 nm, preferably between 430 nm and 490 nm, more preferably between 440 nm and 480 nm, even more preferably between 450 nm and 470 nm and/or exhibits an LT80 value at 500 cd/m² of more than 100 h, preferably more than 200 h, more preferably more than 400 h, even more preferably more than 750 h, or even more than 1000 h. Accordingly, a further aspect of the present invention relates to an OLED, the emission of which exhibits a CIEy color coordinate of less than 0.45, preferably less than 0.30, more preferably less than 0.20, or even more preferably less than 0.15, or even less than 0.10.

A further aspect of the present invention relates to an OLED which emits light at a distinct color point. According to the present invention, the OLED emits light with a narrow emission range (small full width at half maximum (FWHM)). In one aspect, the OLED according to the invention emits light with a FWHM of the main emission peak of less than 0.50 eV, preferably less than 0.48 eV, more preferably less than 0.45 eV, even more preferably less than 0.43 eV, or less than 0.40 eV.

A further aspect of the present invention relates to an OLED which emits light with CIEx and CIEy color coordinates close to the CIEx (=0.131) and CIEy (=0.046) color coordinates of the primary color blue (CIEx=0.131 and CIEy=0.046) as defined by ITU-R Recommendation BT.2020 (Rec. 2020), and is thus suited for use in ultra-high definition (UHD) displays, e.g., UHD TVs. In traditional applications, top-emitting (top electrode is transparent) devices are typically used, whereas test devices, such as those used throughout the description, are bottom-emitting devices (bottom electrode and substrate are transparent). The CIEy color coordinate of a blue device can be reduced by up to a factor of two, when changing from a bottom- to a top-emitting device, while the CIEx remains nearly unchanged (Okinaka et al. (2015), 22.1: Invited Paper: New Fluorescent Blue Host Materials for Achieving Low Voltage in OLEDs, SID Symposium Digest of Technical Papers, 46; doi:10.1002/sdtp.10480). Accordingly, a further aspect of the present invention relates to an OLED, the emission of which exhibits a CIEx color coordinate between 0.02 and 0.30, preferably between 0.03 and 0.25, more preferably between 0.05 and 0.20, or more preferably between 0.08 and 0.18, or even between 0.10 and 0.15 and/or a CIEy color coordinate between 0.00 and 0.45, preferably between 0.01 and 0.30, more preferably between 0.02 and 0.20, or even more preferably between 0.03 and 0.15, or even between 0.04 and 0.10.

In a further aspect, the invention relates to a method for producing an optoelectronic component. In such a case, an organic molecule according to the invention is used.

The organic electroluminescent device, in particular the OLED according to the present invention, can be produced by means of vapor deposition and/or liquid treatment. Accordingly, at least one layer is
produced by means of a sublimation process,
produced by means of an organic vapor phase deposition process,
produced by means of a carrier gas sublimation process,
solution-processed or printed.

The methods used to produce the organic electroluminescent device, in particular the OLED according to the present invention, are known in the art. The various layers are individually and successively deposited on a suitable substrate by means of a subsequent deposition process. The individual layers can be deposited using the same or different deposition methods.

Vapor deposition processes include, for example, thermal (co)evaporation, chemical vapor deposition, and physical vapor deposition. An AMOLED backplane is used as the substrate for an active matrix OLED display. The individual layer can be processed from solutions or dispersions that use adequate solvents. The solution deposition process includes, for example, spin coating, dip coating, and spray printing. Liquid treatment can optionally be carried out in an inert atmosphere (e.g., in a nitrogen atmosphere) and the solvent can optionally be completely or partially removed using means known in the prior art.

### EXAMPLES

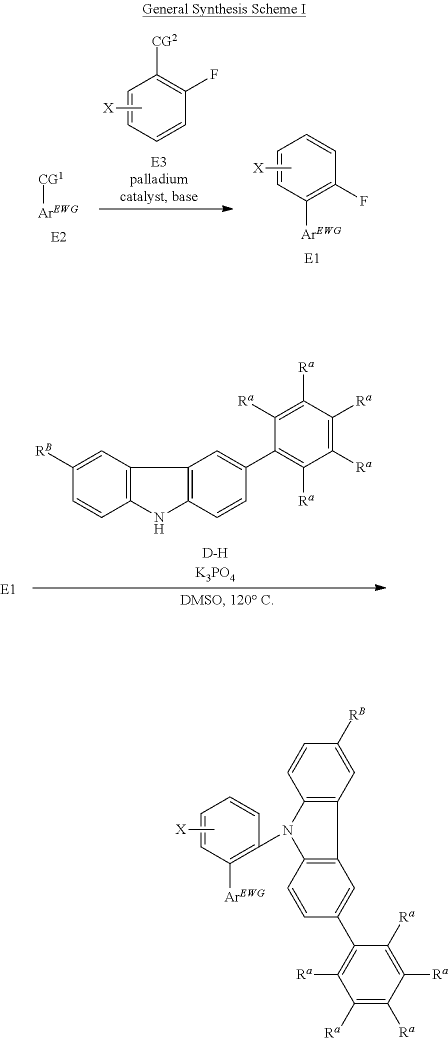

General Procedure for the Synthesis AAV0:

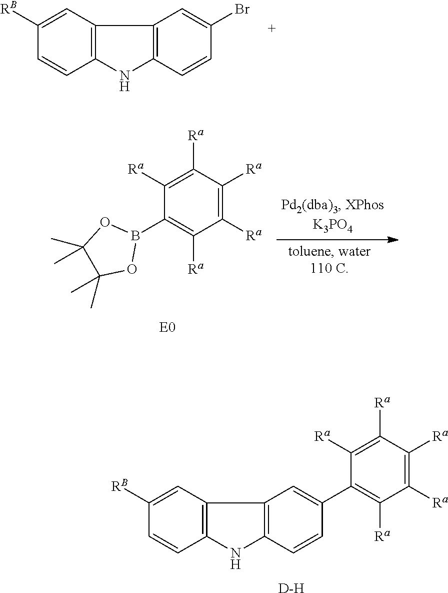

E0 (1.20 equivalents), 6-R^{B}-substituted 3-bromocarbazole (1.00 equivalents), Pd₂(dba)₃ (0.03 equivalents), 2-dicyclohexylphosphino-2′,4′,6′-triisopropylbiphenyl (XPhos) (0.12 equivalents), and potassium phosphate tribasic (1.50 equivalents) are stirred under nitrogen atmosphere in a toluene/water mixture (ratio of 10:1, 3 ml toluene per mmol aryl bromide) at 110° C. for 16 hours. The reaction mixture is then filtered and the residue is washed with dichloromethane. The solvent is removed. The obtained crude product is purified by recrystallization or column chromatography and obtained as a solid.

A corresponding boronic acid can be used instead of a boronic acid ester.

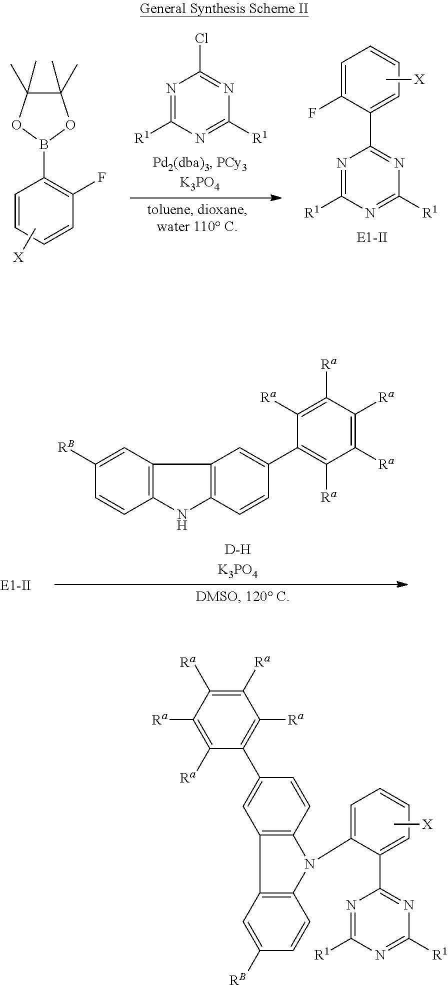

General Procedure for Synthesis AAV1:

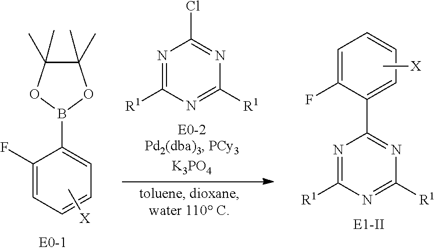

X-substituted fluorophenylboronic acid pinacol ester E0-1 (1.20 equivalents), 2-,4-di-R¹-substituted-6-chloro-1,3,5-triazine E0-2 (1.00 equivalents), Pd₂(dba)₃ (0.03 equivalents), tricyclohexylphosphine (PCy₃) (0.07 equivalents), and potassium phosphate tribasic (2.50 equivalents) are stirred under nitrogen atmosphere in a dioxane/toluene/water mixture (ratio of 4:1:1, 4 ml dioxane per mmol triazine) at 110° C. for 16 hours. The reaction mixture is then filtered and the residue is washed with dichloromethane. The solvent is removed. The obtained crude product is purified by recrystallization or column chromatography and the product E1-II is obtained as a solid.

A corresponding boronic acid can be used instead of a boronic acid ester.

General Procedure for Synthesis AAV2:

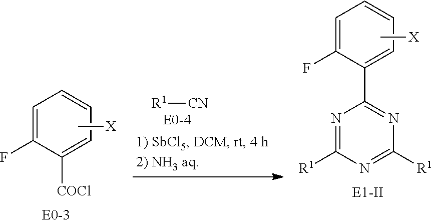

E0-3 (1.00 equivalents) and E0-4 (2.00 equivalents) are dissolved in dichloromethane and cooled in an ice bath. Antimony (V) chloride (1.00 equivalents) is added dropwise to the solution, the reaction mixture is stirred at room temperature for one hour and then stirred at 45° C. for 6 hours. The intermediate product is filtered and washed with dichloromethane.

The dried solid is added to a cooled 25% ammonia solution (0-5° C.) and stirred overnight at room temperature. The reaction mixture is filtered and the obtained solid is washed with water. The solid is added to DMF and stirred at 155° C. for 30 minutes. The precipitated solid is filtered hot. Water is added to the hot DMF solution to precipitate the product. The product E1-II is obtained by filtration.

General Procedure for Synthesis AAV3

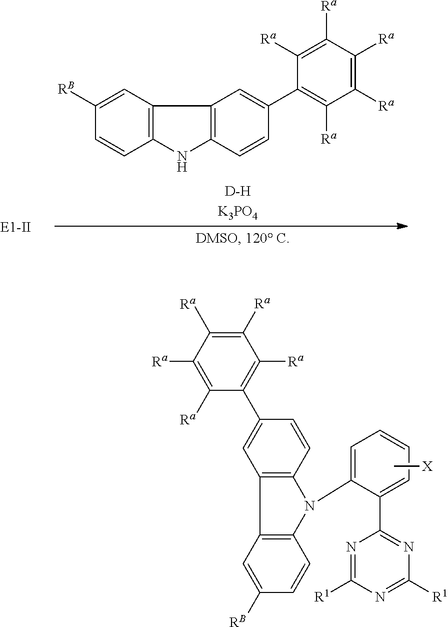

E1-II (1.00 equivalents), the corresponding donor molecule D-H (1.00 equivalents), and potassium phosphate tribasic (4.00 equivalents) are suspended in DMSO under nitrogen atmosphere and stirred at 120° C. (16 h). The reaction mixture is then added to saturated sodium chloride solution and extracted three times with dichloromethane. The combined organic phases are washed twice with saturated sodium chloride solution, dried over MgSO₄, and the solvent is removed. The crude product is purified by recrystallization or by flash chromatography. The product is obtained as a solid.

Alternatively, halogen-substituted carbazole D-H-Hal, for example, in particular a 3-bromo-substituted carbazole, for example 3-bromocarbazole, can be used in AAV3 instead of D-H. In a subsequent reaction, a boronic acid ester group or a boronic acid group can, for example, be introduced at the position of one or more halogen substituents introduced via D-H-Hal, and thus produce the corresponding carbazole-3-ylboronic acid ester or the corresponding carbazole-3-boronic acid, for example by the reaction with bis(pinacol)diboronic acid (CAS No. 73183-34-3). A substituent

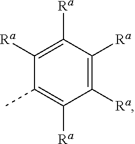

wherein the dashed line represents the binding site, can then be introduced instead of the boronic acid ester group or the boronic acid group via a coupling reaction with the corresponding halogenated reactant, preferably

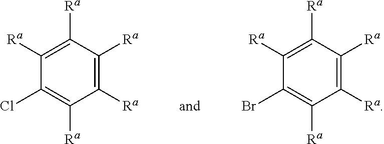

Alternatively, a substituent

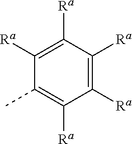

can be introduced at the position of the one halogen substituent, which was introduced via D-H-Hal, via the reaction with a boronic acid of the substituent

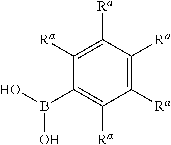

or a corresponding boronic acid ester.

The bromination of a carbazole derivative can alternatively be produced in a reaction with N-bromosuccinimide, for example as shown in the following reaction scheme:

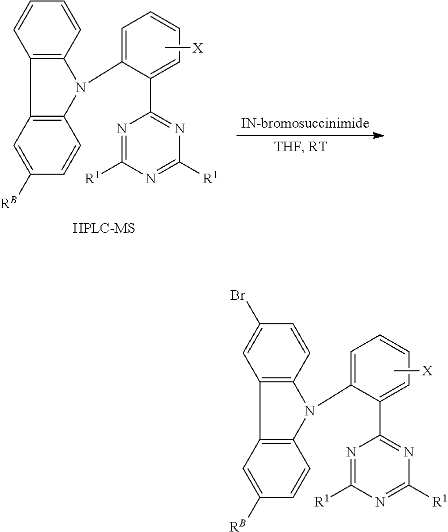

HPLC-MS spectroscopy is carried out on an HPLC system of the company Agilent (1100 series) with an MS detector (Thermo LTQ XL).

A reversed-phase column 4.6 mm×150 mm, particle size 3.5 μm of the company Agilent (ZORBAX Eclipse Plus 95 Å C18, 4.6×150 mm, 3.5 μm HPLC column). for example, is used in the HPLC. The HPLC-MS measurements are carried out at room temperature (RT) with the following gradients:

| Flow rate [ml/min] | Time [min] | A [%] | B [%] | C [%] |
|---|---|---|---|---|
| 2.5 | 0 | 40 | 50 | 10 |
| 2.5 | 5 | 40 | 50 | 10 |
| 2.5 | 25 | 10 | 20 | 70 |
| 2.5 | 35 | 10 | 20 | 70 |

wherein the following solvent mixtures are used:

| | | |
|---|---|---|
| Solvent A: | H₂O (90%) | MeCN (10%) |
| Solvent B: | H₂O (10%) | MeCN (90%) |
| Solvent C: | THF (50%) | MeCN (50%) |

For the measurements, an injection volume of 5 μl is taken from a solution with a concentration of 0.5 mg/ml.

The sample is ionized by means of APCI (chemical ionization at atmospheric pressure) either in positive (APCI+) or negative (APCI−) ionization mode.

Cyclic Voltammetry

Cyclic voltammograms are measured using solutions having a concentration of 10³ mol/l of the organic molecules in dichloromethane or a suitable solvent and a suitable supporting electrolyte (e.g., 0.1 mmol/l of tetrabutylammonium hexafluorophosphate). The measurements are conducted at room temperature under nitrogen atmosphere with a three-electrode assembly (working and counter electrodes: Pt wire, reference electrode: Pt wire) and calibrated using FeCp₂/FeCp₂⁺ as the internal standard. The HOMO data were corrected using ferrocene as the internal standard against a saturated calomel electrode (SCE).

Calculation Based on Density Functional Theory

Molecular structures are optimized using the BP68 functional and the resolution of identity approach (RI). Excitation energies are calculated using the (BP68) optimized structures using time-dependent DFT (TD-DFT) structures. Orbital and excited state energies are calculated with the B2LYP functional. Def2-SVP basis sets and an m4 grid for numerical integration are used. The Turbomole program package is used for all calculations.

Photophysical Measurements

Sample pretreatment: spin coating

Device: Spin150, SPS Euro.

The sample concentration is 1 mg/ml, prepared in chloroform or dichloromethane. 9 mg PMMA are added to 1 ml of the corresponding solution. The corresponding thin film is produced with 50 μl of the resulting solution.

Program: 1) 3 s at 400 rpm; 2) 20 s at 1000 rpm at 1000 rpm/s; 3) 10 s at 4000 rpm at 1000 rpm/s. After coating, the films were dried on a LHG precision heating plate for 1 min at 70° C. in air.

Photoluminescence Spectroscopy and TCSPC (Time-Correlated Single-Photon Counting)

Stationary emission spectroscopy was carried out using a fluorescence spectrometer of the Horiba Scientific company, Model Fluoromax-4, equipped with a 150 W xenon arc lamp, excitation and emission monochromators, and a Hamamatsu R928 photomultiplier tube, as well as a time-correlated single-photon counting (TCSPC) option. The sample chamber was continuously flushed with nitrogen (flow rate>800 ml/min). Emission and excitation spectra were corrected using standard correction curves.

Emission decay times are determined using the same system using the TCSPC method with FM-2013 equipment and a Horiba Yvon TCSPC Hub.

Excitation Sources:

NanoLED 370 (wavelength: 371 nm, pulse duration: 1.1 ns)

NanoLED 290 (wavelength: 294 nm, pulse duration: <1 ns)

SpectraLED 310 (wavelength: 314 nm)

SpectraLED 355 (wavelength: 355 nm).

The analysis (exponential fitting) was performed using the DataStation software package and the DAS6 analysis software. The fit was specified with the aid of the chi square method

$${c}^{2} = {\sum }_{k = 1}^{i} \frac{{( {e}_{i} - {o}_{i} )}^{2}}{{e}_{i}}$$

where eᵢ: variable predicted by the fit and oᵢ: measured variable.

Time-Resolved PL Spectroscopy in the μs Range

Time-resolved transients are furthermore measured on an Edinburgh Instruments FS5 fluorescence spectrometer. Compared to measurements on the comparable HORIBA system, the FS5 allows better light utilization and consequently an improved ratio between sample emission and background noise, which improves measurements of delayed fluorescent emitters in particular. The sample to be examined is excited by a broadband xenon lamp (150 W xenon arc lamp). The FS5 uses Czerny-Turner monochromators for both selective excitation wavelengths and emission wavelengths. The photoluminescence of the sample is detected via an R928P photomultiplier tube, wherein the photocathode of the detector enables time-resolved measurement in the spectral range from 200 nm to 870 nm. The temperature-stabilized detector unit also ensures a dark count rate of less than 300 events per second. In order to determine the decay time of the PL transient, the μs range is subsequently approximated using three exponential functions. Via amplitude weighting, an averaged lifetime τ_{DF} for the delayed fluorescence is obtained from

$${τ}_{DF} = {\sum }_{i = 1}^{3} \frac{{A}_{i} {τ}_{i}}{{A}_{i}} ,$$

with the respective monoexponential decay times τᵢ and associated amplitudes Aᵢ.

Measurements of the Photoluminescence Quantum Yield

The measurement of the photoluminescence quantum yield (PLQY) was carried out by means of an Absolute PL Quantum Yield Measurement C9920-03G system of the company Hamamatsu Photonics. Said system consists of a 150 W xenon gas discharge lamp, automatically adjustable Czerny-Turner monochromators (250-950 nm) and an Ulbricht sphere with a high reflectance Spectralon coating (a Teflon derivative), which is connected via a fiber optic cable to a PMA-12 multichannel detector with a BT (back-thinned)-CCD chip having 1024×122 pixels (size 24×24 μm). The quantum efficiency and the CIE coordinates were analyzed using the software U6039-05 Version 3.6.0. Emission maxima are stated in nm, quantum yields 0 in %, and CIE coordinates as x, y values.

PLQY is determined using the following protocol:
1) Implementation of quality assurance measures: Anthracene in ethanol at a known concentration is used as the reference material.
2) Determination of the excitation wavelength: The absorption maximum of the organic molecule was determined first and excitation was carried out therewith.
3) Implementation of the sample measurement:

The absolute quantum yield of degassed solutions and films was determined under a nitrogen atmosphere.

The calculation was performed within the system according to the following equation:

$${Φ}_{PL} = \frac{{n}_{photon} , emitted}{{n}_{photon} , absorbed} = \frac{\int \frac{λ}{hc} [ {Int}_{emitted}^{sample} ( λ ) - {Int}_{emitted}^{reference} ( λ ) ] d λ}{\int \frac{λ}{hc} [ {Int}_{emitted}^{reference} - {Int}_{absorbed}^{sample} ( λ ) ] d λ}$$

wherein nₚₕₒₜₒₙ denotes the photon count and Int. denotes the intensity.

Production and Characterization of Organic Electroluminescent Devices OLED devices comprising organic molecules according to the invention can be produced using vacuum deposition methods. If a layer contains more than one compound, the weight percentage of one or more compounds is stated in %. The total weight percentage values amount to 100%, therefore if a value is not provided, the fraction of that compound is equal to the difference between the provided values and 100%.

The not fully optimized OLEDs are characterized using standard methods and the measurement of electroluminescence spectra, the external quantum efficiency (in %) as a function of the intensity, calculated using the light detected by the photodiode and the current. The lifetime of the OLED device is extracted from the change of the luminance during operation at constant current density.

The LT50 value corresponds to the time at which the measured luminance has dropped to 50% of the initial luminance, LT80 analogously corresponds to the time at which the measured luminance has dropped to 80% of the initial luminance, LT95 to the time at which the measured luminance has dropped to 95% of the initial luminance, etc.

Accelerated measurements of the lifetime are carried out (e.g., applying increased current densities). LT80 values at 500 cd/m², for example, are determined using the following equation:

$$L T 80 ( 500 \frac{{cd}^{2}}{{m}^{2}} ) = L T 80 ( {L}_{0} ) {( \frac{{L}_{0}}{500 \frac{{cd}^{2}}{{m}^{2}}} )}^{1.6}$$

wherein L₀ denotes the initial luminance at the applied current density.

The value corresponds to the average of several pixels (typically two to eight), wherein the standard deviation between said pixels is given. The figures show the data series for one OLED pixel.

### Example 1

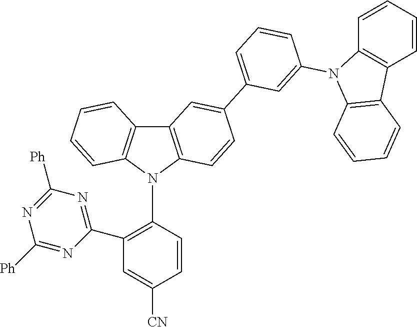 [SMILES: N#Cc1ccc(-n2c3ccccc3c3cc(-c4cccc(-n5c6ccccc6c6ccccc65)c4)ccc32)c(-c2nc(-c3ccccc3)nc(-c3ccccc3)n2)c1]

Example 1 was synthesized according to

AAV0 (74% yield), wherein 3-bromocarbazole (CAS 1592-95-6) and

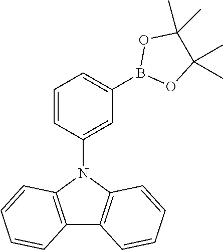 [SMILES: CC1(C)OB(c2cccc(-n3c4ccccc4c4ccccc43)c2)OC1(C)C]

were used as E0,

AAV1 (83% yield), wherein 2-chloro-4,6-diphenyl-1,3,5-triazine (CAS 3842-55-5) was used as E0-2 and 2-fluoro-5-cyanophenylboronic acid pinacol ester was used as E0-1, and

AAV3 (68% yield).

MS (HPLC-MS), m/z (retention time): 740.24 (11.71 min).

FIG. 1 shows the emission spectrum of Example 1 (10 wt % in PMMA). The emission maximum is at 483 nm. The photoluminescence quantum yield (PLQY) is 82%, the full width at half maximum is 0.43 eV and the emission decay time is 10 μs.

### Example 2

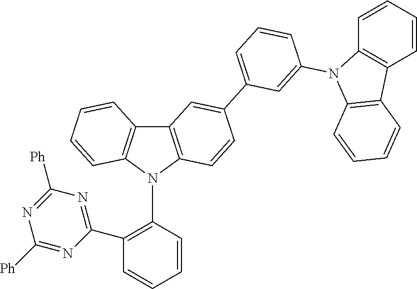 [SMILES: c1ccc(-c2nc(-c3ccccc3)nc(-c3ccccc3-n3c4ccccc4c4cc(-c5cccc(-n6c7ccccc7c7ccccc76)c5)ccc43)n2)cc1]

Example 2 was synthesized according to AAV0 (74% yield), wherein 3-bromocarbazole (CAS 1592-95-6) and

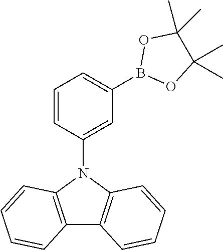 [SMILES: CC1(C)OB(c2cccc(-n3c4ccccc4c4ccccc43)c2)OC1(C)C]

were used as E0,

AAV2 (99% yield), wherein benzonitrile (CAS 100-47-0) was used as E0-4 and 2-fluorobenzoyl chloride was used as E0-3, and AAV3 (50% yield).

MS (HPLC-MS), m/z (retention time): 714.23 (12.75 min).

FIG. 2 shows the emission spectrum of Example 2 (10 wt % in PMMA). The emission maximum is at 467 nm. The photoluminescence quantum yield (PLQY) is 75%, the full width at half maximum is 0.43 eV and the emission decay time is 19 μs.

### Example 3

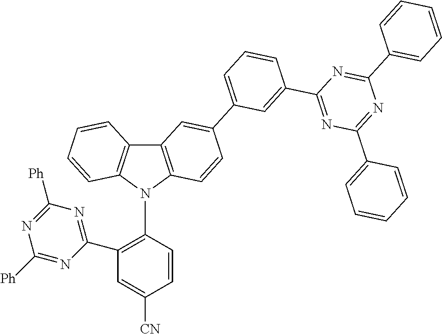 [SMILES: N#Cc1ccc(-n2c3ccccc3c3cc(-c4cccc(-c5nc(-c6ccccc6)nc(-c6ccccc6)n5)c4)ccc32)c(-c2nc(-c3ccccc3)nc(-c3ccccc3)n2)c1]

Example 3 was synthesized according to

AAV0 (27% yield), wherein 3-bromocarbazole (CAS 1592-95-6) and

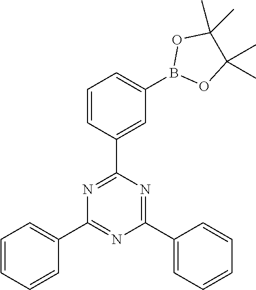 [SMILES: CC1(C)OB(c2cccc(-c3nc(-c4ccccc4)nc(-c4ccccc4)n3)c2)OC1(C)C]

were used as E0,

AAV1 (83% yield), wherein 2-chloro-4,6-diphenyl-1,3,5-triazine (CAS 3842-55-5) was used as E0-2 and 2-fluoro-5-cyanophenylboronic acid pinacol ester was used as E0-1, and AAV3 (65% yield).

MS (HPLC-MS), m/z (retention time): 806.24 (12.43 min).

FIG. 3 shows the emission spectrum of Example 3 (10 wt % in PMMA). The emission maximum is at 490 nm. The photoluminescence quantum yield (PLQY) is 74% and the full width at half maximum is 0.44 eV.

### Example 4

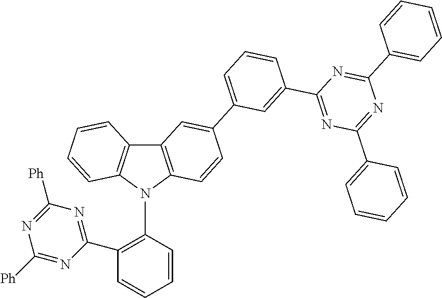 [SMILES: c1ccc(-c2nc(-c3ccccc3)nc(-c3cccc(-c4ccc5c(c4)c4ccccc4n5-c4ccccc4-c4nc(-c5ccccc5)nc(-c5ccccc5)n4)c3)n2)cc1]

Example 4 was synthesized according to

AAV0 (27% yield), wherein 3-bromocarbazole (CAS 1592-95-6) and

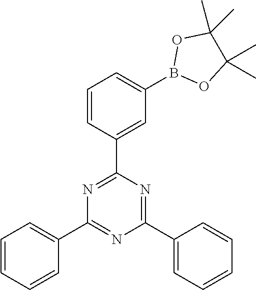 [SMILES: CC1(C)OB(c2cccc(-c3nc(-c4ccccc4)nc(-c4ccccc4)n3)c2)OC1(C)C]

were used as E0,

AAV2 (99% yield), wherein benzonitrile (CAS 100-47-0) was used as E0-4 and 2-fluorobenzoyl chloride was used as E0-3, and

AAV3 (74% yield).

MS (HPLC-MS), m/z (retention time): 780.19 (17.13 min).

FIG. 4 shows the emission spectrum of Example 4 (10 wt % in PMMA). The emission maximum is at 473 nm. The photoluminescence quantum yield (PLQY) is 62%, the full width at half maximum is 0.45 eV and the emission decay time is 43 μs.

### Example 5

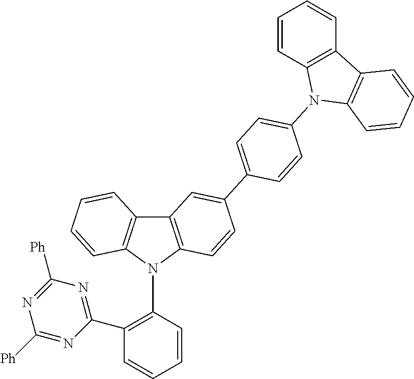 [SMILES: c1ccc(-c2nc(-c3ccccc3)nc(-c3ccccc3-n3c4ccccc4c4cc(-c5ccc(-n6c7ccccc7c7ccccc76)cc5)ccc43)n2)cc1]

Example 5 was synthesized according to

AAV0 (62% yield), wherein 3-bromocarbazole (CAS 1592-95-6) and

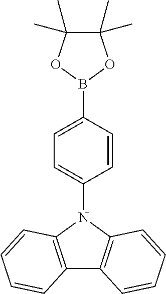 [SMILES: CC1(C)OB(c2ccc(-n3c4ccccc4c4ccccc43)cc2)OC1(C)C]

were used as E0,

AAV2 (99% yield), wherein benzonitrile (CAS 100-47-0) was used as E0-4 and 2-fluorobenzoyl chloride was used as E0-3, and AAV3 (31% yield).

MS (HPLC-MS), m/z (retention time): 716.36 (15.17 min).

FIG. 5 shows the emission spectrum of Example 5 (10 wt % in PMMA). The emission maximum is at 473 nm. The photoluminescence quantum yield (PLQY) is 77%, the full width at half maximum is 0.44 eV and the emission decay time is 60 μs.

### Example 6

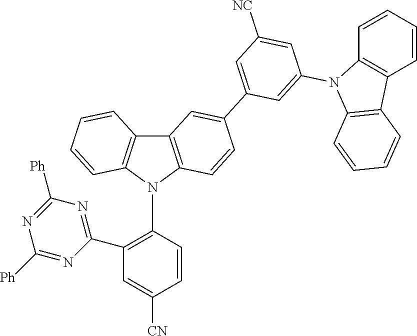 [SMILES: N#Cc1cc(-c2ccc3c(c2)c2ccccc2n3-c2ccc(C#N)cc2-c2nc(-c3ccccc3)nc(-c3ccccc3)n2)cc(-n2c3ccccc3c3ccccc32)c1]

Example 6 was synthesized according to

AAV1 (83% yield), wherein 2-chloro-4,6-diphenyl-1,3,5-triazine (CAS 3842-55-5) was used as E0-2 and 2-fluoro-5-cyanophenylboronic acid pinacol ester was used as E0-1, analogously to AAV3 (81% yield), wherein 3-bromocarbazole (CAS 1592-95-6) was used as D-H. The product from the reaction according to AAV3:

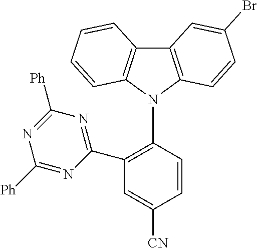 [SMILES: N#Cc1ccc(-n2c3ccccc3c3cc(Br)ccc32)c(-c2nc(-c3ccccc3)nc(-c3ccccc3)n2)c1]

is then reacted with bis(pinacolato)diboron as follows:

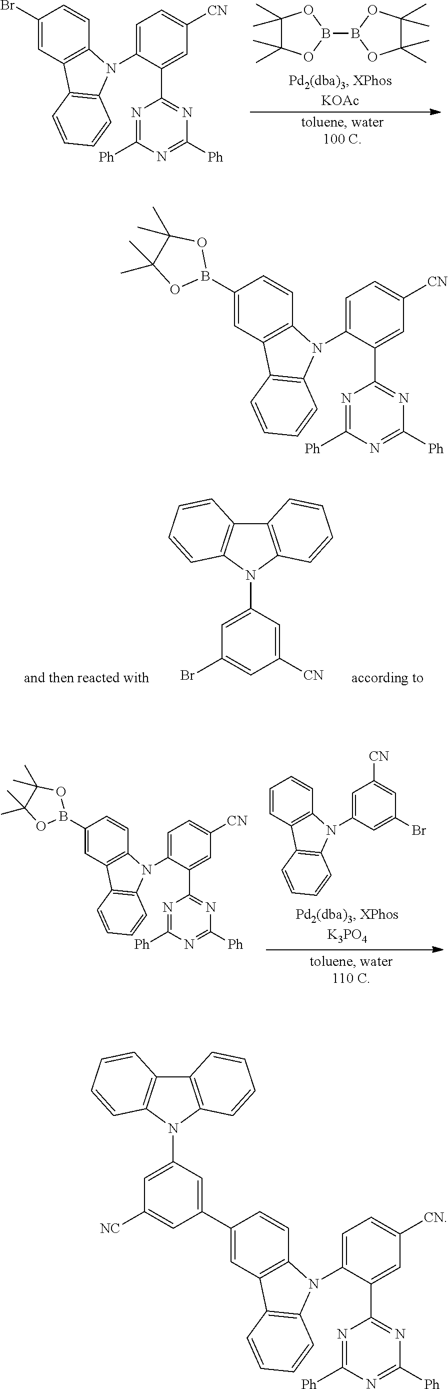

MS (HPLC-MS), m/z (retention time): 765.22 (11.07 min).

FIG. 6 shows the emission spectrum of Example 6 (10 wt % in PMMA). The emission maximum is at 473 nm. The photoluminescence quantum yield (PLQY) is 64%, the full width at half maximum is 0.43 eV and the emission decay time is 15 μs.

### Example 7

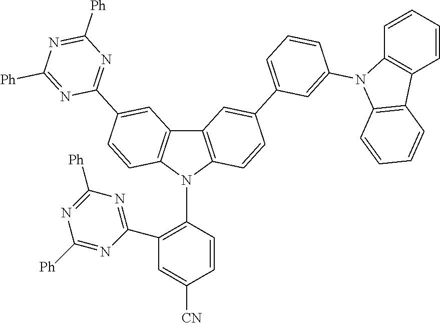 [SMILES: N#Cc1ccc(-n2c3ccc(-c4cccc(-n5c6ccccc6c6ccccc65)c4)cc3c3cc(-c4nc(-c5ccccc5)nc(-c5ccccc5)n4)ccc32)c(-c2nc(-c3ccccc3)nc(-c3ccccc3)n2)c1]

Example 7 was synthesized according to AAV2 (99% yield), wherein benzonitrile (CAS 100-47-0) was used as E0-4 and 2-fluorobenzoyl chloride was used as E0-3, analogously to AAV3 (81% yield), wherein 3-bromocarbazole (CAS 1592-95-6) was used as D-H. The product from the reaction according to AAV3:

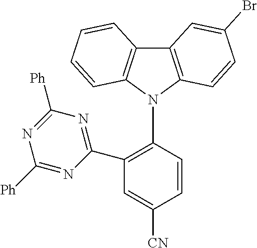 [SMILES: N#Cc1ccc(-n2c3ccccc3c3cc(Br)ccc32)c(-c2nc(-c3ccccc3)nc(-c3ccccc3)n2)c1]

was then reacted with bis(pinacolato)diboron as follows:

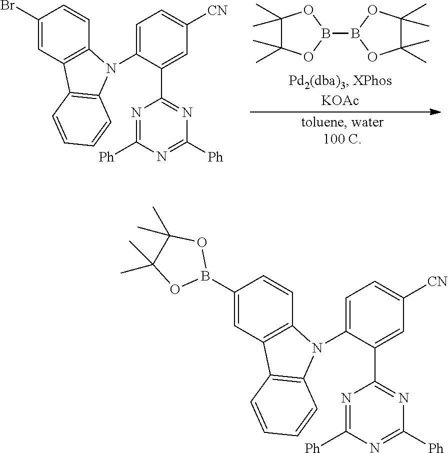

and then reacted with 2-chloro-4,6-diphenyl-1,3,5-triazine (CAS 3842-55-5) according to

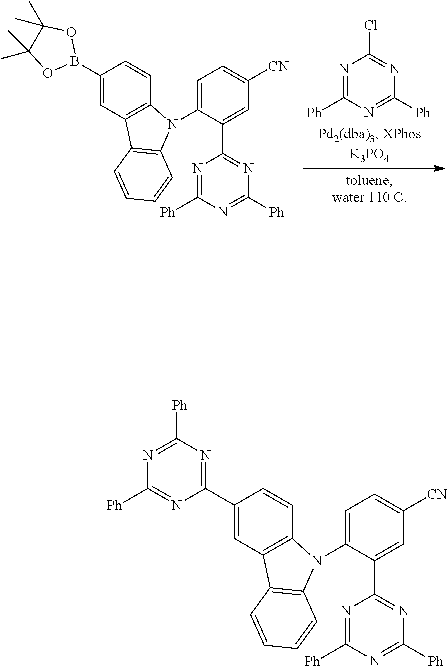 [SMILES: CC1(C)OB(c2ccc3c(c2)c2ccccc2n3-c2ccc(C#N)cc2-c2nc(-c3ccccc3)nc(-c3ccccc3)n2)OC1(C)C ; Clc1nc(-c2ccccc2)nc(-c2ccccc2)n1 ; N#Cc1ccc(-n2c3ccccc3c3cc(-c4nc(-c5ccccc5)nc(-c5ccccc5)n4)ccc32)c(-c2nc(-c3ccccc3)nc(-c3ccccc3)n2)c1]

which was then reacted with N-bromosuccinimide (CAS 128-08-5) according to

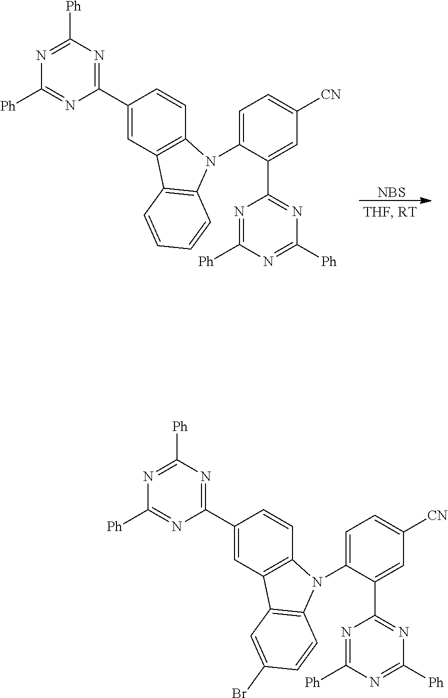 [SMILES: N#Cc1ccc(-n2c3ccccc3c3cc(-c4nc(-c5ccccc5)nc(-c5ccccc5)n4)ccc32)c(-c2nc(-c3ccccc3)nc(-c3ccccc3)n2)c1 ; "NBS" ; N#Cc1ccc(-n2c3ccc(Br)cc3c3cc(-c4nc(-c5ccccc5)nc(-c5ccccc5)n4)ccc32)c(-c2nc(-c3ccccc3)nc(-c3ccccc3)n2)c1]

(83% yield). The product from the reaction was reacted according to

AAV0 (68% yield), wherein

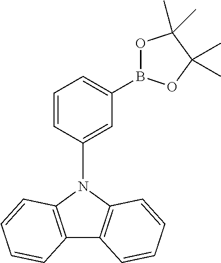 [SMILES: CC1(C)OB(c2cccc(-n3c4ccccc4c4ccccc43)c2)OC1(C)C]

was used as E0.

MS (HPLC-MS), m/z (retention time): 971.17 (15.43 min).

FIG. 7 shows the emission spectrum of Example 7 (10 wt % in PMMA). The emission maximum is at 481 nm. The photoluminescence quantum yield (PLQY) is 69%, the full width at half maximum is 0.43 eV and the emission decay time is 12 μs.

### Example 8

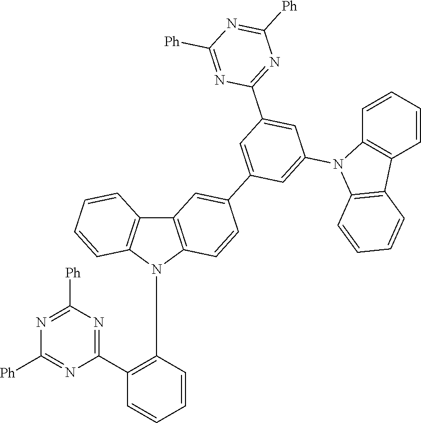 [SMILES: c1ccc(-c2nc(-c3ccccc3)nc(-c3cc(-c4ccc5c(c4)c4ccccc4n5-c4ccccc4-c4nc(-c5ccccc5)nc(-c5ccccc5)n4)cc(-n4c5ccccc5c5ccccc54)c3)n2)cc1]

The emission maximum of Example 8 (10 wt % in PMMA) is at 466 nm.

### Example 9

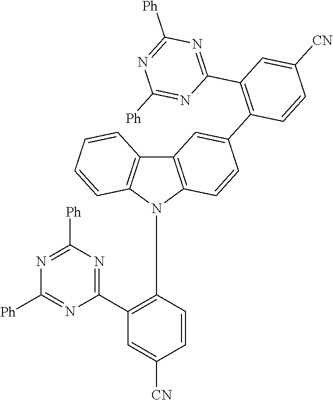 [SMILES: N#Cc1ccc(-c2ccc3c(c2)c2ccccc2n3-c2ccc(C#N)cc2-c2nc(-c3ccccc3)nc(-c3ccccc3)n2)c(-c2nc(-c3ccccc3)nc(-c3ccccc3)n2)c1]

The emission maximum of Example 9 (10 wt % in PMMA) is at 480 nm.

### Examples D1

Example 1 was tested in the OLED components D1 with the following structure (the fraction of the molecule according to the invention in the emission layer is stated in percent by mass):

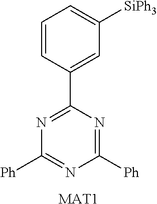

| Layer | Thickness | D1 |
|---|---|---|
| 10 | 100 nm | Al |
| 9 | 2 nm | Liq |
| 8 | 20 nm | NBPhen |
| 7 | 10 nm | MAT1 |
| 6 | 50 nm | mCBP (70%): |
| | | MAT1 (20%): |
| | | 1 (10%) |
| 5 | 10 nm | mCBP |
| 4 | 10 nm | TCTA |
| 3 | 40 nm | NPB |
| 2 | 5 nm | HAT-CN |
| 1 | 50 nm | ITO |
| Substrate | | Glass |

An external quantum efficiency at 1000 cd/m² of 19.2%±0.7 was determined for the component D1. The emission maximum is at 487 nm, CIEx was determined with 0.32 and CIEy with 0.17 at 5.9 V.

Other Examples of Organic Molecules According to the Invention

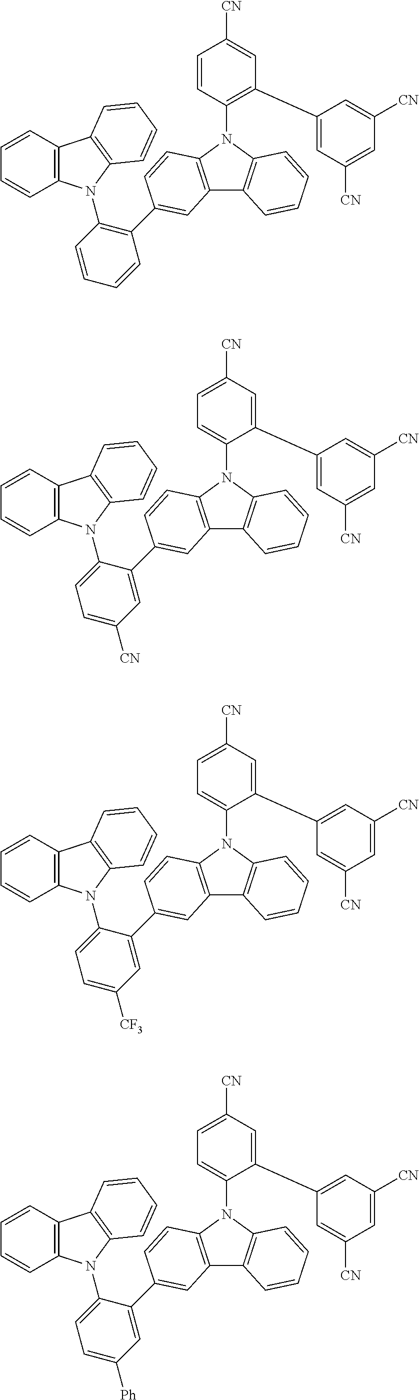 [SMILES: N#Cc1cc(C#N)cc(-c2cc(C#N)ccc2-n2c3ccccc3c3cc(-c4ccccc4-n4c5ccccc5c5ccccc54)ccc32)c1 ; N#Cc1cc(C#N)cc(-c2cc(C#N)ccc2-n2c3ccccc3c3cc(-c4cc(C#N)ccc4-n4c5ccccc5c5ccccc54)ccc32)c1 ; N#Cc1cc(C#N)cc(-c2cc(C#N)ccc2-n2c3ccccc3c3cc(-c4cc(C(F)(F)F)ccc4-n4c5ccccc5c5ccccc54)ccc32)c1 ; N#Cc1cc(C#N)cc(-c2cc(C#N)ccc2-n2c3ccccc3c3cc(-c4cc(-c5ccccc5)ccc4-n4c5ccccc5c5ccccc54)ccc32)c1]

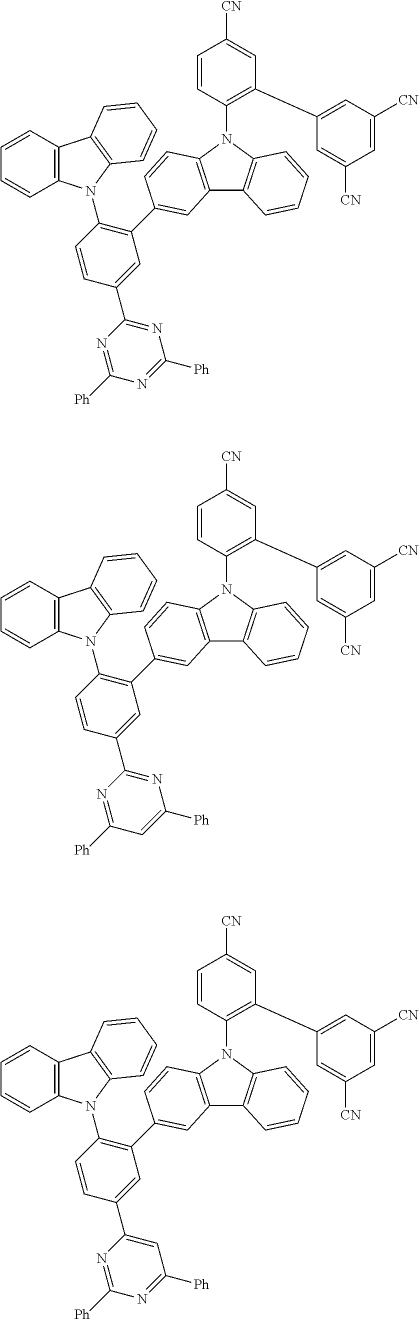 [SMILES: N#Cc1cc(C#N)cc(-c2cc(C#N)ccc2-n2c3ccccc3c3cc(-c4cc(-c5nc(-c6ccccc6)nc(-c6ccccc6)n5)ccc4-n4c5ccccc5c5ccccc54)ccc32)c1 ; N#Cc1cc(C#N)cc(-c2cc(C#N)ccc2-n2c3ccccc3c3cc(-c4cc(-c5nc(-c6ccccc6)cc(-c6ccccc6)n5)ccc4-n4c5ccccc5c5ccccc54)ccc32)c1 ; N#Cc1cc(C#N)cc(-c2cc(C#N)ccc2-n2c3ccccc3c3cc(-c4cc(-c5cc(-c6ccccc6)nc(-c6ccccc6)n5)ccc4-n4c5ccccc5c5ccccc54)ccc32)c1]

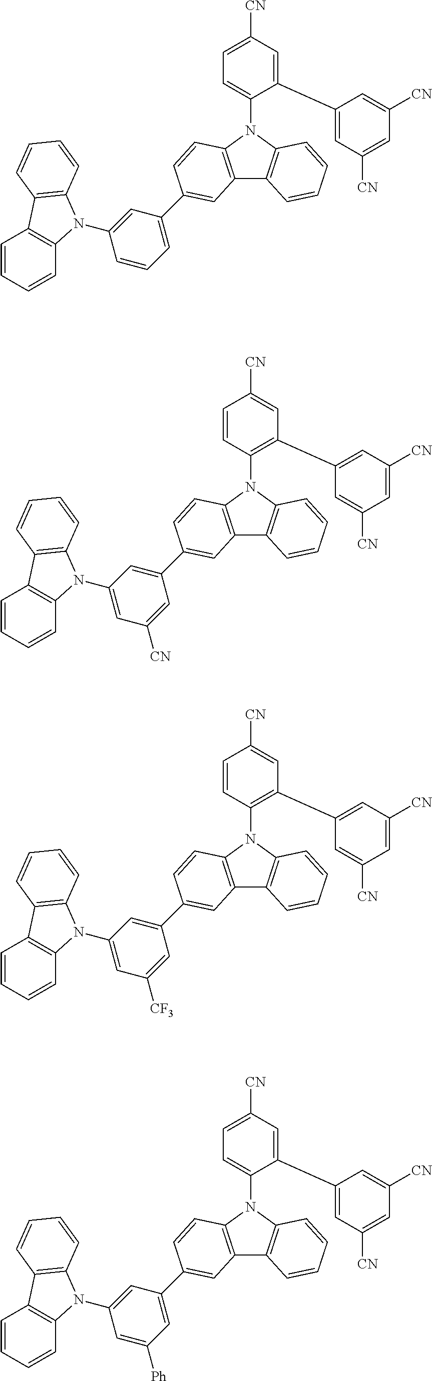 [SMILES: N#Cc1cc(C#N)cc(-c2cc(C#N)ccc2-n2c3ccccc3c3cc(-c4cccc(-n5c6ccccc6c6ccccc65)c4)ccc32)c1 ; N#Cc1cc(C#N)cc(-c2cc(C#N)ccc2-n2c3ccccc3c3cc(-c4cc(C#N)cc(-n5c6ccccc6c6ccccc65)c4)ccc32)c1 ; N#Cc1cc(C#N)cc(-c2cc(C#N)ccc2-n2c3ccccc3c3cc(-c4cc(-n5c6ccccc6c6ccccc65)cc(C(F)(F)F)c4)ccc32)c1 ; N#Cc1cc(C#N)cc(-c2cc(C#N)ccc2-n2c3ccccc3c3cc(-c4cc(-c5ccccc5)cc(-n5c6ccccc6c6ccccc65)c4)ccc32)c1]

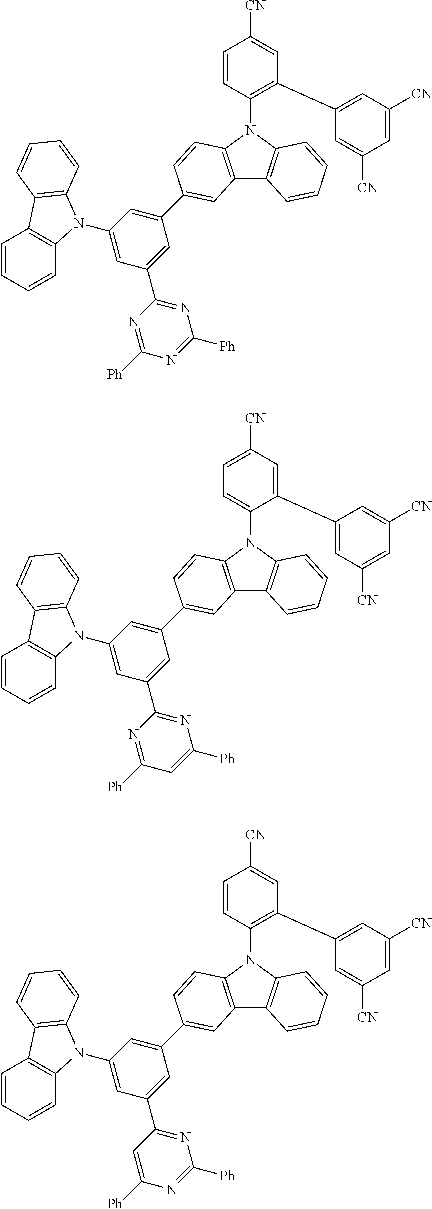 [SMILES: N#Cc1cc(C#N)cc(-c2cc(C#N)ccc2-n2c3ccccc3c3cc(-c4cc(-c5nc(-c6ccccc6)nc(-c6ccccc6)n5)cc(-n5c6ccccc6c6ccccc65)c4)ccc32)c1 ; N#Cc1cc(C#N)cc(-c2cc(C#N)ccc2-n2c3ccccc3c3cc(-c4cc(-c5nc(-c6ccccc6)cc(-c6ccccc6)n5)cc(-n5c6ccccc6c6ccccc65)c4)ccc32)c1 ; N#Cc1cc(C#N)cc(-c2cc(C#N)ccc2-n2c3ccccc3c3cc(-c4cc(-c5cc(-c6ccccc6)nc(-c6ccccc6)n5)cc(-n5c6ccccc6c6ccccc65)c4)ccc32)c1]

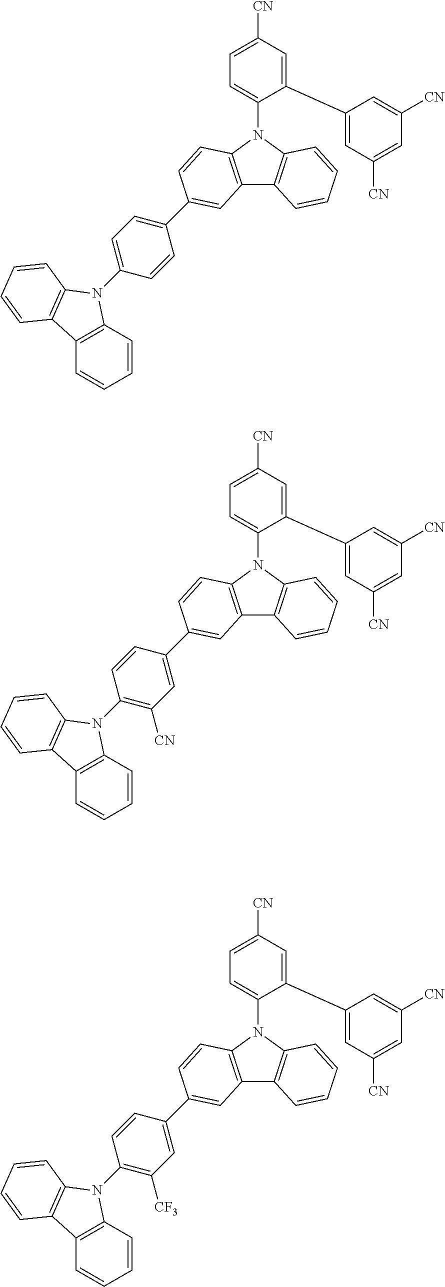 [SMILES: N#Cc1cc(C#N)cc(-c2cc(C#N)ccc2-n2c3ccccc3c3cc(-c4ccc(-n5c6ccccc6c6ccccc65)cc4)ccc32)c1 ; N#Cc1cc(C#N)cc(-c2cc(C#N)ccc2-n2c3ccccc3c3cc(-c4ccc(-n5c6ccccc6c6ccccc65)c(C#N)c4)ccc32)c1 ; N#Cc1cc(C#N)cc(-c2cc(C#N)ccc2-n2c3ccccc3c3cc(-c4ccc(-n5c6ccccc6c6ccccc65)c(C(F)(F)F)c4)ccc32)c1]

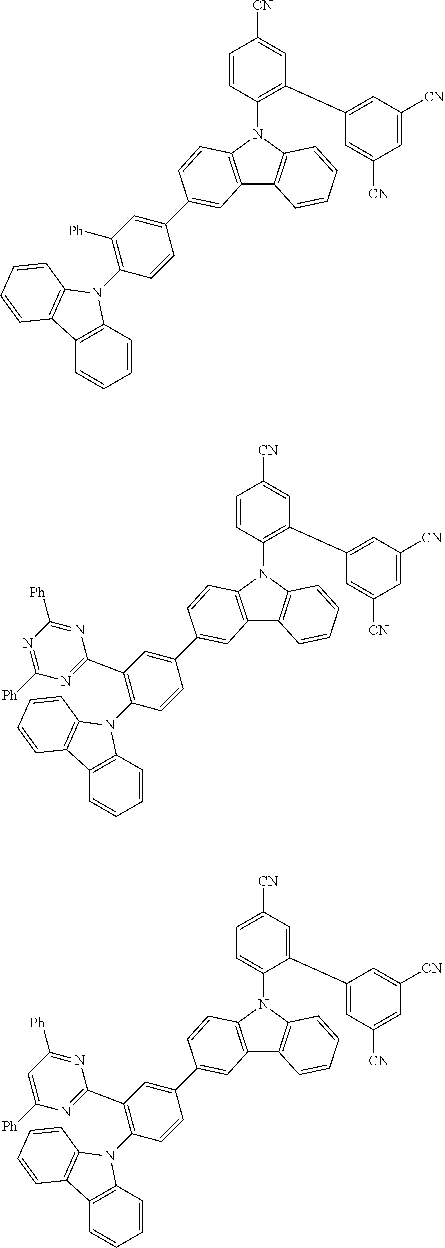 [SMILES: N#Cc1cc(C#N)cc(-c2cc(C#N)ccc2-n2c3ccccc3c3cc(-c4ccc(-n5c6ccccc6c6ccccc65)c(-c5ccccc5)c4)ccc32)c1 ; N#Cc1cc(C#N)cc(-c2cc(C#N)ccc2-n2c3ccccc3c3cc(-c4ccc(-n5c6ccccc6c6ccccc65)c(-c5nc(-c6ccccc6)nc(-c6ccccc6)n5)c4)ccc32)c1 ; N#Cc1cc(C#N)cc(-c2cc(C#N)ccc2-n2c3ccccc3c3cc(-c4ccc(-n5c6ccccc6c6ccccc65)c(-c5nc(-c6ccccc6)cc(-c6ccccc6)n5)c4)ccc32)c1]

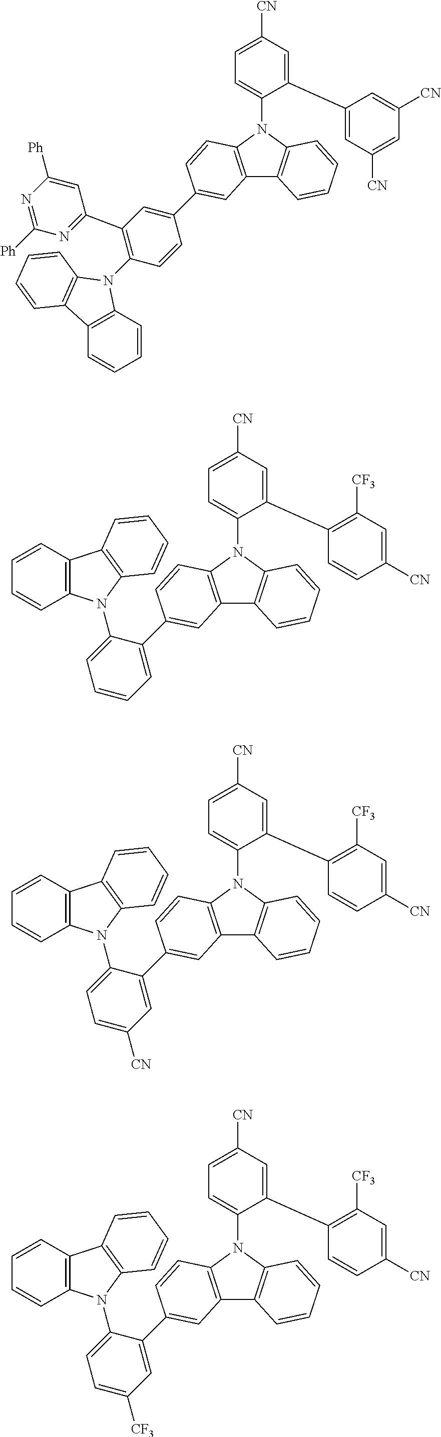 [SMILES: N#Cc1cc(C#N)cc(-c2cc(C#N)ccc2-n2c3ccccc3c3cc(-c4ccc(-n5c6ccccc6c6ccccc65)c(-c5cc(-c6ccccc6)nc(-c6ccccc6)n5)c4)ccc32)c1 ; N#Cc1ccc(-n2c3ccccc3c3cc(-c4ccccc4-n4c5ccccc5c5ccccc54)ccc32)c(-c2ccc(C#N)cc2C(F)(F)F)c1 ; N#Cc1ccc(-n2c3ccccc3c3ccccc32)c(-c2ccc3c(c2)c2ccccc2n3-c2ccc(C#N)cc2-c2ccc(C#N)cc2C(F)(F)F)c1 ; N#Cc1ccc(-n2c3ccccc3c3cc(-c4cc(C(F)(F)F)ccc4-n4c5ccccc5c5ccccc54)ccc32)c(-c2ccc(C#N)cc2C(F)(F)F)c1]

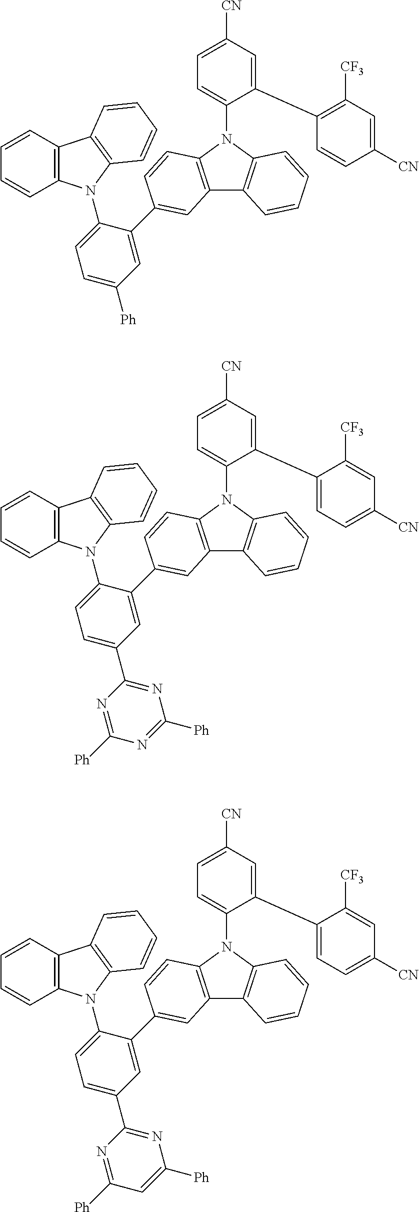 [SMILES: N#Cc1ccc(-n2c3ccccc3c3cc(-c4cc(-c5ccccc5)ccc4-n4c5ccccc5c5ccccc54)ccc32)c(-c2ccc(C#N)cc2C(F)(F)F)c1 ; N#Cc1ccc(-n2c3ccccc3c3cc(-c4cc(-c5nc(-c6ccccc6)nc(-c6ccccc6)n5)ccc4-n4c5ccccc5c5ccccc54)ccc32)c(-c2ccc(C#N)cc2C(F)(F)F)c1 ; N#Cc1ccc(-n2c3ccccc3c3cc(-c4cc(-c5nc(-c6ccccc6)cc(-c6ccccc6)n5)ccc4-n4c5ccccc5c5ccccc54)ccc32)c(-c2ccc(C#N)cc2C(F)(F)F)c1]

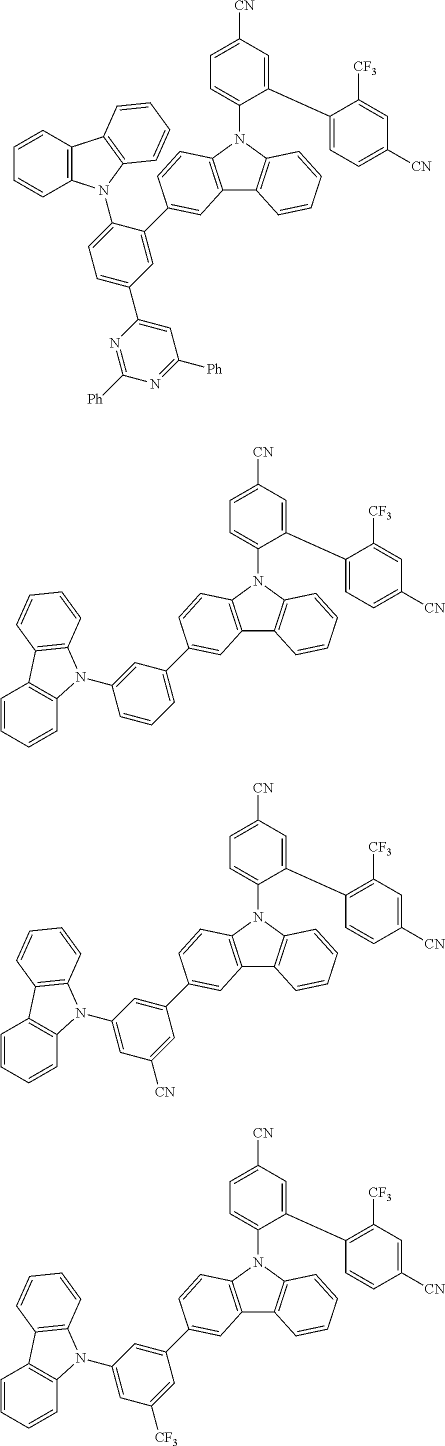 [SMILES: N#Cc1ccc(-n2c3ccccc3c3cc(-c4cc(-c5cc(-c6ccccc6)nc(-c6ccccc6)n5)ccc4-n4c5ccccc5c5ccccc54)ccc32)c(-c2ccc(C#N)cc2C(F)(F)F)c1 ; N#Cc1ccc(-n2c3ccccc3c3cc(-c4cccc(-n5c6ccccc6c6ccccc65)c4)ccc32)c(-c2ccc(C#N)cc2C(F)(F)F)c1 ; N#Cc1cc(-c2ccc3c(c2)c2ccccc2n3-c2ccc(C#N)cc2-c2ccc(C#N)cc2C(F)(F)F)cc(-n2c3ccccc3c3ccccc32)c1 ; N#Cc1ccc(-n2c3ccccc3c3cc(-c4cc(-n5c6ccccc6c6ccccc65)cc(C(F)(F)F)c4)ccc32)c(-c2ccc(C#N)cc2C(F)(F)F)c1]

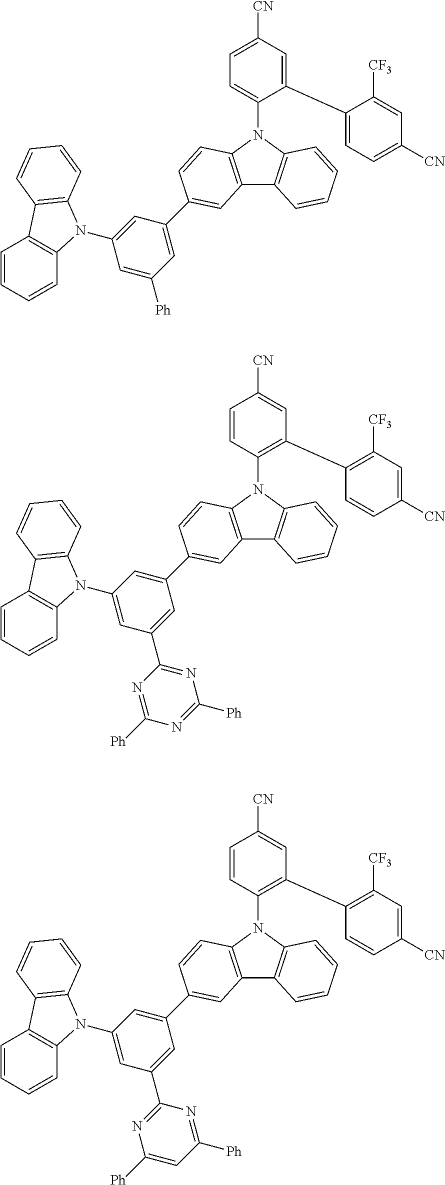 [SMILES: N#Cc1ccc(-n2c3ccccc3c3cc(-c4cc(-c5ccccc5)cc(-n5c6ccccc6c6ccccc65)c4)ccc32)c(-c2ccc(C#N)cc2C(F)(F)F)c1 ; N#Cc1ccc(-n2c3ccccc3c3cc(-c4cc(-c5nc(-c6ccccc6)nc(-c6ccccc6)n5)cc(-n5c6ccccc6c6ccccc65)c4)ccc32)c(-c2ccc(C#N)cc2C(F)(F)F)c1 ; N#Cc1ccc(-n2c3ccccc3c3cc(-c4cc(-c5nc(-c6ccccc6)cc(-c6ccccc6)n5)cc(-n5c6ccccc6c6ccccc65)c4)ccc32)c(-c2ccc(C#N)cc2C(F)(F)F)c1]

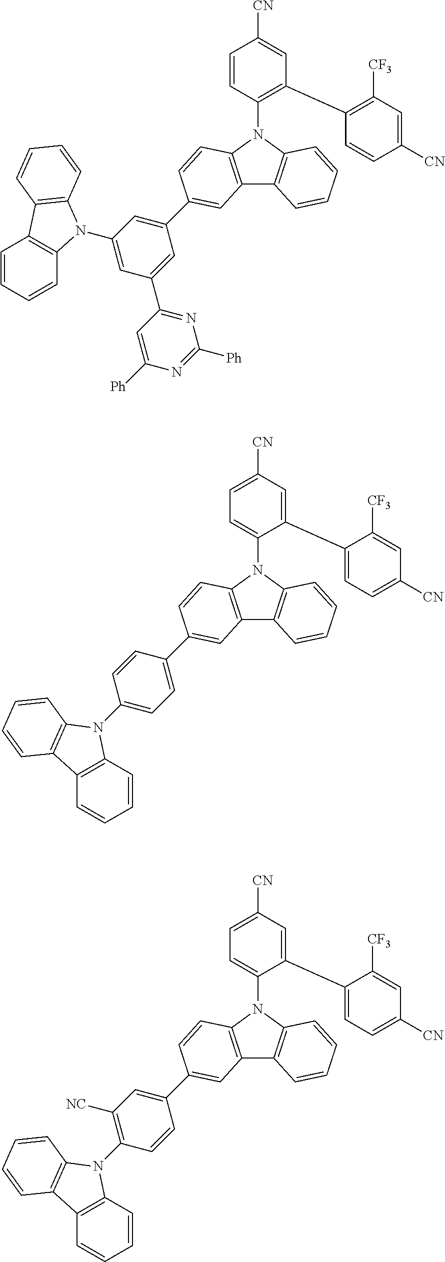 [SMILES: N#Cc1ccc(-n2c3ccccc3c3cc(-c4cc(-c5cc(-c6ccccc6)nc(-c6ccccc6)n5)cc(-n5c6ccccc6c6ccccc65)c4)ccc32)c(-c2ccc(C#N)cc2C(F)(F)F)c1 ; N#Cc1ccc(-n2c3ccccc3c3cc(-c4ccc(-n5c6ccccc6c6ccccc65)cc4)ccc32)c(-c2ccc(C#N)cc2C(F)(F)F)c1 ; N#Cc1ccc(-n2c3ccccc3c3cc(-c4ccc(-n5c6ccccc6c6ccccc65)c(C#N)c4)ccc32)c(-c2ccc(C#N)cc2C(F)(F)F)c1]

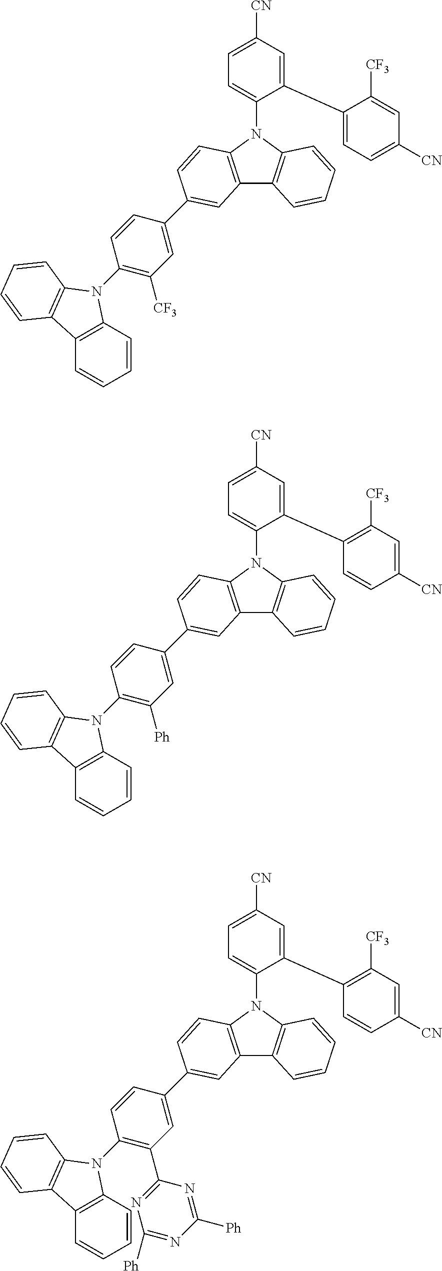 [SMILES: N#Cc1ccc(-n2c3ccccc3c3cc(-c4ccc(-n5c6ccccc6c6ccccc65)c(C(F)(F)F)c4)ccc32)c(-c2ccc(C#N)cc2C(F)(F)F)c1 ; N#Cc1ccc(-n2c3ccccc3c3cc(-c4ccc(-n5c6ccccc6c6ccccc65)c(-c5ccccc5)c4)ccc32)c(-c2ccc(C#N)cc2C(F)(F)F)c1 ; N#Cc1ccc(-n2c3ccccc3c3cc(-c4ccc(-n5c6ccccc6c6ccccc65)c(-c5nc(-c6ccccc6)nc(-c6ccccc6)n5)c4)ccc32)c(-c2ccc(C#N)cc2C(F)(F)F)c1]

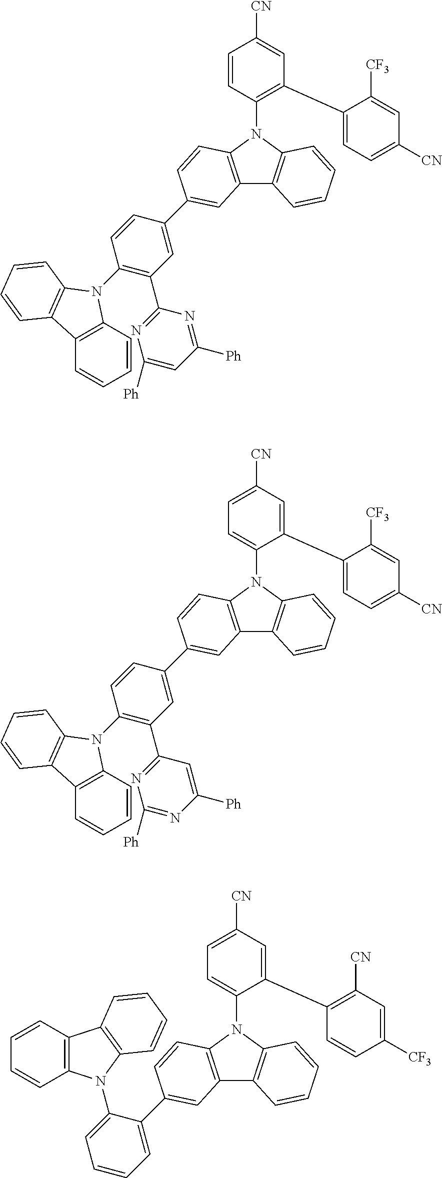 [SMILES: N#Cc1ccc(-n2c3ccccc3c3cc(-c4ccc(-n5c6ccccc6c6ccccc65)c(-c5nc(-c6ccccc6)cc(-c6ccccc6)n5)c4)ccc32)c(-c2ccc(C#N)cc2C(F)(F)F)c1 ; N#Cc1ccc(-n2c3ccccc3c3cc(-c4ccc(-n5c6ccccc6c6ccccc65)c(-c5cc(-c6ccccc6)nc(-c6ccccc6)n5)c4)ccc32)c(-c2ccc(C#N)cc2C(F)(F)F)c1 ; N#Cc1ccc(-n2c3ccccc3c3cc(-c4ccccc4-n4c5ccccc5c5ccccc54)ccc32)c(-c2ccc(C(F)(F)F)cc2C#N)c1]

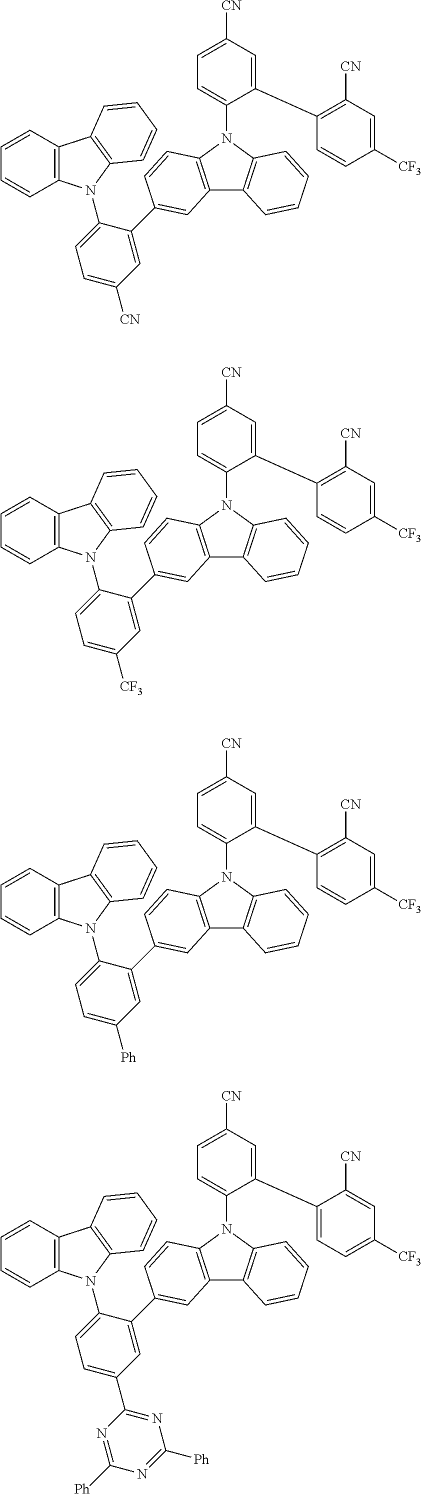 [SMILES: N#Cc1ccc(-n2c3ccccc3c3ccccc32)c(-c2ccc3c(c2)c2ccccc2n3-c2ccc(C#N)cc2-c2ccc(C(F)(F)F)cc2C#N)c1 ; N#Cc1ccc(-n2c3ccccc3c3cc(-c4cc(C(F)(F)F)ccc4-n4c5ccccc5c5ccccc54)ccc32)c(-c2ccc(C(F)(F)F)cc2C#N)c1 ; N#Cc1ccc(-n2c3ccccc3c3cc(-c4cc(-c5ccccc5)ccc4-n4c5ccccc5c5ccccc54)ccc32)c(-c2ccc(C(F)(F)F)cc2C#N)c1 ; N#Cc1ccc(-n2c3ccccc3c3cc(-c4cc(-c5nc(-c6ccccc6)nc(-c6ccccc6)n5)ccc4-n4c5ccccc5c5ccccc54)ccc32)c(-c2ccc(C(F)(F)F)cc2C#N)c1]

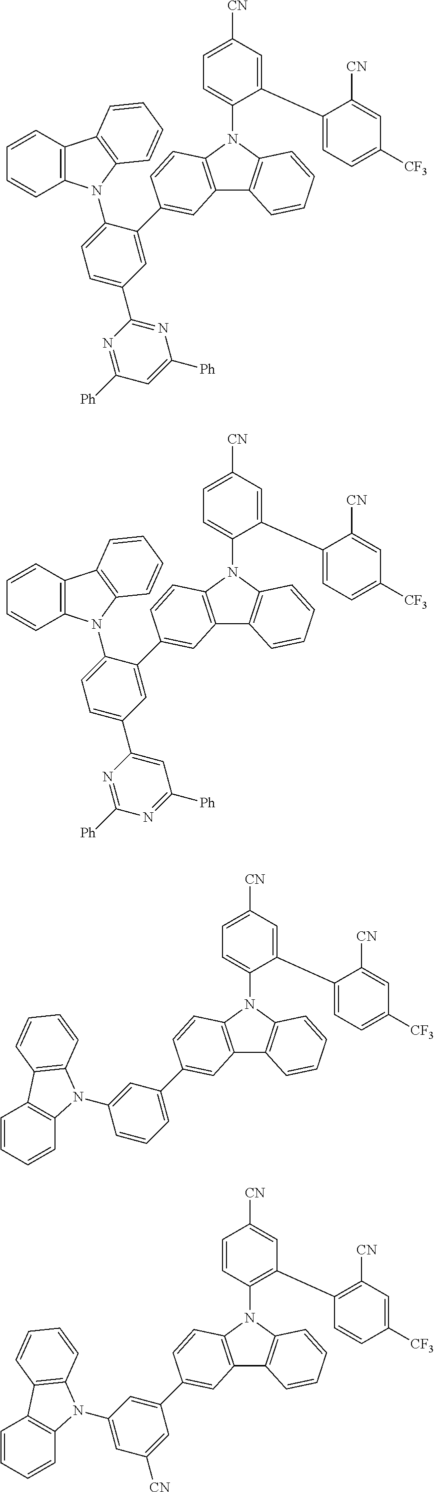 [SMILES: N#Cc1ccc(-n2c3ccccc3c3cc(-c4cc(-c5nc(-c6ccccc6)cc(-c6ccccc6)n5)ccc4-n4c5ccccc5c5ccccc54)ccc32)c(-c2ccc(C(F)(F)F)cc2C#N)c1 ; N#Cc1ccc(-n2c3ccccc3c3cc(-c4cc(-c5cc(-c6ccccc6)nc(-c6ccccc6)n5)ccc4-n4c5ccccc5c5ccccc54)ccc32)c(-c2ccc(C(F)(F)F)cc2C#N)c1 ; N#Cc1ccc(-n2c3ccccc3c3cc(-c4cccc(-n5c6ccccc6c6ccccc65)c4)ccc32)c(-c2ccc(C(F)(F)F)cc2C#N)c1 ; N#Cc1cc(-c2ccc3c(c2)c2ccccc2n3-c2ccc(C#N)cc2-c2ccc(C(F)(F)F)cc2C#N)cc(-n2c3ccccc3c3ccccc32)c1]

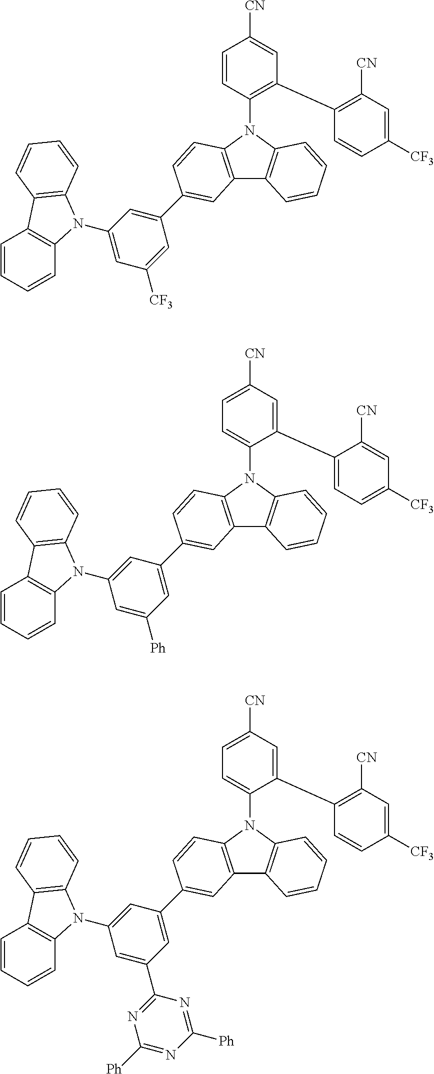 [SMILES: N#Cc1ccc(-n2c3ccccc3c3cc(-c4cc(-n5c6ccccc6c6ccccc65)cc(C(F)(F)F)c4)ccc32)c(-c2ccc(C(F)(F)F)cc2C#N)c1 ; N#Cc1ccc(-n2c3ccccc3c3cc(-c4cc(-c5ccccc5)cc(-n5c6ccccc6c6ccccc65)c4)ccc32)c(-c2ccc(C(F)(F)F)cc2C#N)c1 ; N#Cc1ccc(-n2c3ccccc3c3cc(-c4cc(-c5nc(-c6ccccc6)nc(-c6ccccc6)n5)cc(-n5c6ccccc6c6ccccc65)c4)ccc32)c(-c2ccc(C(F)(F)F)cc2C#N)c1]

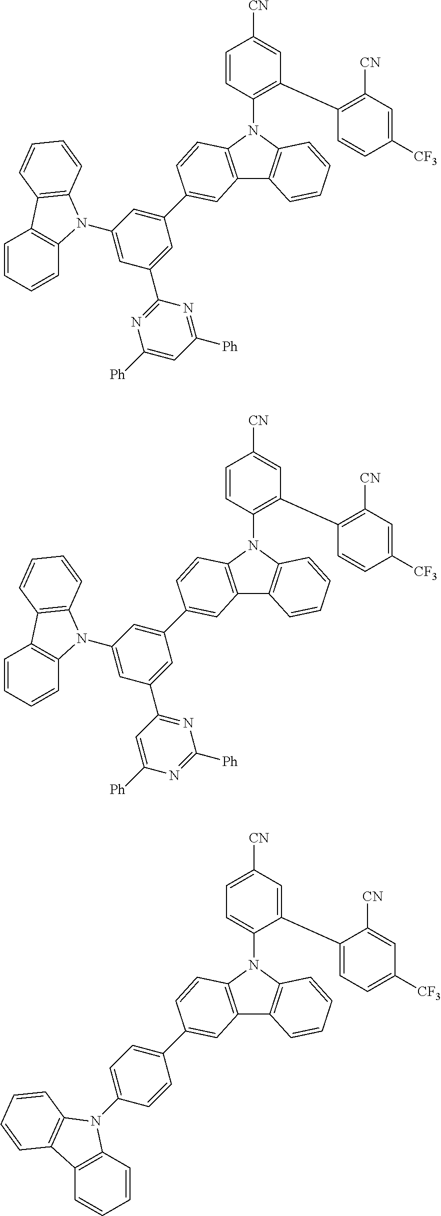 [SMILES: N#Cc1ccc(-n2c3ccccc3c3cc(-c4cc(-c5nc(-c6ccccc6)cc(-c6ccccc6)n5)cc(-n5c6ccccc6c6ccccc65)c4)ccc32)c(-c2ccc(C(F)(F)F)cc2C#N)c1 ; N#Cc1ccc(-n2c3ccccc3c3cc(-c4cc(-c5cc(-c6ccccc6)nc(-c6ccccc6)n5)cc(-n5c6ccccc6c6ccccc65)c4)ccc32)c(-c2ccc(C(F)(F)F)cc2C#N)c1 ; N#Cc1ccc(-n2c3ccccc3c3cc(-c4ccc(-n5c6ccccc6c6ccccc65)cc4)ccc32)c(-c2ccc(C(F)(F)F)cc2C#N)c1]

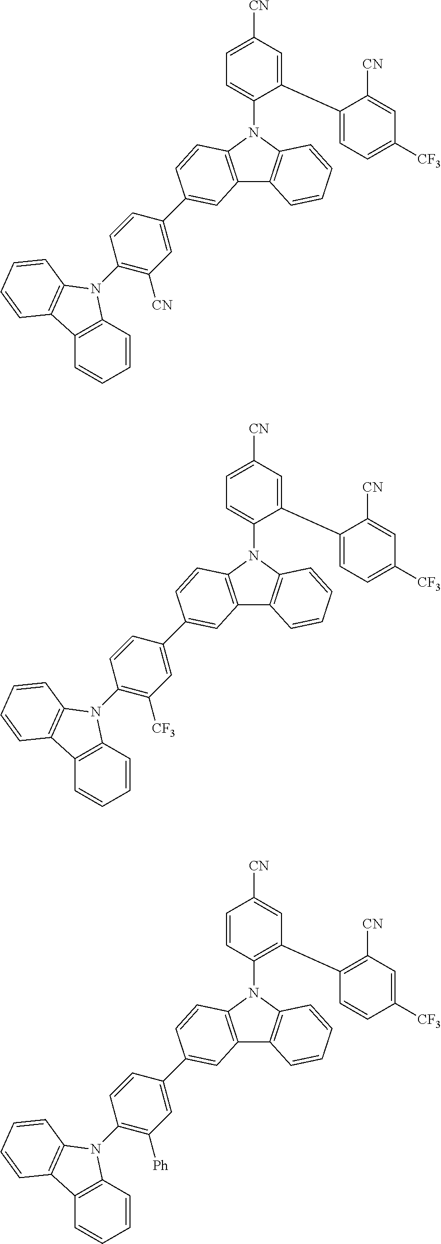 [SMILES: N#Cc1ccc(-n2c3ccccc3c3cc(-c4ccc(-n5c6ccccc6c6ccccc65)c(C#N)c4)ccc32)c(-c2ccc(C(F)(F)F)cc2C#N)c1 ; N#Cc1ccc(-n2c3ccccc3c3cc(-c4ccc(-n5c6ccccc6c6ccccc65)c(C(F)(F)F)c4)ccc32)c(-c2ccc(C(F)(F)F)cc2C#N)c1 ; N#Cc1ccc(-n2c3ccccc3c3cc(-c4ccc(-n5c6ccccc6c6ccccc65)c(-c5ccccc5)c4)ccc32)c(-c2ccc(C(F)(F)F)cc2C#N)c1]

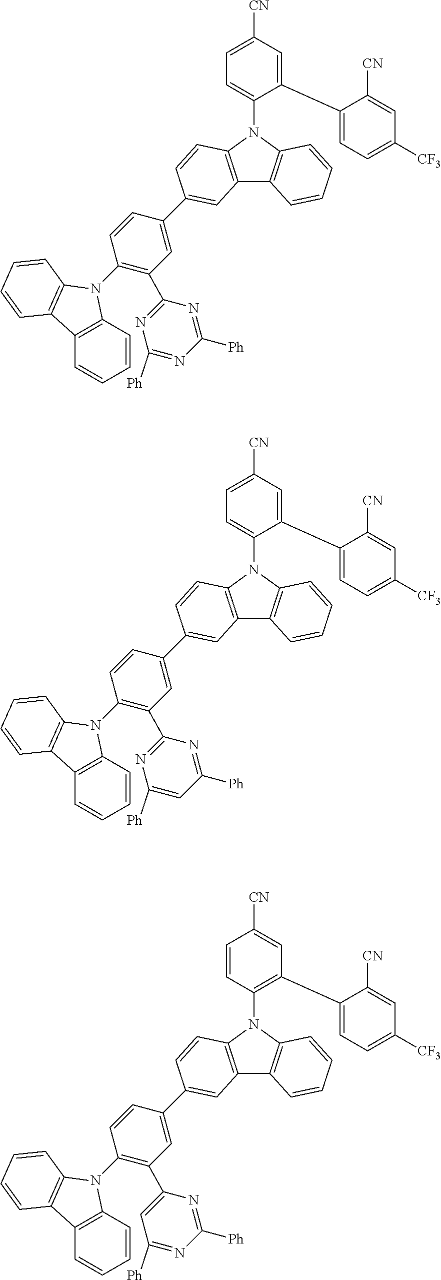 [SMILES: N#Cc1ccc(-n2c3ccccc3c3cc(-c4ccc(-n5c6ccccc6c6ccccc65)c(-c5nc(-c6ccccc6)nc(-c6ccccc6)n5)c4)ccc32)c(-c2ccc(C(F)(F)F)cc2C#N)c1 ; N#Cc1ccc(-n2c3ccccc3c3cc(-c4ccc(-n5c6ccccc6c6ccccc65)c(-c5nc(-c6ccccc6)cc(-c6ccccc6)n5)c4)ccc32)c(-c2ccc(C(F)(F)F)cc2C#N)c1 ; N#Cc1ccc(-n2c3ccccc3c3cc(-c4ccc(-n5c6ccccc6c6ccccc65)c(-c5cc(-c6ccccc6)nc(-c6ccccc6)n5)c4)ccc32)c(-c2ccc(C(F)(F)F)cc2C#N)c1]

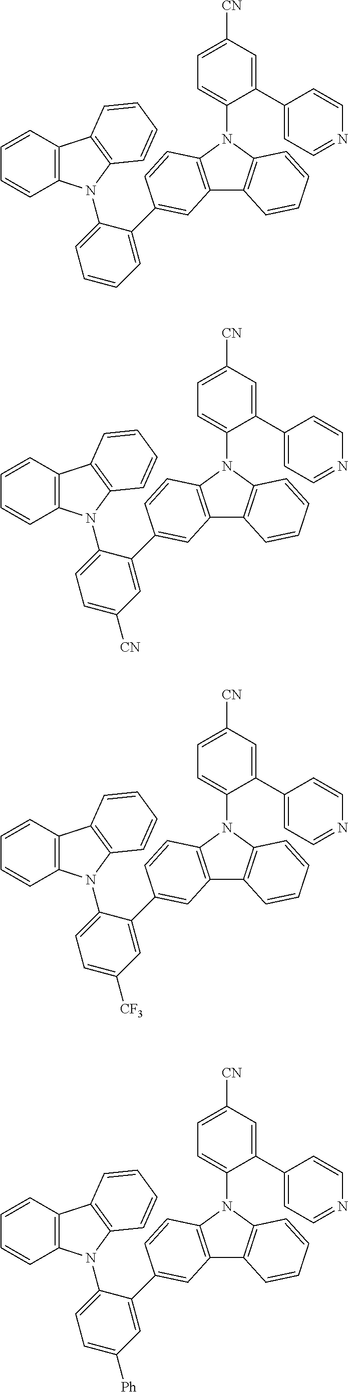 [SMILES: N#Cc1ccc(-n2c3ccccc3c3cc(-c4ccccc4-n4c5ccccc5c5ccccc54)ccc32)c(-c2ccncc2)c1 ; N#Cc1ccc(-n2c3ccccc3c3ccccc32)c(-c2ccc3c(c2)c2ccccc2n3-c2ccc(C#N)cc2-c2ccncc2)c1 ; N#Cc1ccc(-n2c3ccccc3c3cc(-c4cc(C(F)(F)F)ccc4-n4c5ccccc5c5ccccc54)ccc32)c(-c2ccncc2)c1 ; N#Cc1ccc(-n2c3ccccc3c3cc(-c4cc(-c5ccccc5)ccc4-n4c5ccccc5c5ccccc54)ccc32)c(-c2ccncc2)c1]

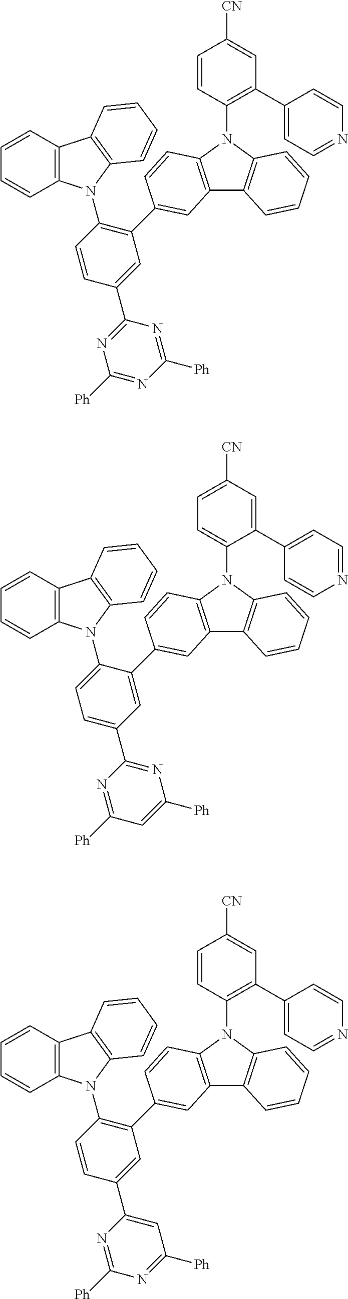 [SMILES: N#Cc1ccc(-n2c3ccccc3c3cc(-c4cc(-c5nc(-c6ccccc6)nc(-c6ccccc6)n5)ccc4-n4c5ccccc5c5ccccc54)ccc32)c(-c2ccncc2)c1 ; N#Cc1ccc(-n2c3ccccc3c3cc(-c4cc(-c5nc(-c6ccccc6)cc(-c6ccccc6)n5)ccc4-n4c5ccccc5c5ccccc54)ccc32)c(-c2ccncc2)c1 ; N#Cc1ccc(-n2c3ccccc3c3cc(-c4cc(-c5cc(-c6ccccc6)nc(-c6ccccc6)n5)ccc4-n4c5ccccc5c5ccccc54)ccc32)c(-c2ccncc2)c1]

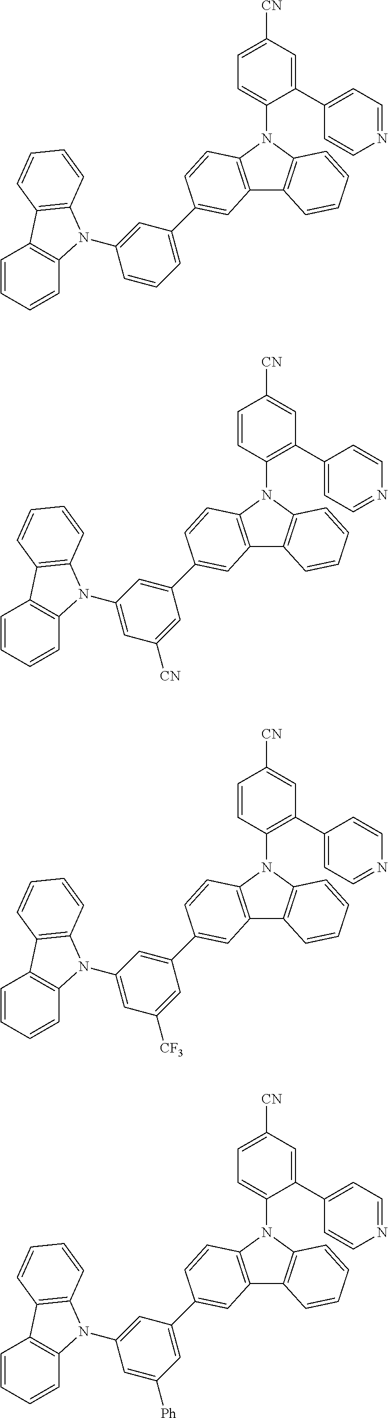 [SMILES: N#Cc1ccc(-n2c3ccccc3c3cc(-c4cccc(-n5c6ccccc6c6ccccc65)c4)ccc32)c(-c2ccncc2)c1 ; N#Cc1cc(-c2ccc3c(c2)c2ccccc2n3-c2ccc(C#N)cc2-c2ccncc2)cc(-n2c3ccccc3c3ccccc32)c1 ; N#Cc1ccc(-n2c3ccccc3c3cc(-c4cc(-n5c6ccccc6c6ccccc65)cc(C(F)(F)F)c4)ccc32)c(-c2ccncc2)c1 ; N#Cc1ccc(-n2c3ccccc3c3cc(-c4cc(-c5ccccc5)cc(-n5c6ccccc6c6ccccc65)c4)ccc32)c(-c2ccncc2)c1]

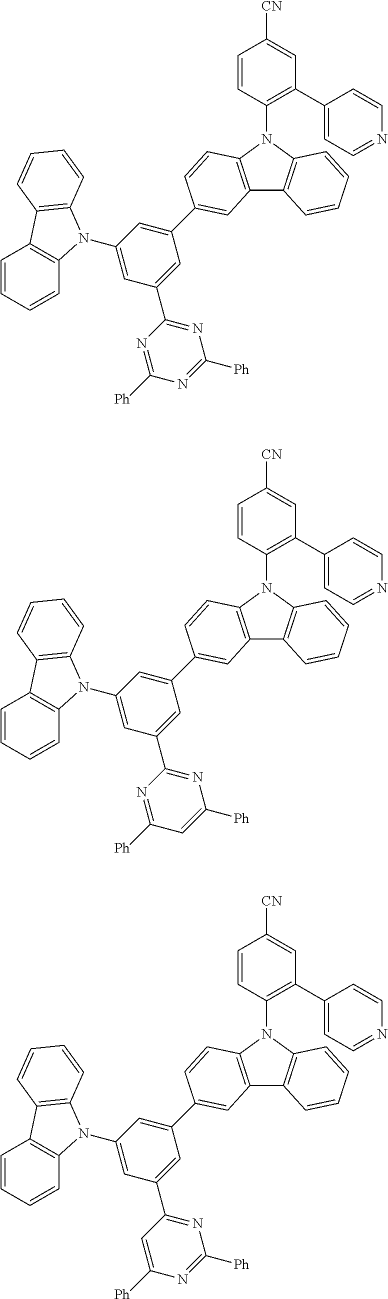 [SMILES: N#Cc1ccc(-n2c3ccccc3c3cc(-c4cc(-c5nc(-c6ccccc6)nc(-c6ccccc6)n5)cc(-n5c6ccccc6c6ccccc65)c4)ccc32)c(-c2ccncc2)c1 ; N#Cc1ccc(-n2c3ccccc3c3cc(-c4cc(-c5nc(-c6ccccc6)cc(-c6ccccc6)n5)cc(-n5c6ccccc6c6ccccc65)c4)ccc32)c(-c2ccncc2)c1 ; N#Cc1ccc(-n2c3ccccc3c3cc(-c4cc(-c5cc(-c6ccccc6)nc(-c6ccccc6)n5)cc(-n5c6ccccc6c6ccccc65)c4)ccc32)c(-c2ccncc2)c1]

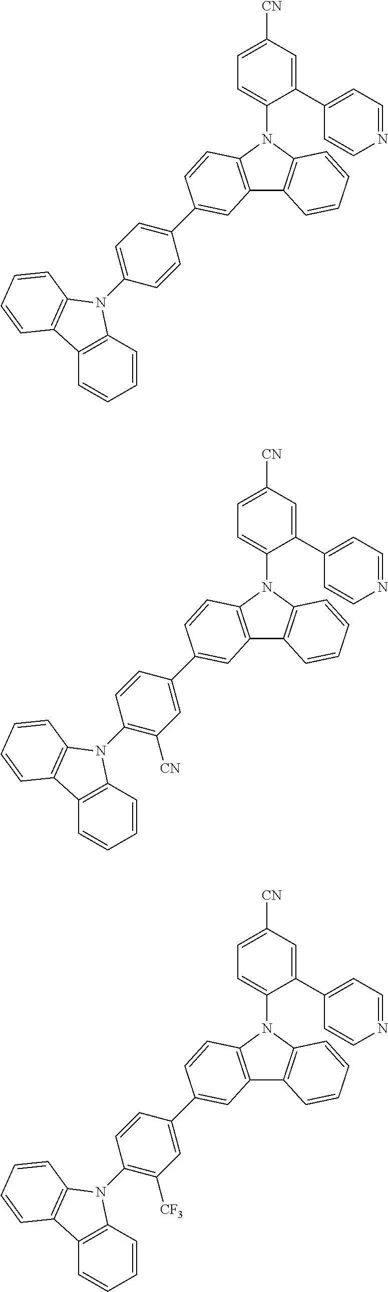 [SMILES: N#Cc1ccc(-n2c3ccccc3c3cc(-c4ccc(-n5c6ccccc6c6ccccc65)cc4)ccc32)c(-c2ccncc2)c1 ; N#Cc1ccc(-n2c3ccccc3c3cc(-c4ccc(-n5c6ccccc6c6ccccc65)c(C#N)c4)ccc32)c(-c2ccncc2)c1 ; N#Cc1ccc(-n2c3ccccc3c3cc(-c4ccc(-n5c6ccccc6c6ccccc65)c(C(F)(F)F)c4)ccc32)c(-c2ccncc2)c1]

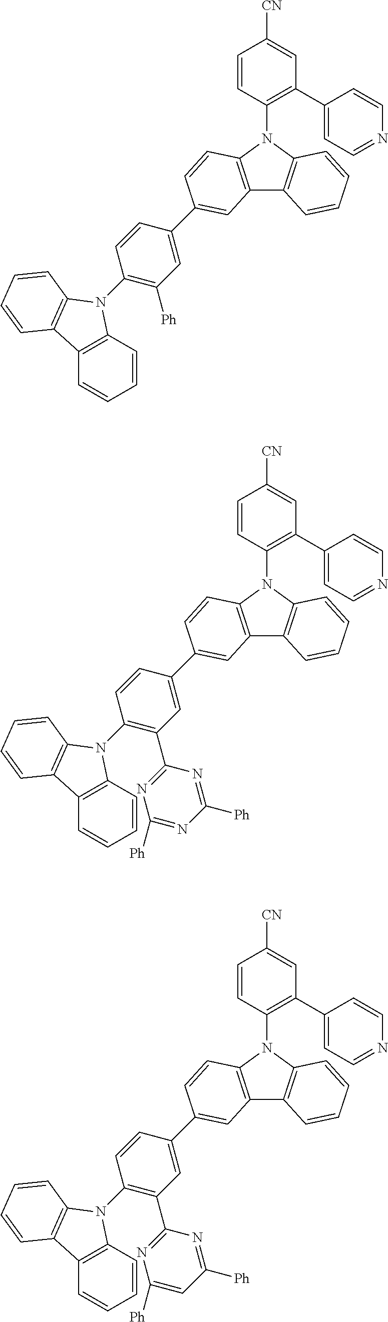 [SMILES: N#Cc1ccc(-n2c3ccccc3c3cc(-c4ccc(-n5c6ccccc6c6ccccc65)c(-c5ccccc5)c4)ccc32)c(-c2ccncc2)c1 ; N#Cc1ccc(-n2c3ccccc3c3cc(-c4ccc(-n5c6ccccc6c6ccccc65)c(-c5nc(-c6ccccc6)nc(-c6ccccc6)n5)c4)ccc32)c(-c2ccncc2)c1 ; N#Cc1ccc(-n2c3ccccc3c3cc(-c4ccc(-n5c6ccccc6c6ccccc65)c(-c5nc(-c6ccccc6)cc(-c6ccccc6)n5)c4)ccc32)c(-c2ccncc2)c1]

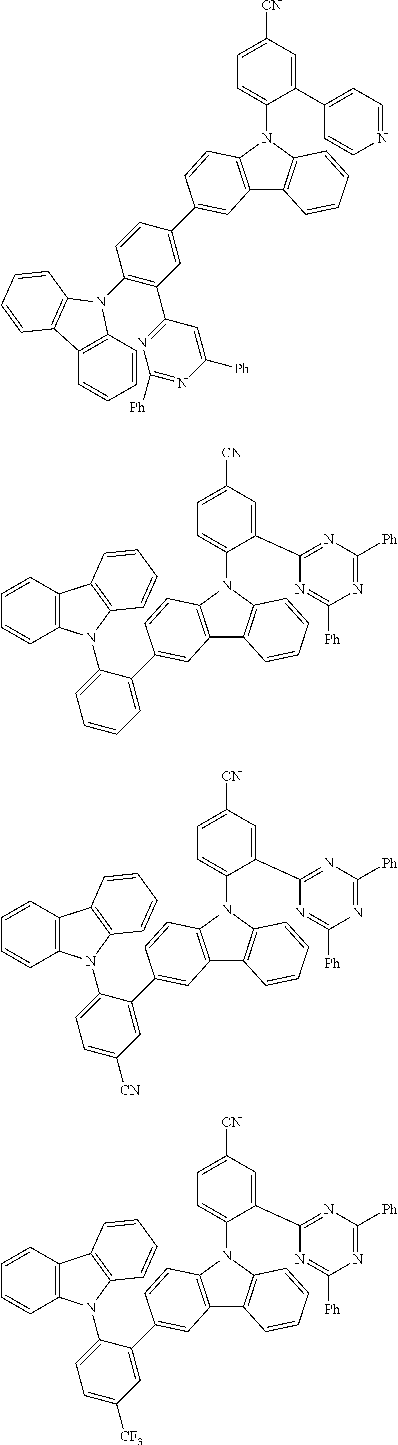 [SMILES: N#Cc1ccc(-n2c3ccccc3c3cc(-c4ccc(-n5c6ccccc6c6ccccc65)c(-c5cc(-c6ccccc6)nc(-c6ccccc6)n5)c4)ccc32)c(-c2ccncc2)c1 ; N#Cc1ccc(-n2c3ccccc3c3cc(-c4ccccc4-n4c5ccccc5c5ccccc54)ccc32)c(-c2nc(-c3ccccc3)nc(-c3ccccc3)n2)c1 ; N#Cc1ccc(-n2c3ccccc3c3ccccc32)c(-c2ccc3c(c2)c2ccccc2n3-c2ccc(C#N)cc2-c2nc(-c3ccccc3)nc(-c3ccccc3)n2)c1 ; N#Cc1ccc(-n2c3ccccc3c3cc(-c4cc(C(F)(F)F)ccc4-n4c5ccccc5c5ccccc54)ccc32)c(-c2nc(-c3ccccc3)nc(-c3ccccc3)n2)c1]

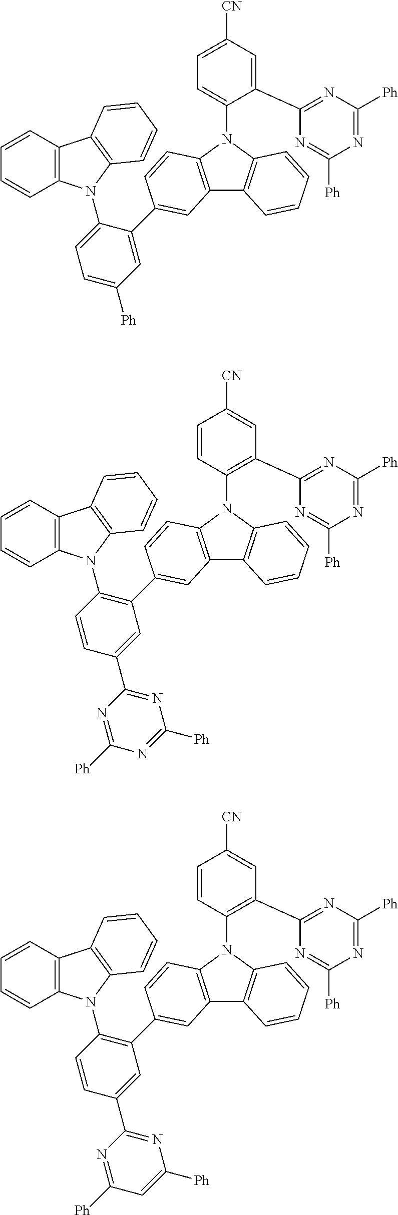 [SMILES: N#Cc1ccc(-n2c3ccccc3c3cc(-c4cc(-c5ccccc5)ccc4-n4c5ccccc5c5ccccc54)ccc32)c(-c2nc(-c3ccccc3)nc(-c3ccccc3)n2)c1 ; N#Cc1ccc(-n2c3ccccc3c3cc(-c4cc(-c5nc(-c6ccccc6)nc(-c6ccccc6)n5)ccc4-n4c5ccccc5c5ccccc54)ccc32)c(-c2nc(-c3ccccc3)nc(-c3ccccc3)n2)c1 ; N#Cc1ccc(-n2c3ccccc3c3cc(-c4cc(-c5nc(-c6ccccc6)cc(-c6ccccc6)n5)ccc4-n4c5ccccc5c5ccccc54)ccc32)c(-c2nc(-c3ccccc3)nc(-c3ccccc3)n2)c1]

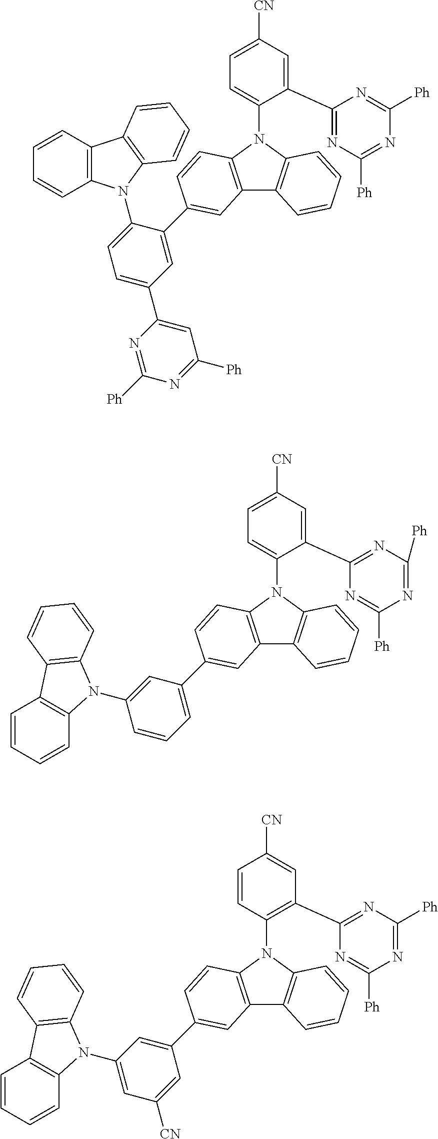 [SMILES: N#Cc1ccc(-n2c3ccccc3c3cc(-c4cc(-c5cc(-c6ccccc6)nc(-c6ccccc6)n5)ccc4-n4c5ccccc5c5ccccc54)ccc32)c(-c2nc(-c3ccccc3)nc(-c3ccccc3)n2)c1 ; N#Cc1ccc(-n2c3ccccc3c3cc(-c4cccc(-n5c6ccccc6c6ccccc65)c4)ccc32)c(-c2nc(-c3ccccc3)nc(-c3ccccc3)n2)c1 ; N#Cc1cc(-c2ccc3c(c2)c2ccccc2n3-c2ccc(C#N)cc2-c2nc(-c3ccccc3)nc(-c3ccccc3)n2)cc(-n2c3ccccc3c3ccccc32)c1]

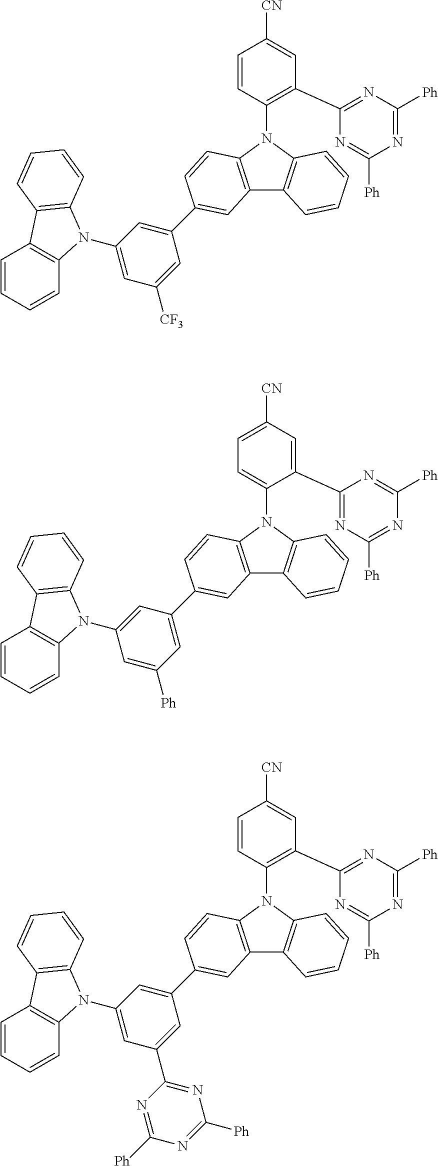 [SMILES: N#Cc1ccc(-n2c3ccccc3c3cc(-c4cc(-n5c6ccccc6c6ccccc65)cc(C(F)(F)F)c4)ccc32)c(-c2nc(-c3ccccc3)nc(-c3ccccc3)n2)c1 ; N#Cc1ccc(-n2c3ccccc3c3cc(-c4cc(-c5ccccc5)cc(-n5c6ccccc6c6ccccc65)c4)ccc32)c(-c2nc(-c3ccccc3)nc(-c3ccccc3)n2)c1 ; N#Cc1ccc(-n2c3ccccc3c3cc(-c4cc(-c5nc(-c6ccccc6)nc(-c6ccccc6)n5)cc(-n5c6ccccc6c6ccccc65)c4)ccc32)c(-c2nc(-c3ccccc3)nc(-c3ccccc3)n2)c1]

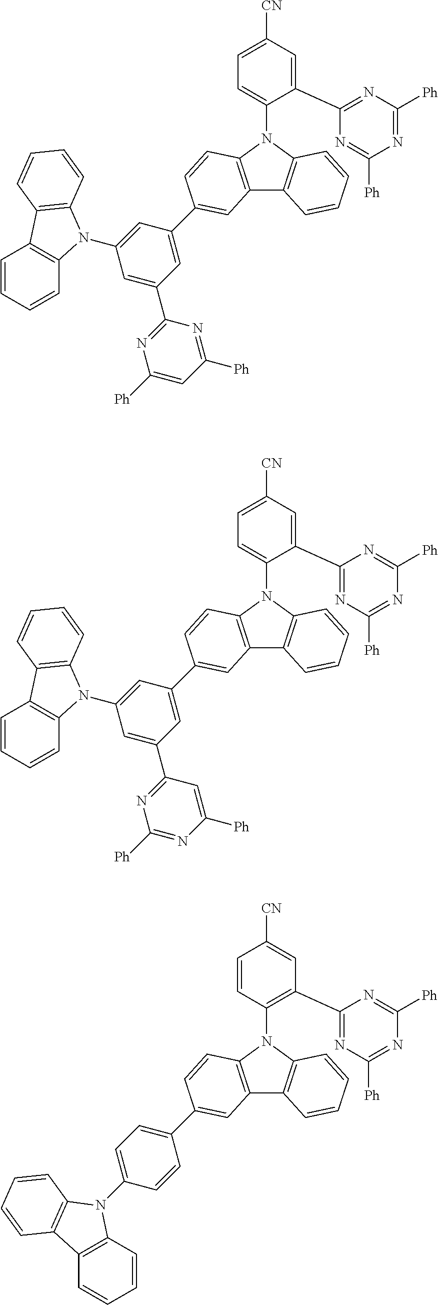 [SMILES: N#Cc1ccc(-n2c3ccccc3c3cc(-c4cc(-c5nc(-c6ccccc6)cc(-c6ccccc6)n5)cc(-n5c6ccccc6c6ccccc65)c4)ccc32)c(-c2nc(-c3ccccc3)nc(-c3ccccc3)n2)c1 ; N#Cc1ccc(-n2c3ccccc3c3cc(-c4cc(-c5cc(-c6ccccc6)nc(-c6ccccc6)n5)cc(-n5c6ccccc6c6ccccc65)c4)ccc32)c(-c2nc(-c3ccccc3)nc(-c3ccccc3)n2)c1 ; N#Cc1ccc(-n2c3ccccc3c3cc(-c4ccc(-n5c6ccccc6c6ccccc65)cc4)ccc32)c(-c2nc(-c3ccccc3)nc(-c3ccccc3)n2)c1]

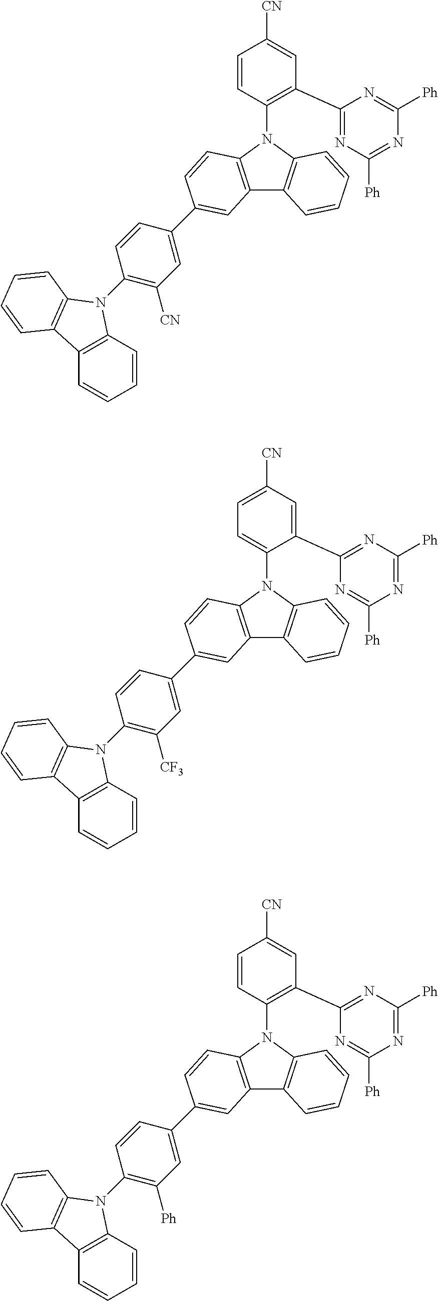 [SMILES: N#Cc1ccc(-n2c3ccccc3c3cc(-c4ccc(-n5c6ccccc6c6ccccc65)c(C#N)c4)ccc32)c(-c2nc(-c3ccccc3)nc(-c3ccccc3)n2)c1 ; N#Cc1ccc(-n2c3ccccc3c3cc(-c4ccc(-n5c6ccccc6c6ccccc65)c(C(F)(F)F)c4)ccc32)c(-c2nc(-c3ccccc3)nc(-c3ccccc3)n2)c1 ; N#Cc1ccc(-n2c3ccccc3c3cc(-c4ccc(-n5c6ccccc6c6ccccc65)c(-c5ccccc5)c4)ccc32)c(-c2nc(-c3ccccc3)nc(-c3ccccc3)n2)c1]

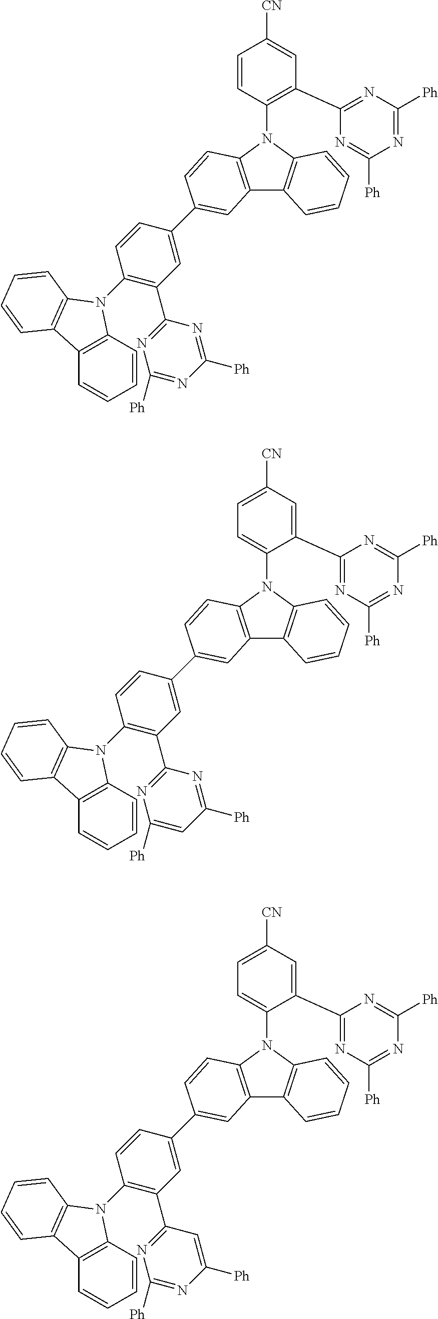 [SMILES: N#Cc1ccc(-n2c3ccccc3c3cc(-c4ccc(-n5c6ccccc6c6ccccc65)c(-c5nc(-c6ccccc6)nc(-c6ccccc6)n5)c4)ccc32)c(-c2nc(-c3ccccc3)nc(-c3ccccc3)n2)c1 ; N#Cc1ccc(-n2c3ccccc3c3cc(-c4ccc(-n5c6ccccc6c6ccccc65)c(-c5nc(-c6ccccc6)cc(-c6ccccc6)n5)c4)ccc32)c(-c2nc(-c3ccccc3)nc(-c3ccccc3)n2)c1 ; N#Cc1ccc(-n2c3ccccc3c3cc(-c4ccc(-n5c6ccccc6c6ccccc65)c(-c5cc(-c6ccccc6)nc(-c6ccccc6)n5)c4)ccc32)c(-c2nc(-c3ccccc3)nc(-c3ccccc3)n2)c1]

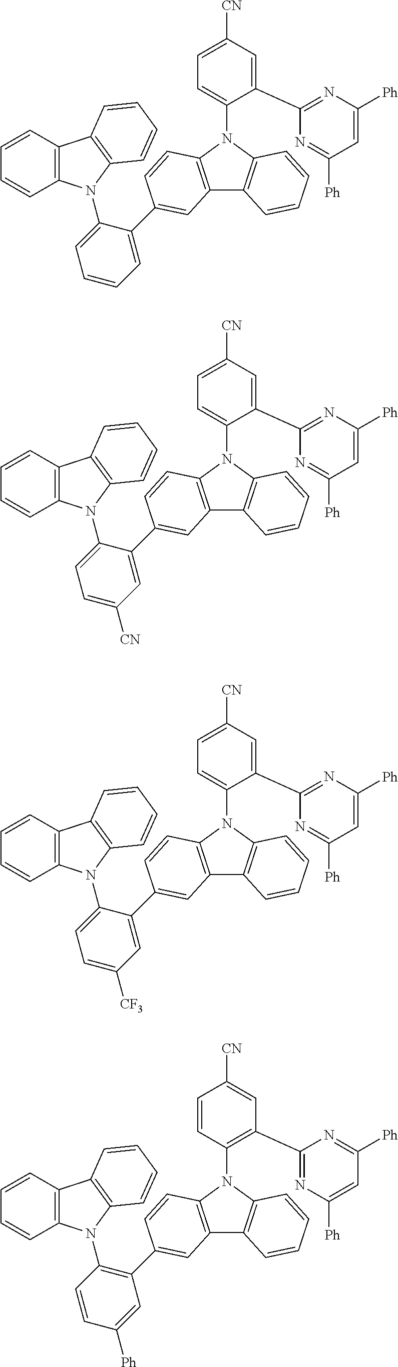 [SMILES: N#Cc1ccc(-n2c3ccccc3c3cc(-c4ccccc4-n4c5ccccc5c5ccccc54)ccc32)c(-c2nc(-c3ccccc3)cc(-c3ccccc3)n2)c1 ; N#Cc1ccc(-n2c3ccccc3c3ccccc32)c(-c2ccc3c(c2)c2ccccc2n3-c2ccc(C#N)cc2-c2nc(-c3ccccc3)cc(-c3ccccc3)n2)c1 ; N#Cc1ccc(-n2c3ccccc3c3cc(-c4cc(C(F)(F)F)ccc4-n4c5ccccc5c5ccccc54)ccc32)c(-c2nc(-c3ccccc3)cc(-c3ccccc3)n2)c1 ; N#Cc1ccc(-n2c3ccccc3c3cc(-c4cc(-c5ccccc5)ccc4-n4c5ccccc5c5ccccc54)ccc32)c(-c2nc(-c3ccccc3)cc(-c3ccccc3)n2)c1]

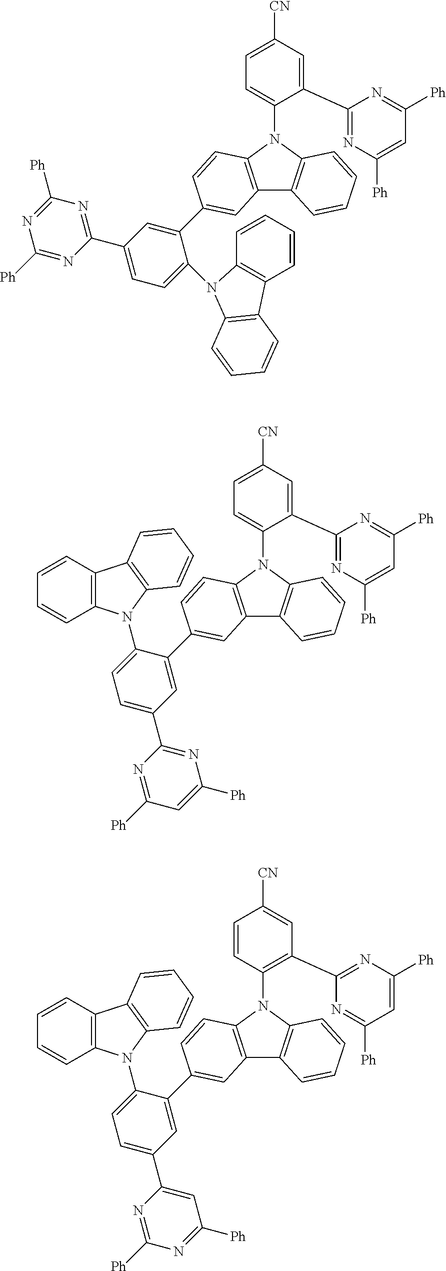 [SMILES: N#Cc1ccc(-n2c3ccccc3c3cc(-c4cc(-c5nc(-c6ccccc6)nc(-c6ccccc6)n5)ccc4-n4c5ccccc5c5ccccc54)ccc32)c(-c2nc(-c3ccccc3)cc(-c3ccccc3)n2)c1 ; N#Cc1ccc(-n2c3ccccc3c3cc(-c4cc(-c5nc(-c6ccccc6)cc(-c6ccccc6)n5)ccc4-n4c5ccccc5c5ccccc54)ccc32)c(-c2nc(-c3ccccc3)cc(-c3ccccc3)n2)c1 ; N#Cc1ccc(-n2c3ccccc3c3cc(-c4cc(-c5cc(-c6ccccc6)nc(-c6ccccc6)n5)ccc4-n4c5ccccc5c5ccccc54)ccc32)c(-c2nc(-c3ccccc3)cc(-c3ccccc3)n2)c1]

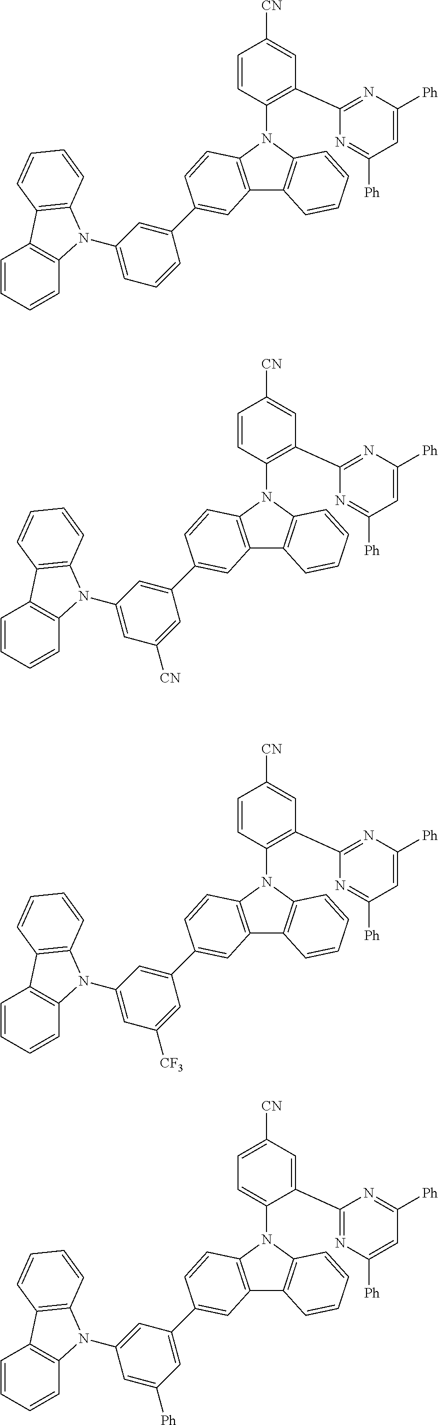 [SMILES: N#Cc1ccc(-n2c3ccccc3c3cc(-c4cccc(-n5c6ccccc6c6ccccc65)c4)ccc32)c(-c2nc(-c3ccccc3)cc(-c3ccccc3)n2)c1 ; N#Cc1cc(-c2ccc3c(c2)c2ccccc2n3-c2ccc(C#N)cc2-c2nc(-c3ccccc3)cc(-c3ccccc3)n2)cc(-n2c3ccccc3c3ccccc32)c1 ; N#Cc1ccc(-n2c3ccccc3c3cc(-c4cc(-n5c6ccccc6c6ccccc65)cc(C(F)(F)F)c4)ccc32)c(-c2nc(-c3ccccc3)cc(-c3ccccc3)n2)c1 ; N#Cc1ccc(-n2c3ccccc3c3cc(-c4cc(-c5ccccc5)cc(-n5c6ccccc6c6ccccc65)c4)ccc32)c(-c2nc(-c3ccccc3)cc(-c3ccccc3)n2)c1]

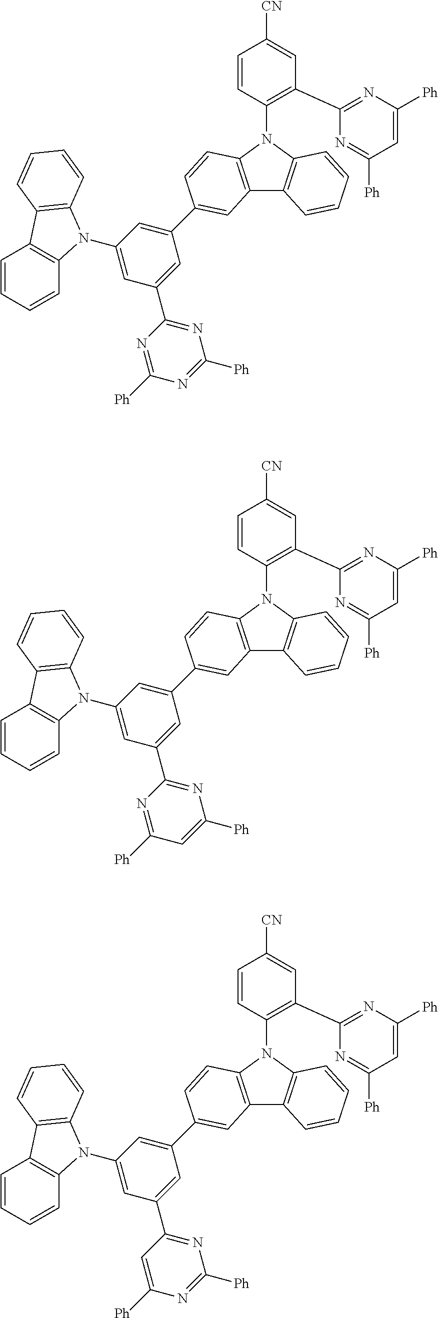 [SMILES: N#Cc1ccc(-n2c3ccccc3c3cc(-c4cc(-c5nc(-c6ccccc6)nc(-c6ccccc6)n5)cc(-n5c6ccccc6c6ccccc65)c4)ccc32)c(-c2nc(-c3ccccc3)cc(-c3ccccc3)n2)c1 ; N#Cc1ccc(-n2c3ccccc3c3cc(-c4cc(-c5nc(-c6ccccc6)cc(-c6ccccc6)n5)cc(-n5c6ccccc6c6ccccc65)c4)ccc32)c(-c2nc(-c3ccccc3)cc(-c3ccccc3)n2)c1 ; N#Cc1ccc(-n2c3ccccc3c3cc(-c4cc(-c5cc(-c6ccccc6)nc(-c6ccccc6)n5)cc(-n5c6ccccc6c6ccccc65)c4)ccc32)c(-c2nc(-c3ccccc3)cc(-c3ccccc3)n2)c1]

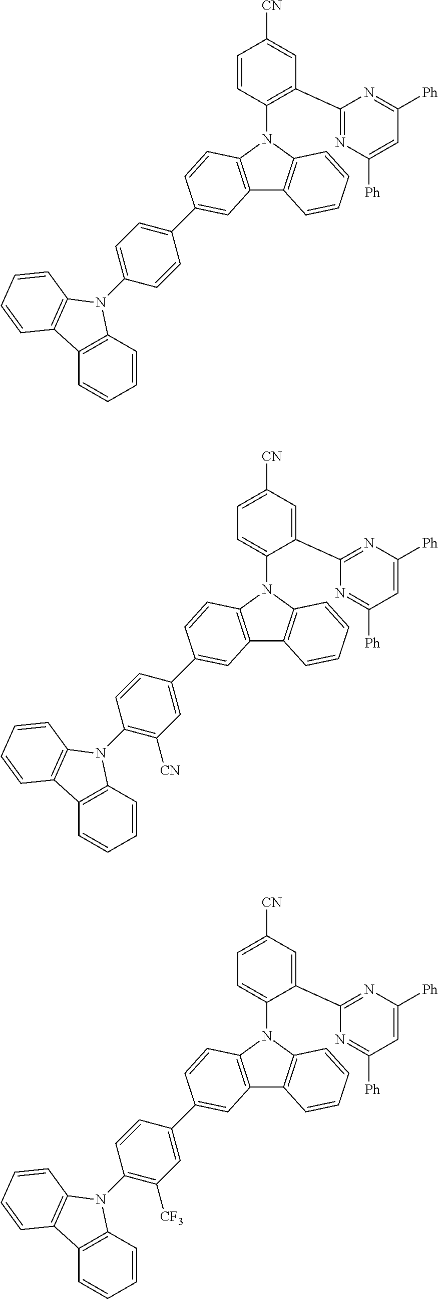 [SMILES: N#Cc1ccc(-n2c3ccccc3c3cc(-c4ccc(-n5c6ccccc6c6ccccc65)cc4)ccc32)c(-c2nc(-c3ccccc3)cc(-c3ccccc3)n2)c1 ; N#Cc1ccc(-n2c3ccccc3c3cc(-c4ccc(-n5c6ccccc6c6ccccc65)c(C#N)c4)ccc32)c(-c2nc(-c3ccccc3)cc(-c3ccccc3)n2)c1 ; N#Cc1ccc(-n2c3ccccc3c3cc(-c4ccc(-n5c6ccccc6c6ccccc65)c(C(F)(F)F)c4)ccc32)c(-c2nc(-c3ccccc3)cc(-c3ccccc3)n2)c1]

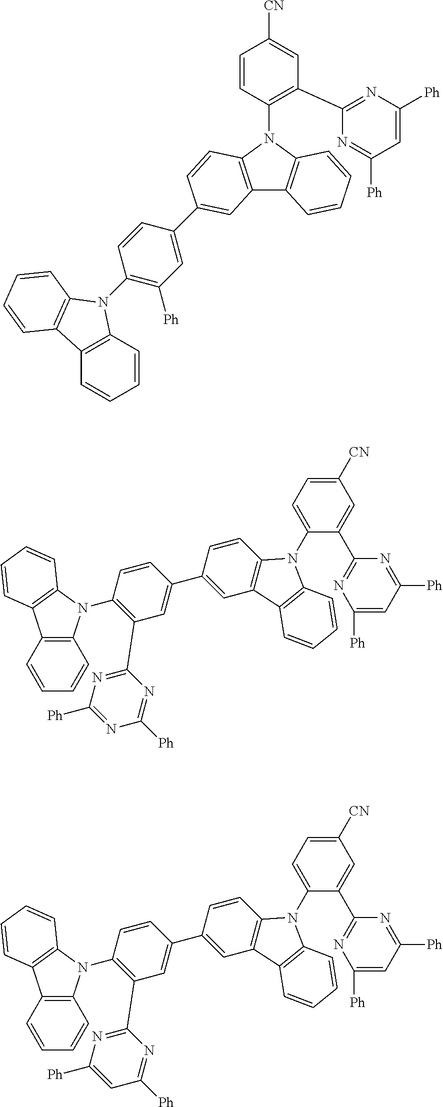 [SMILES: N#Cc1ccc(-n2c3ccccc3c3cc(-c4ccc(-n5c6ccccc6c6ccccc65)c(-c5ccccc5)c4)ccc32)c(-c2nc(-c3ccccc3)cc(-c3ccccc3)n2)c1 ; N#Cc1ccc(-n2c3ccccc3c3cc(-c4ccc(-n5c6ccccc6c6ccccc65)c(-c5nc(-c6ccccc6)nc(-c6ccccc6)n5)c4)ccc32)c(-c2nc(-c3ccccc3)cc(-c3ccccc3)n2)c1 ; N#Cc1ccc(-n2c3ccccc3c3cc(-c4ccc(-n5c6ccccc6c6ccccc65)c(-c5nc(-c6ccccc6)cc(-c6ccccc6)n5)c4)ccc32)c(-c2nc(-c3ccccc3)cc(-c3ccccc3)n2)c1]

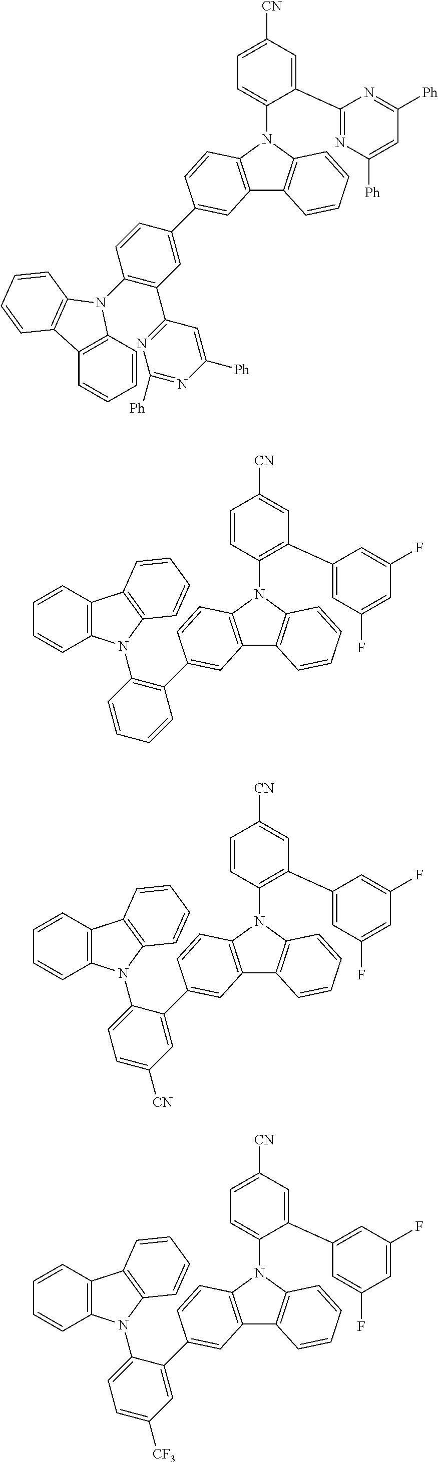 [SMILES: N#Cc1ccc(-n2c3ccccc3c3cc(-c4ccc(-n5c6ccccc6c6ccccc65)c(-c5cc(-c6ccccc6)nc(-c6ccccc6)n5)c4)ccc32)c(-c2nc(-c3ccccc3)cc(-c3ccccc3)n2)c1 ; N#Cc1ccc(-n2c3ccccc3c3cc(-c4ccccc4-n4c5ccccc5c5ccccc54)ccc32)c(-c2cc(F)cc(F)c2)c1 ; N#Cc1ccc(-n2c3ccccc3c3ccccc32)c(-c2ccc3c(c2)c2ccccc2n3-c2ccc(C#N)cc2-c2cc(F)cc(F)c2)c1 ; N#Cc1ccc(-n2c3ccccc3c3cc(-c4cc(C(F)(F)F)ccc4-n4c5ccccc5c5ccccc54)ccc32)c(-c2cc(F)cc(F)c2)c1]

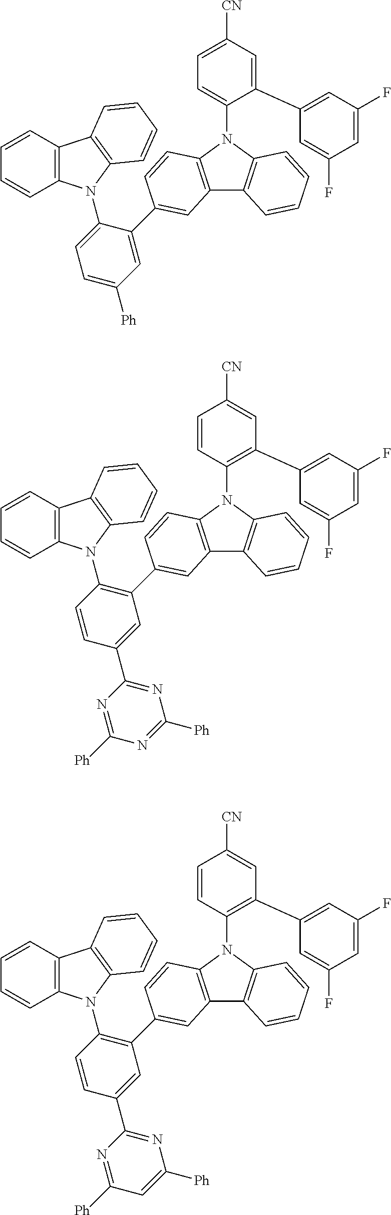 [SMILES: N#Cc1ccc(-n2c3ccccc3c3cc(-c4cc(-c5ccccc5)ccc4-n4c5ccccc5c5ccccc54)ccc32)c(-c2cc(F)cc(F)c2)c1 ; N#Cc1ccc(-n2c3ccccc3c3cc(-c4cc(-c5nc(-c6ccccc6)nc(-c6ccccc6)n5)ccc4-n4c5ccccc5c5ccccc54)ccc32)c(-c2cc(F)cc(F)c2)c1 ; N#Cc1ccc(-n2c3ccccc3c3cc(-c4cc(-c5nc(-c6ccccc6)cc(-c6ccccc6)n5)ccc4-n4c5ccccc5c5ccccc54)ccc32)c(-c2cc(F)cc(F)c2)c1]

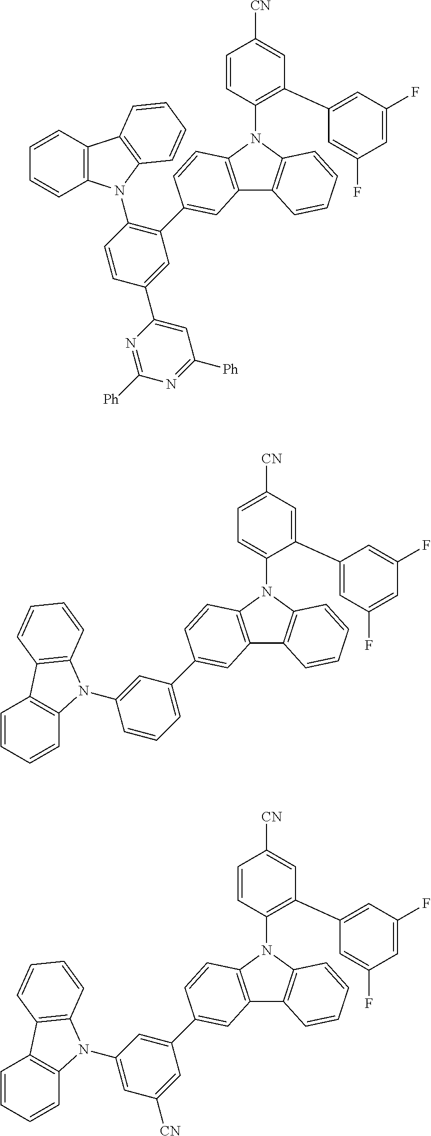 [SMILES: N#Cc1ccc(-n2c3ccccc3c3cc(-c4cc(-c5cc(-c6ccccc6)nc(-c6ccccc6)n5)ccc4-n4c5ccccc5c5ccccc54)ccc32)c(-c2cc(F)cc(F)c2)c1 ; N#Cc1ccc(-n2c3ccccc3c3cc(-c4cccc(-n5c6ccccc6c6ccccc65)c4)ccc32)c(-c2cc(F)cc(F)c2)c1 ; N#Cc1cc(-c2ccc3c(c2)c2ccccc2n3-c2ccc(C#N)cc2-c2cc(F)cc(F)c2)cc(-n2c3ccccc3c3ccccc32)c1]

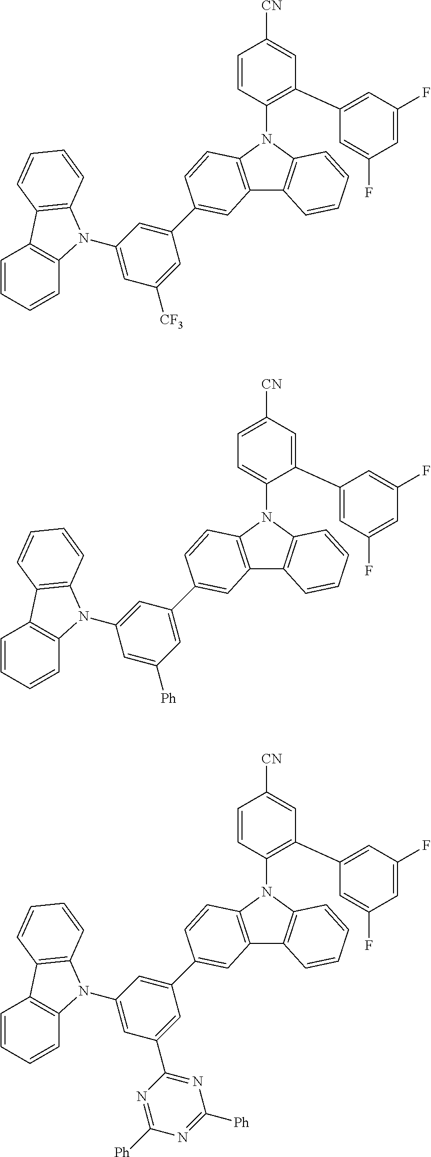 [SMILES: N#Cc1ccc(-n2c3ccccc3c3cc(-c4cc(-n5c6ccccc6c6ccccc65)cc(C(F)(F)F)c4)ccc32)c(-c2cc(F)cc(F)c2)c1 ; N#Cc1ccc(-n2c3ccccc3c3cc(-c4cc(-c5ccccc5)cc(-n5c6ccccc6c6ccccc65)c4)ccc32)c(-c2cc(F)cc(F)c2)c1 ; N#Cc1ccc(-n2c3ccccc3c3cc(-c4cc(-c5nc(-c6ccccc6)nc(-c6ccccc6)n5)cc(-n5c6ccccc6c6ccccc65)c4)ccc32)c(-c2cc(F)cc(F)c2)c1]

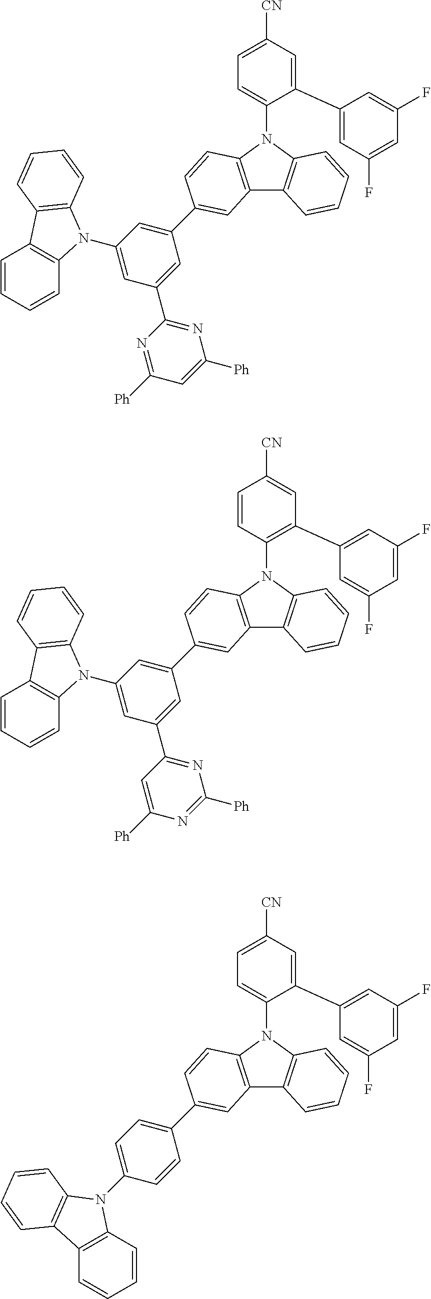 [SMILES: N#Cc1ccc(-n2c3ccccc3c3cc(-c4cc(-c5nc(-c6ccccc6)cc(-c6ccccc6)n5)cc(-n5c6ccccc6c6ccccc65)c4)ccc32)c(-c2cc(F)cc(F)c2)c1 ; N#Cc1ccc(-n2c3ccccc3c3cc(-c4cc(-c5cc(-c6ccccc6)nc(-c6ccccc6)n5)cc(-n5c6ccccc6c6ccccc65)c4)ccc32)c(-c2cc(F)cc(F)c2)c1 ; N#Cc1ccc(-n2c3ccccc3c3cc(-c4ccc(-n5c6ccccc6c6ccccc65)cc4)ccc32)c(-c2cc(F)cc(F)c2)c1]

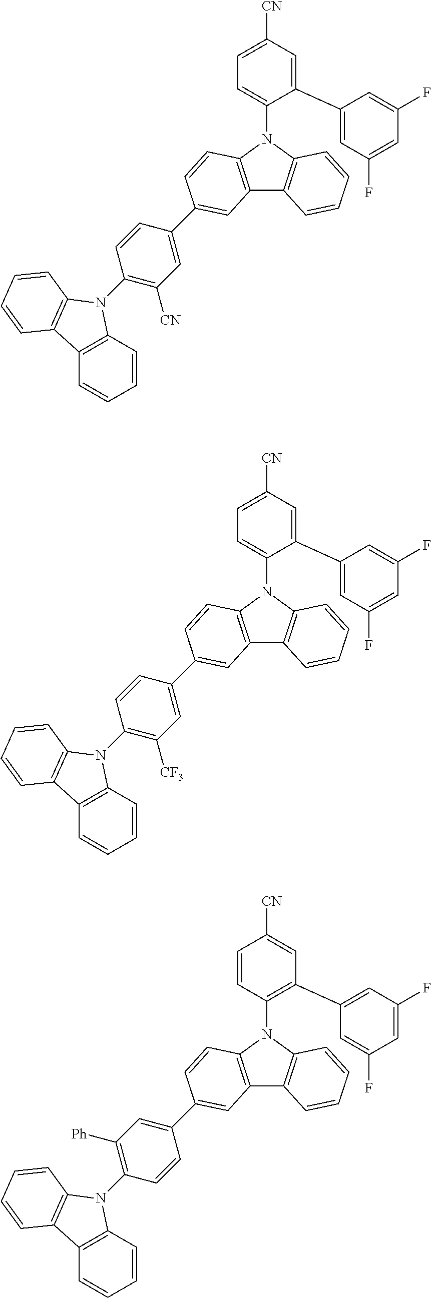 [SMILES: N#Cc1ccc(-n2c3ccccc3c3cc(-c4ccc(-n5c6ccccc6c6ccccc65)c(C#N)c4)ccc32)c(-c2cc(F)cc(F)c2)c1 ; N#Cc1ccc(-n2c3ccccc3c3cc(-c4ccc(-n5c6ccccc6c6ccccc65)c(C(F)(F)F)c4)ccc32)c(-c2cc(F)cc(F)c2)c1 ; N#Cc1ccc(-n2c3ccccc3c3cc(-c4ccc(-n5c6ccccc6c6ccccc65)c(-c5ccccc5)c4)ccc32)c(-c2cc(F)cc(F)c2)c1]

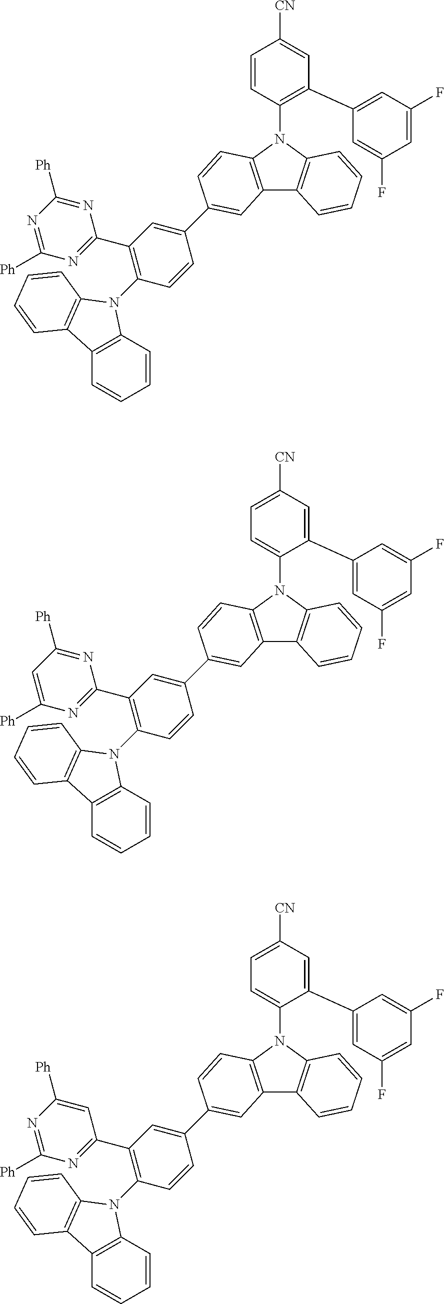 [SMILES: N#Cc1ccc(-n2c3ccccc3c3cc(-c4ccc(-n5c6ccccc6c6ccccc65)c(-c5nc(-c6ccccc6)nc(-c6ccccc6)n5)c4)ccc32)c(-c2cc(F)cc(F)c2)c1 ; N#Cc1ccc(-n2c3ccccc3c3cc(-c4ccc(-n5c6ccccc6c6ccccc65)c(-c5nc(-c6ccccc6)cc(-c6ccccc6)n5)c4)ccc32)c(-c2cc(F)cc(F)c2)c1 ; N#Cc1ccc(-n2c3ccccc3c3cc(-c4ccc(-n5c6ccccc6c6ccccc65)c(-c5cc(-c6ccccc6)nc(-c6ccccc6)n5)c4)ccc32)c(-c2cc(F)cc(F)c2)c1]

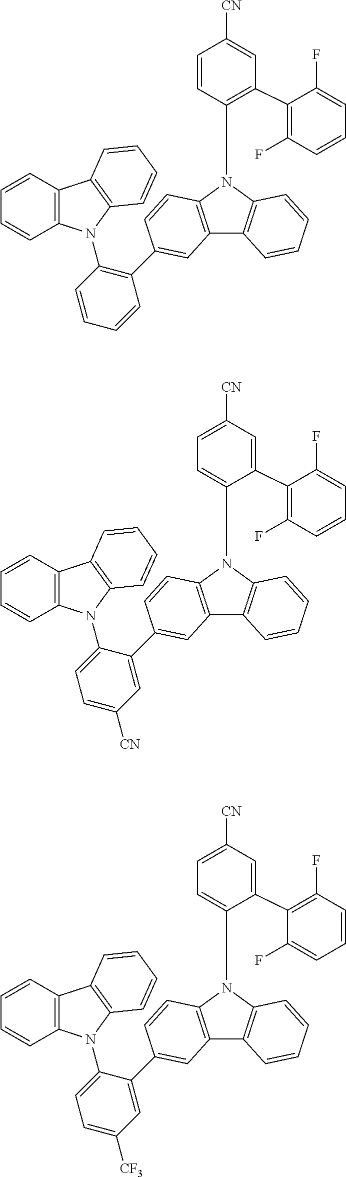 [SMILES: N#Cc1ccc(-n2c3ccccc3c3cc(-c4ccccc4-n4c5ccccc5c5ccccc54)ccc32)c(-c2c(F)cccc2F)c1 ; N#Cc1ccc(-n2c3ccccc3c3ccccc32)c(-c2ccc3c(c2)c2ccccc2n3-c2ccc(C#N)cc2-c2c(F)cccc2F)c1 ; N#Cc1ccc(-n2c3ccccc3c3cc(-c4cc(C(F)(F)F)ccc4-n4c5ccccc5c5ccccc54)ccc32)c(-c2c(F)cccc2F)c1]

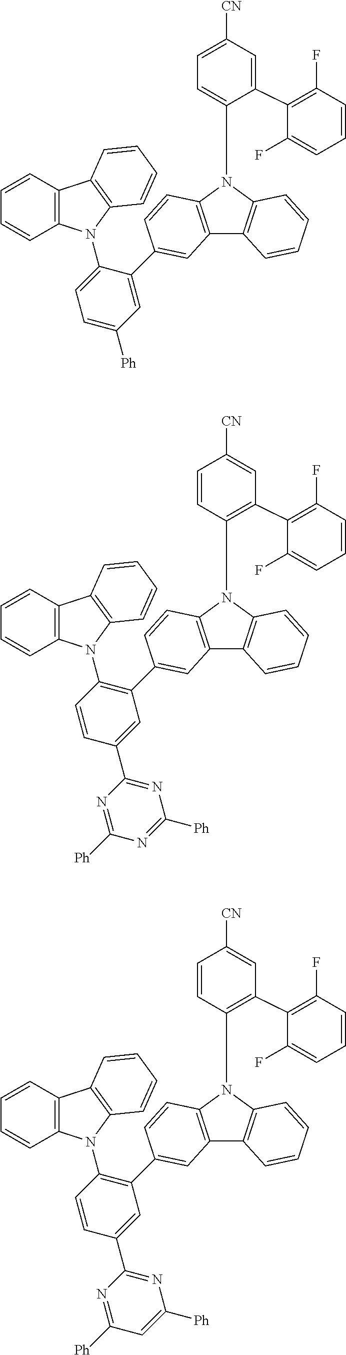 [SMILES: N#Cc1ccc(-n2c3ccccc3c3cc(-c4cc(-c5ccccc5)ccc4-n4c5ccccc5c5ccccc54)ccc32)c(-c2c(F)cccc2F)c1 ; N#Cc1ccc(-n2c3ccccc3c3cc(-c4cc(-c5nc(-c6ccccc6)nc(-c6ccccc6)n5)ccc4-n4c5ccccc5c5ccccc54)ccc32)c(-c2c(F)cccc2F)c1 ; N#Cc1ccc(-n2c3ccccc3c3cc(-c4cc(-c5nc(-c6ccccc6)cc(-c6ccccc6)n5)ccc4-n4c5ccccc5c5ccccc54)ccc32)c(-c2c(F)cccc2F)c1]

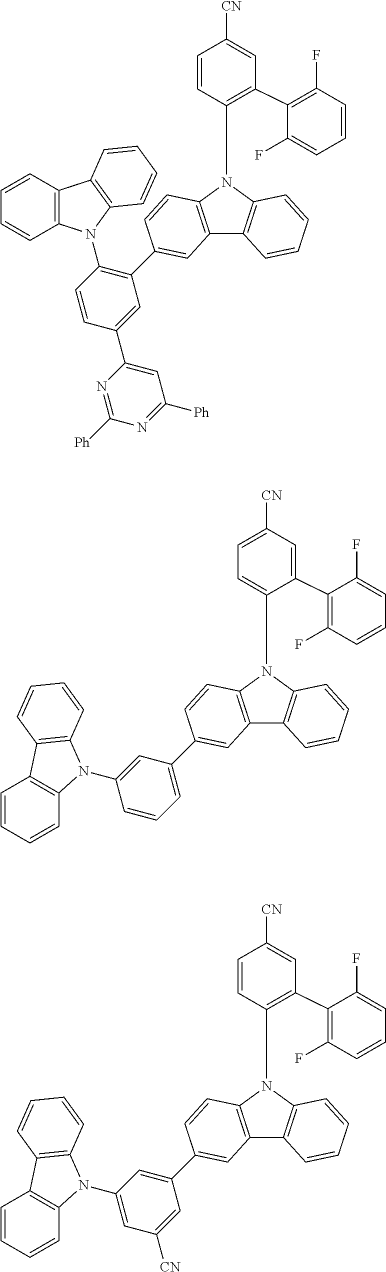 [SMILES: N#Cc1ccc(-n2c3ccccc3c3cc(-c4cc(-c5cc(-c6ccccc6)nc(-c6ccccc6)n5)ccc4-n4c5ccccc5c5ccccc54)ccc32)c(-c2c(F)cccc2F)c1 ; N#Cc1ccc(-n2c3ccccc3c3cc(-c4cccc(-n5c6ccccc6c6ccccc65)c4)ccc32)c(-c2c(F)cccc2F)c1 ; N#Cc1cc(-c2ccc3c(c2)c2ccccc2n3-c2ccc(C#N)cc2-c2c(F)cccc2F)cc(-n2c3ccccc3c3ccccc32)c1]

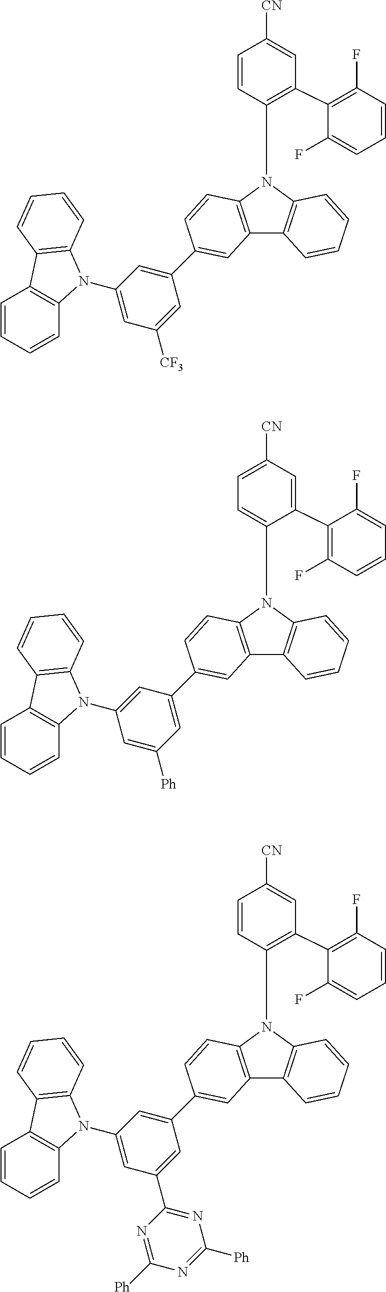 [SMILES: N#Cc1ccc(-n2c3ccccc3c3cc(-c4cc(-n5c6ccccc6c6ccccc65)cc(C(F)(F)F)c4)ccc32)c(-c2c(F)cccc2F)c1 ; N#Cc1ccc(-n2c3ccccc3c3cc(-c4cc(-c5ccccc5)cc(-n5c6ccccc6c6ccccc65)c4)ccc32)c(-c2c(F)cccc2F)c1 ; N#Cc1ccc(-n2c3ccccc3c3cc(-c4cc(-c5nc(-c6ccccc6)nc(-c6ccccc6)n5)cc(-n5c6ccccc6c6ccccc65)c4)ccc32)c(-c2c(F)cccc2F)c1]

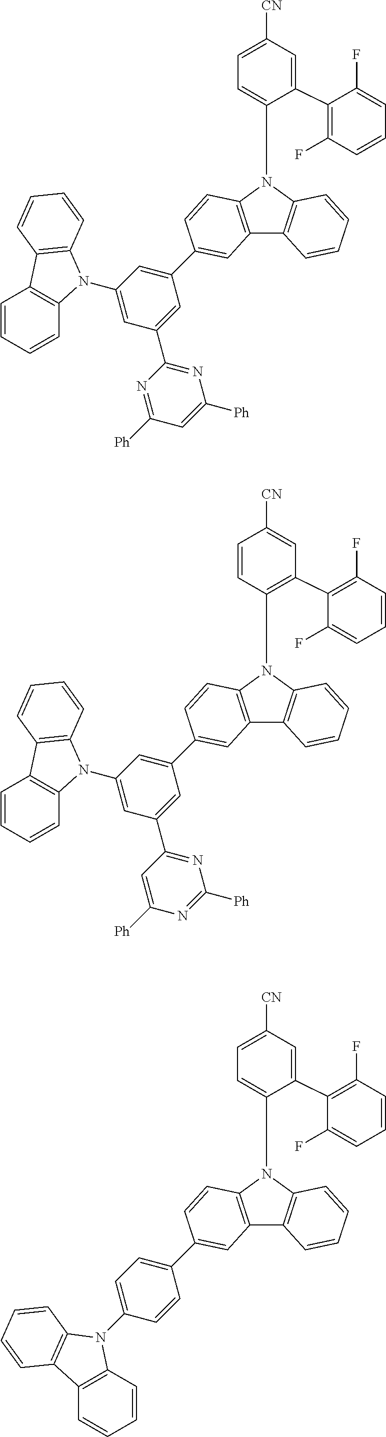 [SMILES: N#Cc1ccc(-n2c3ccccc3c3cc(-c4cc(-c5nc(-c6ccccc6)cc(-c6ccccc6)n5)cc(-n5c6ccccc6c6ccccc65)c4)ccc32)c(-c2c(F)cccc2F)c1 ; N#Cc1ccc(-n2c3ccccc3c3cc(-c4cc(-c5cc(-c6ccccc6)nc(-c6ccccc6)n5)cc(-n5c6ccccc6c6ccccc65)c4)ccc32)c(-c2c(F)cccc2F)c1 ; N#Cc1ccc(-n2c3ccccc3c3cc(-c4ccc(-n5c6ccccc6c6ccccc65)cc4)ccc32)c(-c2c(F)cccc2F)c1]

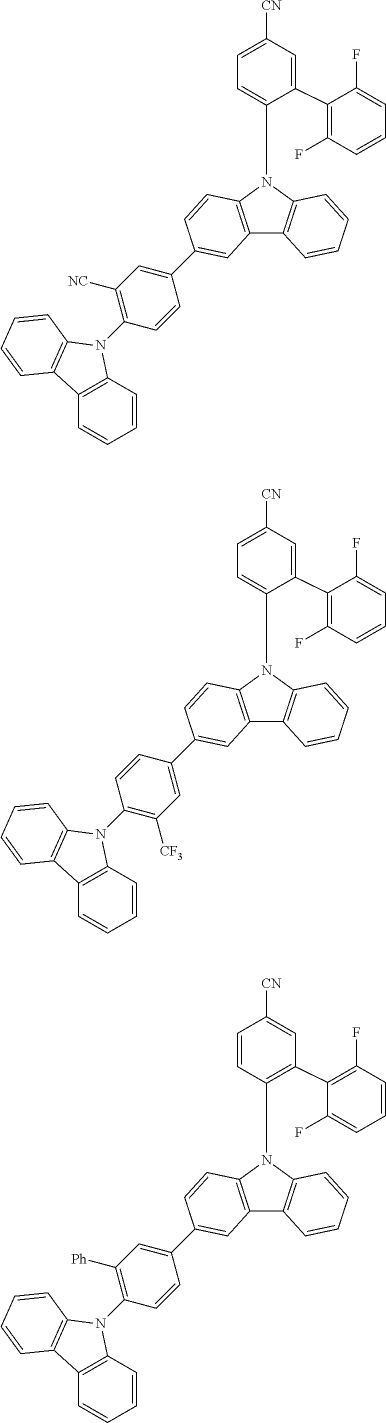 [SMILES: N#Cc1ccc(-n2c3ccccc3c3cc(-c4ccc(-n5c6ccccc6c6ccccc65)c(C#N)c4)ccc32)c(-c2c(F)cccc2F)c1 ; N#Cc1ccc(-n2c3ccccc3c3cc(-c4ccc(-n5c6ccccc6c6ccccc65)c(C(F)(F)F)c4)ccc32)c(-c2c(F)cccc2F)c1 ; N#Cc1ccc(-n2c3ccccc3c3cc(-c4ccc(-n5c6ccccc6c6ccccc65)c(-c5ccccc5)c4)ccc32)c(-c2c(F)cccc2F)c1]

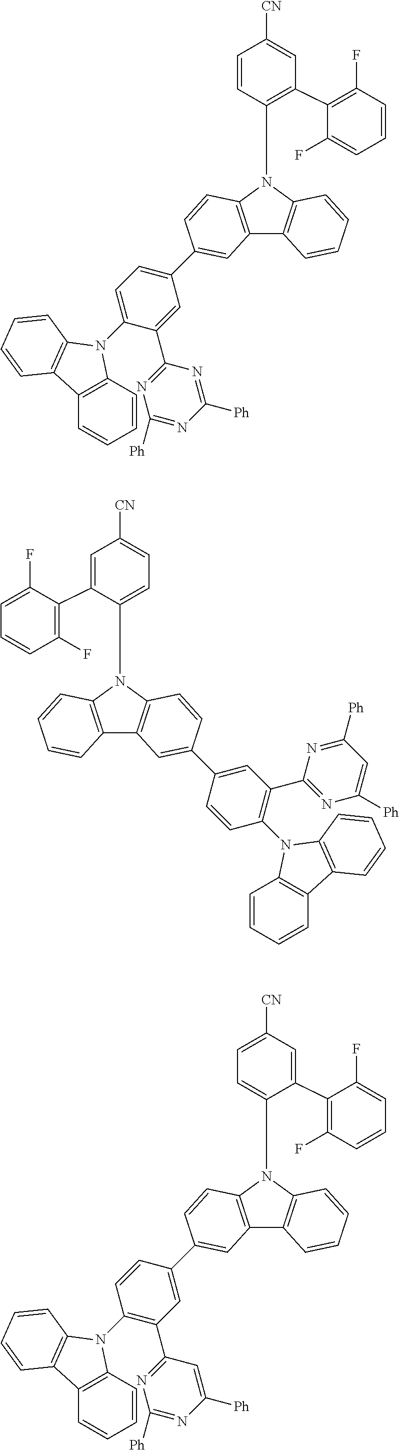 [SMILES: N#Cc1ccc(-n2c3ccccc3c3cc(-c4ccc(-n5c6ccccc6c6ccccc65)c(-c5nc(-c6ccccc6)nc(-c6ccccc6)n5)c4)ccc32)c(-c2c(F)cccc2F)c1 ; N#Cc1ccc(-n2c3ccccc3c3cc(-c4ccc(-n5c6ccccc6c6ccccc65)c(-c5nc(-c6ccccc6)cc(-c6ccccc6)n5)c4)ccc32)c(-c2c(F)cccc2F)c1 ; N#Cc1ccc(-n2c3ccccc3c3cc(-c4ccc(-n5c6ccccc6c6ccccc65)c(-c5cc(-c6ccccc6)nc(-c6ccccc6)n5)c4)ccc32)c(-c2c(F)cccc2F)c1]

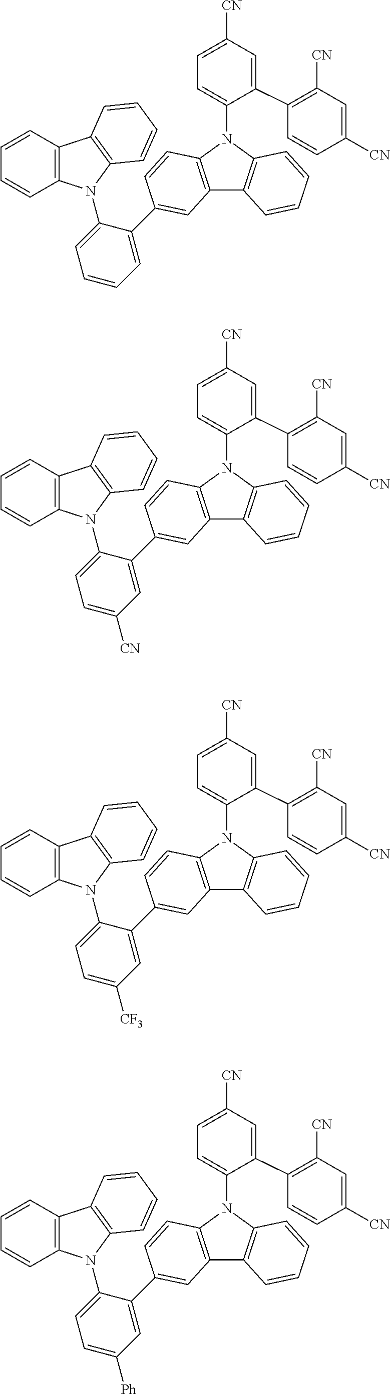 [SMILES: N#Cc1ccc(-c2cc(C#N)ccc2-n2c3ccccc3c3cc(-c4ccccc4-n4c5ccccc5c5ccccc54)ccc32)c(C#N)c1 ; N#Cc1ccc(-c2cc(C#N)ccc2-n2c3ccccc3c3cc(-c4cc(C#N)ccc4-n4c5ccccc5c5ccccc54)ccc32)c(C#N)c1 ; N#Cc1ccc(-c2cc(C#N)ccc2-n2c3ccccc3c3cc(-c4cc(C(F)(F)F)ccc4-n4c5ccccc5c5ccccc54)ccc32)c(C#N)c1 ; N#Cc1ccc(-c2cc(C#N)ccc2-n2c3ccccc3c3cc(-c4cc(-c5ccccc5)ccc4-n4c5ccccc5c5ccccc54)ccc32)c(C#N)c1]

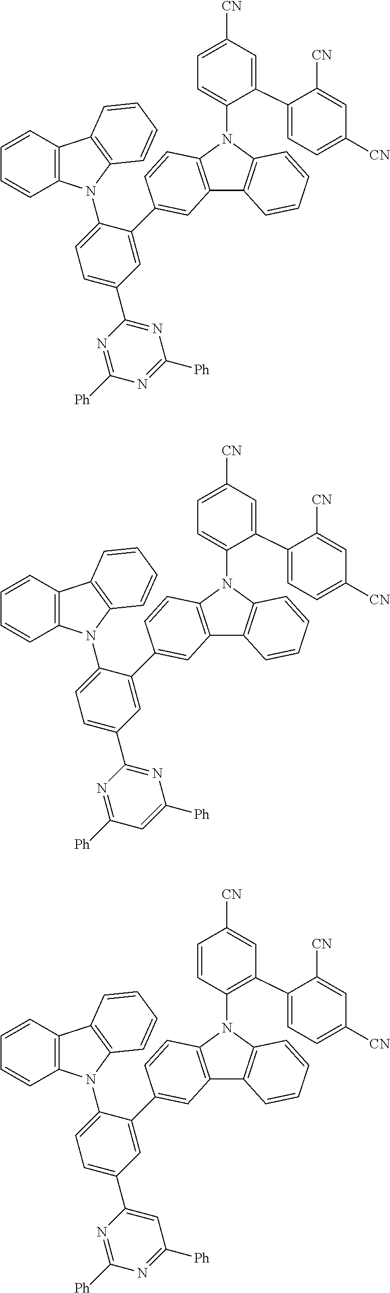 [SMILES: N#Cc1ccc(-c2cc(C#N)ccc2-n2c3ccccc3c3cc(-c4cc(-c5nc(-c6ccccc6)nc(-c6ccccc6)n5)ccc4-n4c5ccccc5c5ccccc54)ccc32)c(C#N)c1 ; N#Cc1ccc(-c2cc(C#N)ccc2-n2c3ccccc3c3cc(-c4cc(-c5nc(-c6ccccc6)cc(-c6ccccc6)n5)ccc4-n4c5ccccc5c5ccccc54)ccc32)c(C#N)c1 ; N#Cc1ccc(-c2cc(C#N)ccc2-n2c3ccccc3c3cc(-c4cc(-c5cc(-c6ccccc6)nc(-c6ccccc6)n5)ccc4-n4c5ccccc5c5ccccc54)ccc32)c(C#N)c1]

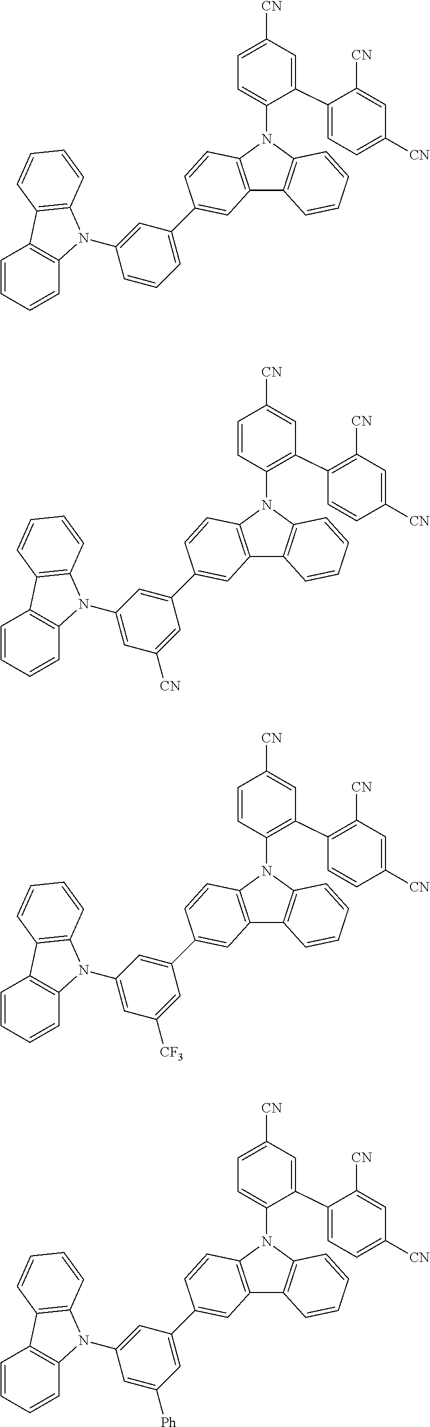 [SMILES: N#Cc1ccc(-c2cc(C#N)ccc2-n2c3ccccc3c3cc(-c4cccc(-n5c6ccccc6c6ccccc65)c4)ccc32)c(C#N)c1 ; N#Cc1cc(-c2ccc3c(c2)c2ccccc2n3-c2ccc(C#N)cc2-c2ccc(C#N)cc2C#N)cc(-n2c3ccccc3c3ccccc32)c1 ; N#Cc1ccc(-c2cc(C#N)ccc2-n2c3ccccc3c3cc(-c4cc(-n5c6ccccc6c6ccccc65)cc(C(F)(F)F)c4)ccc32)c(C#N)c1 ; N#Cc1ccc(-c2cc(C#N)ccc2-n2c3ccccc3c3cc(-c4cc(-c5ccccc5)cc(-n5c6ccccc6c6ccccc65)c4)ccc32)c(C#N)c1]

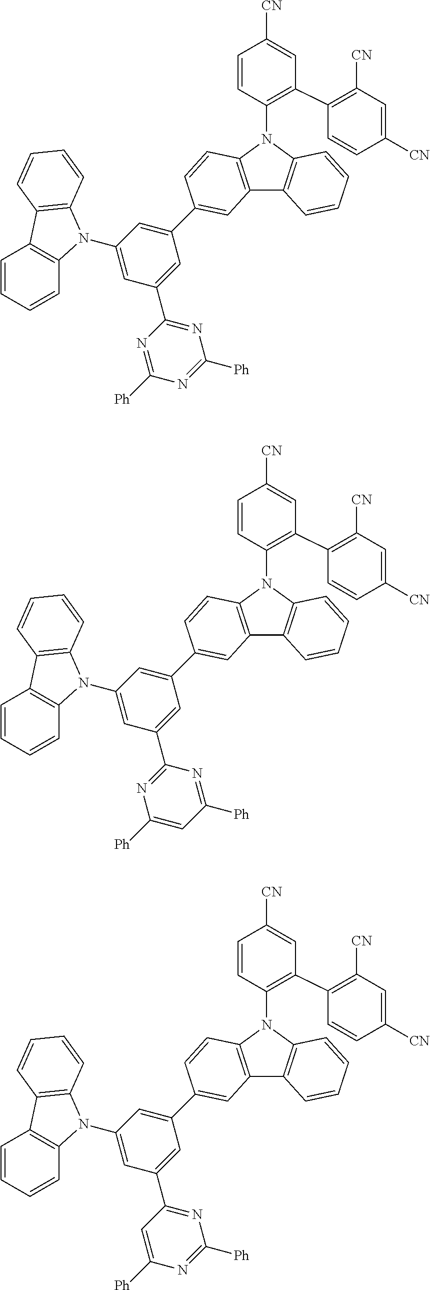 [SMILES: N#Cc1ccc(-c2cc(C#N)ccc2-n2c3ccccc3c3cc(-c4cc(-c5nc(-c6ccccc6)nc(-c6ccccc6)n5)cc(-n5c6ccccc6c6ccccc65)c4)ccc32)c(C#N)c1 ; N#Cc1ccc(-c2cc(C#N)ccc2-n2c3ccccc3c3cc(-c4cc(-c5nc(-c6ccccc6)cc(-c6ccccc6)n5)cc(-n5c6ccccc6c6ccccc65)c4)ccc32)c(C#N)c1 ; N#Cc1ccc(-c2cc(C#N)ccc2-n2c3ccccc3c3cc(-c4cc(-c5cc(-c6ccccc6)nc(-c6ccccc6)n5)cc(-n5c6ccccc6c6ccccc65)c4)ccc32)c(C#N)c1]

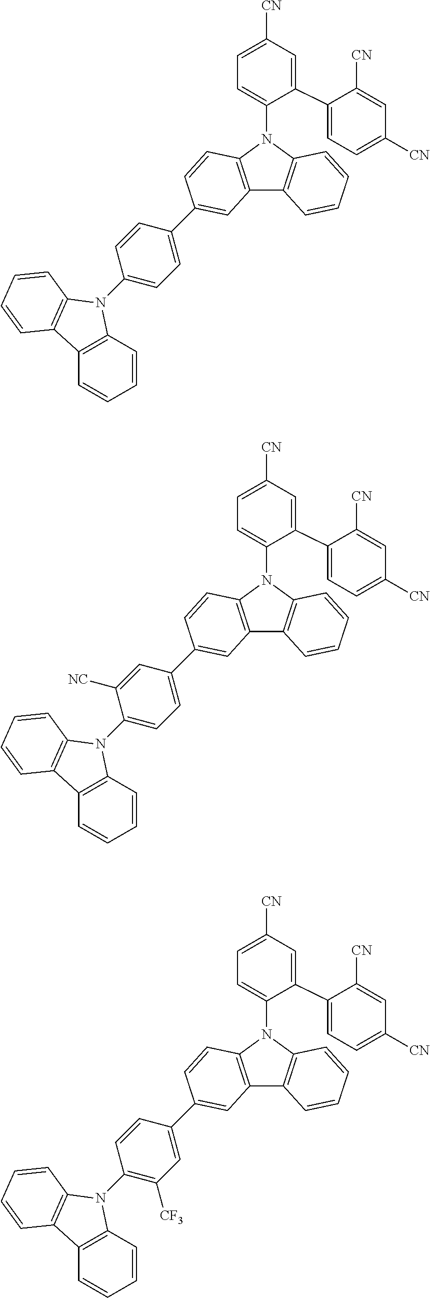 [SMILES: N#Cc1ccc(-c2cc(C#N)ccc2-n2c3ccccc3c3cc(-c4ccc(-n5c6ccccc6c6ccccc65)cc4)ccc32)c(C#N)c1 ; N#Cc1ccc(-c2cc(C#N)ccc2-n2c3ccccc3c3cc(-c4ccc(-n5c6ccccc6c6ccccc65)c(C#N)c4)ccc32)c(C#N)c1 ; N#Cc1ccc(-c2cc(C#N)ccc2-n2c3ccccc3c3cc(-c4ccc(-n5c6ccccc6c6ccccc65)c(C(F)(F)F)c4)ccc32)c(C#N)c1]

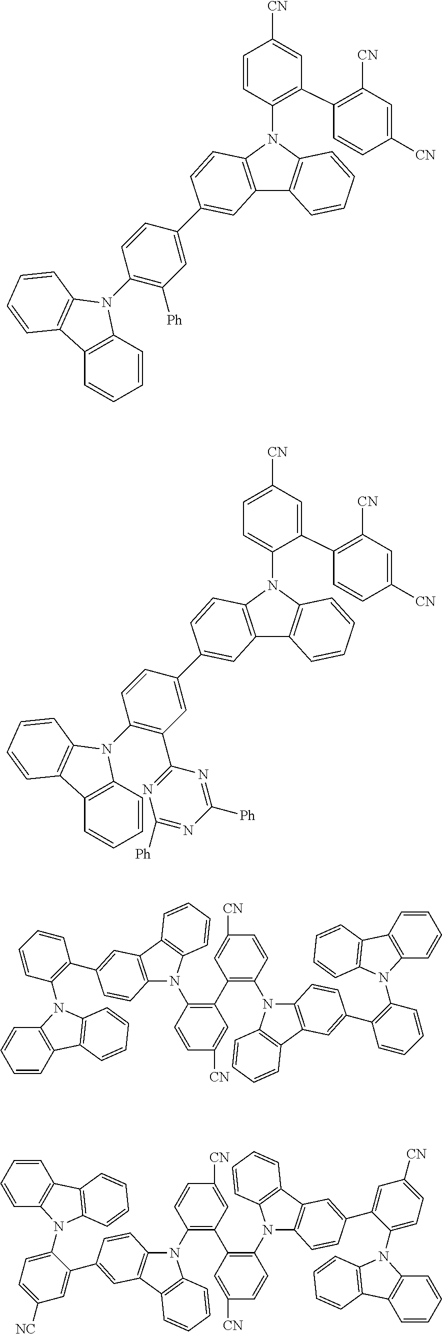 [SMILES: N#Cc1ccc(-c2cc(C#N)ccc2-n2c3ccccc3c3cc(-c4ccc(-n5c6ccccc6c6ccccc65)c(-c5ccccc5)c4)ccc32)c(C#N)c1 ; N#Cc1ccc(-c2cc(C#N)ccc2-n2c3ccccc3c3cc(-c4ccc(-n5c6ccccc6c6ccccc65)c(-c5nc(-c6ccccc6)nc(-c6ccccc6)n5)c4)ccc32)c(C#N)c1 ; N#Cc1ccc(-n2c3ccccc3c3cc(-c4ccccc4-n4c5ccccc5c5ccccc54)ccc32)c(-c2cc(C#N)ccc2-n2c3ccccc3c3cc(-c4ccccc4-n4c5ccccc5c5ccccc54)ccc32)c1 ; N#Cc1ccc(-n2c3ccccc3c3ccccc32)c(-c2ccc3c(c2)c2ccccc2n3-c2ccc(C#N)cc2-c2cc(C#N)ccc2-n2c3ccccc3c3cc(-c4cc(C#N)ccc4-n4c5ccccc5c5ccccc54)ccc32)c1]

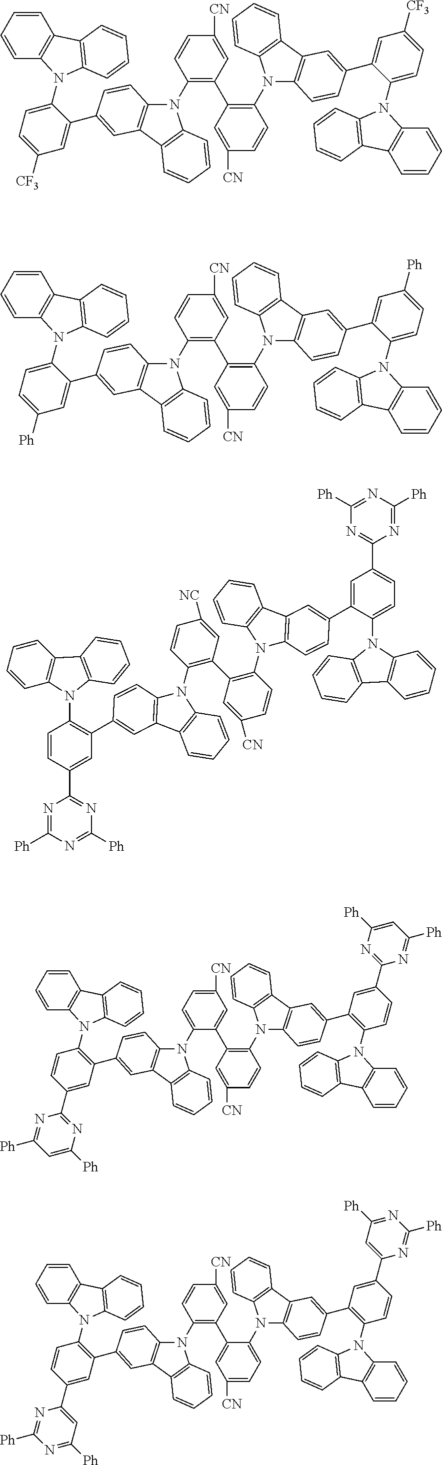

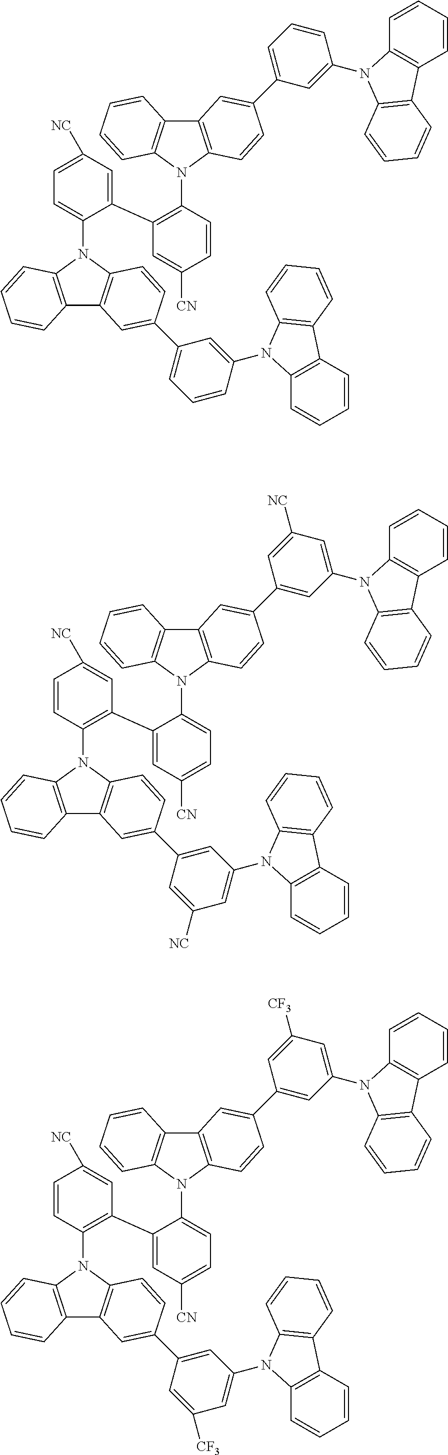 [SMILES: N#Cc1ccc(-n2c3ccccc3c3cc(-c4cccc(-n5c6ccccc6c6ccccc65)c4)ccc32)c(-c2cc(C#N)ccc2-n2c3ccccc3c3cc(-c4cccc(-n5c6ccccc6c6ccccc65)c4)ccc32)c1 ; N#Cc1cc(-c2ccc3c(c2)c2ccccc2n3-c2ccc(C#N)cc2-c2cc(C#N)ccc2-n2c3ccccc3c3cc(-c4cc(C#N)cc(-n5c6ccccc6c6ccccc65)c4)ccc32)cc(-n2c3ccccc3c3ccccc32)c1 ; N#Cc1ccc(-n2c3ccccc3c3cc(-c4cc(-n5c6ccccc6c6ccccc65)cc(C(F)(F)F)c4)ccc32)c(-c2cc(C#N)ccc2-n2c3ccccc3c3cc(-c4cc(-n5c6ccccc6c6ccccc65)cc(C(F)(F)F)c4)ccc32)c1]

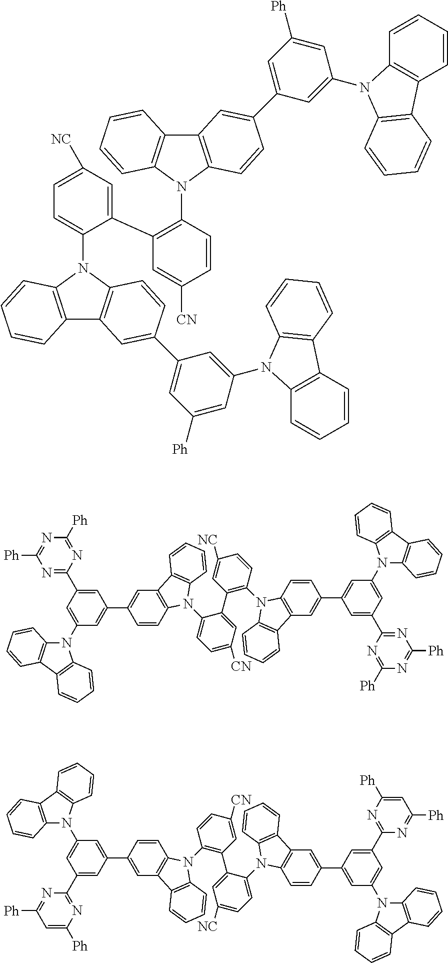

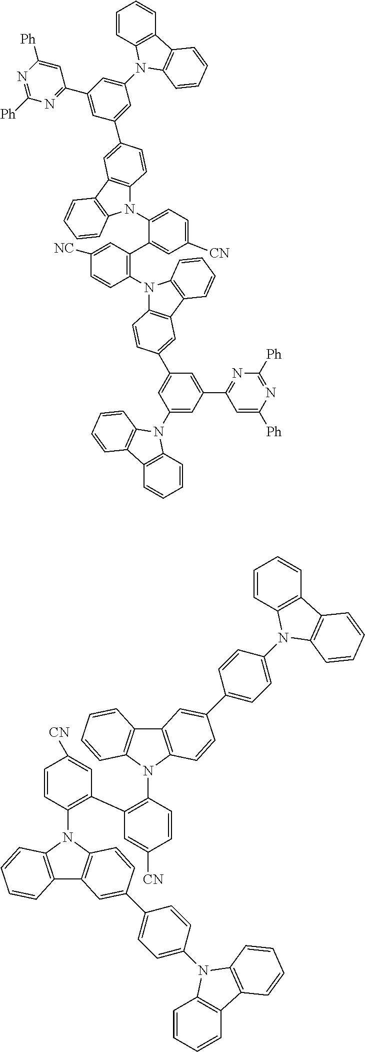 [SMILES: N#Cc1ccc(-n2c3ccccc3c3cc(-c4cc(-c5cc(-c6ccccc6)nc(-c6ccccc6)n5)cc(-n5c6ccccc6c6ccccc65)c4)ccc32)c(-c2cc(C#N)ccc2-n2c3ccccc3c3cc(-c4cc(-c5cc(-c6ccccc6)nc(-c6ccccc6)n5)cc(-n5c6ccccc6c6ccccc65)c4)ccc32)c1 ; N#Cc1ccc(-n2c3ccccc3c3cc(-c4ccc(-n5c6ccccc6c6ccccc65)cc4)ccc32)c(-c2cc(C#N)ccc2-n2c3ccccc3c3cc(-c4ccc(-n5c6ccccc6c6ccccc65)cc4)ccc32)c1]

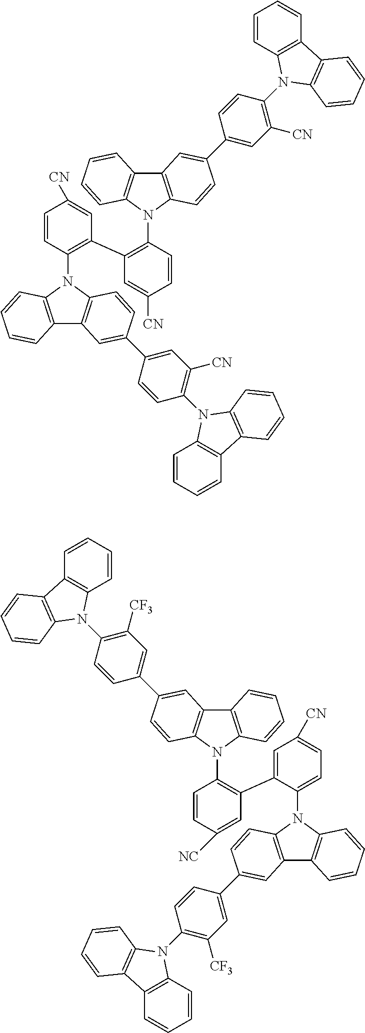 [SMILES: N#Cc1ccc(-n2c3ccccc3c3cc(-c4ccc(-n5c6ccccc6c6ccccc65)c(C#N)c4)ccc32)c(-c2cc(C#N)ccc2-n2c3ccccc3c3cc(-c4ccc(-n5c6ccccc6c6ccccc65)c(C#N)c4)ccc32)c1 ; N#Cc1ccc(-n2c3ccccc3c3cc(-c4ccc(-n5c6ccccc6c6ccccc65)c(C(F)(F)F)c4)ccc32)c(-c2cc(C#N)ccc2-n2c3ccccc3c3cc(-c4ccc(-n5c6ccccc6c6ccccc65)c(C(F)(F)F)c4)ccc32)c1]

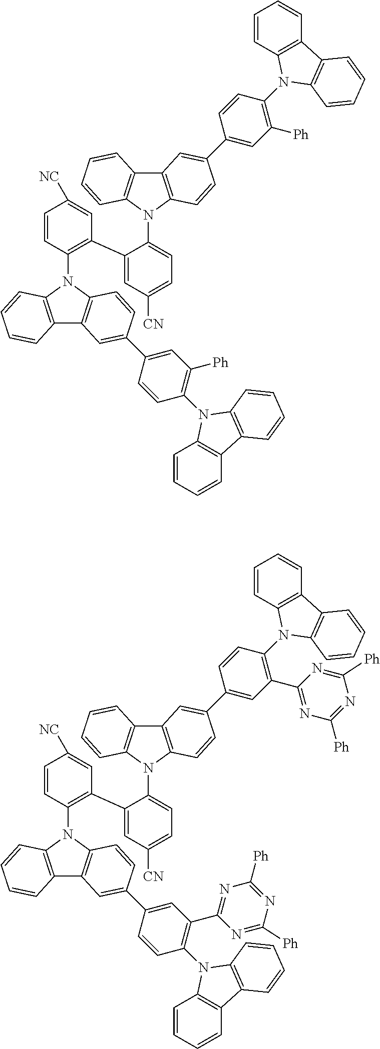 [SMILES: N#Cc1ccc(-n2c3ccccc3c3cc(-c4ccc(-n5c6ccccc6c6ccccc65)c(-c5ccccc5)c4)ccc32)c(-c2cc(C#N)ccc2-n2c3ccccc3c3cc(-c4ccc(-n5c6ccccc6c6ccccc65)c(-c5ccccc5)c4)ccc32)c1 ; N#Cc1ccc(-n2c3ccccc3c3cc(-c4ccc(-n5c6ccccc6c6ccccc65)c(-c5nc(-c6ccccc6)nc(-c6ccccc6)n5)c4)ccc32)c(-c2cc(C#N)ccc2-n2c3ccccc3c3cc(-c4ccc(-n5c6ccccc6c6ccccc65)c(-c5nc(-c6ccccc6)nc(-c6ccccc6)n5)c4)ccc32)c1]

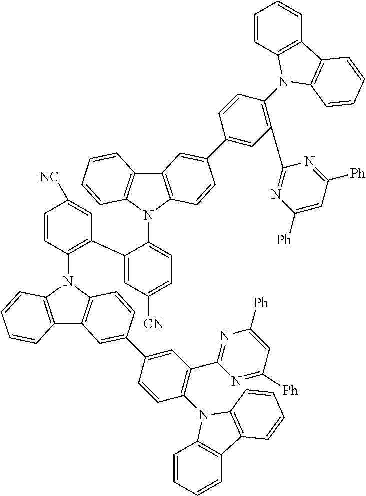 [SMILES: N#Cc1ccc(-n2c3ccccc3c3cc(-c4ccc(-n5c6ccccc6c6ccccc65)c(-c5nc(-c6ccccc6)cc(-c6ccccc6)n5)c4)ccc32)c(-c2cc(C#N)ccc2-n2c3ccccc3c3cc(-c4ccc(-n5c6ccccc6c6ccccc65)c(-c5nc(-c6ccccc6)cc(-c6ccccc6)n5)c4)ccc32)c1]

### FIGURES

The figures show:

FIG. 1 Emission spectrum of Example 1 (10 wt %) in PMMA.

FIG. 2 Emission spectrum of Example 2 (10 wt %) in PMMA.

FIG. 3 Emission spectrum of Example 3 (10 wt %) in PMMA.

FIG. 4 Emission spectrum of Example 4 (10 wt %) in PMMA.

FIG. 5 Emission spectrum of Example 5 (10 wt %) in PMMA.

FIG. 6 Emission spectrum of Example 6 (10 wt %) in PMMA.

FIG. 7 Emission spectrum of Example 7 (10 wt %) in PMMA.

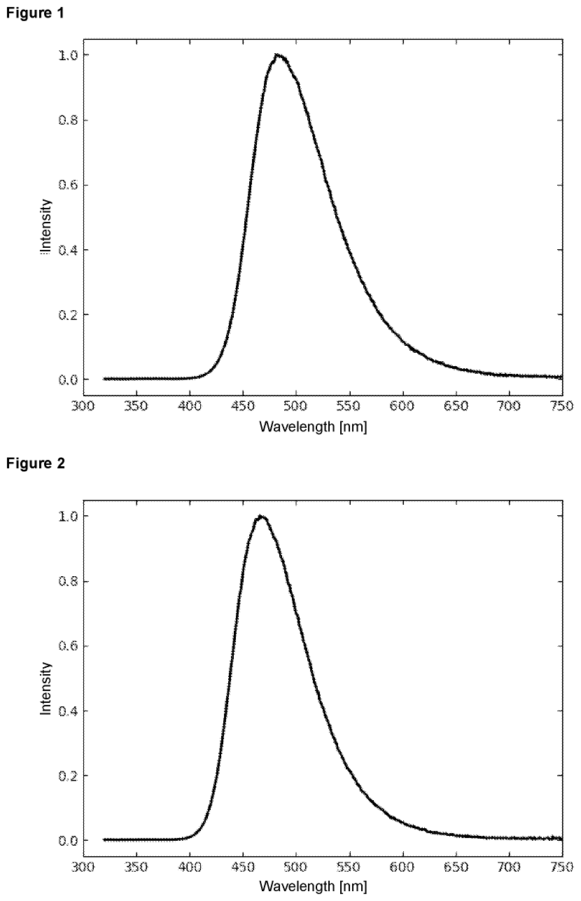

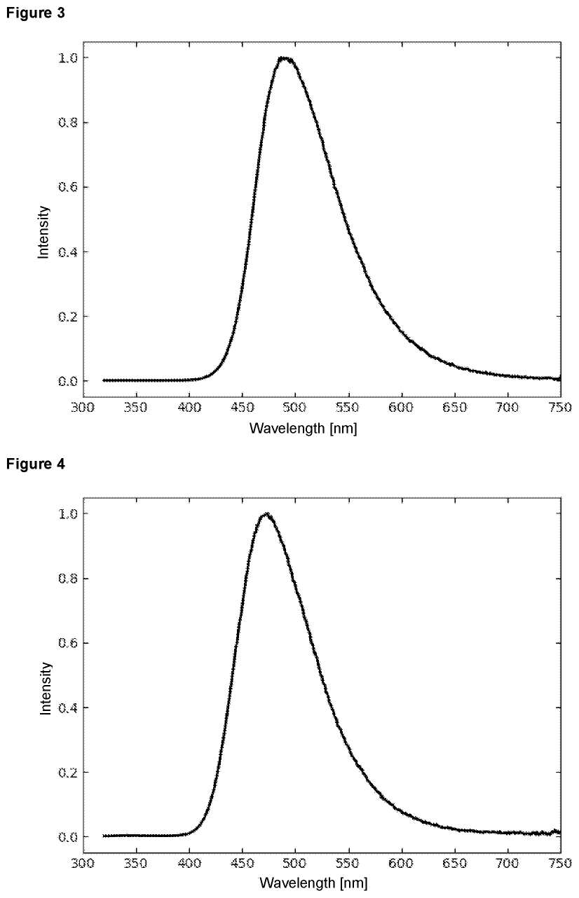

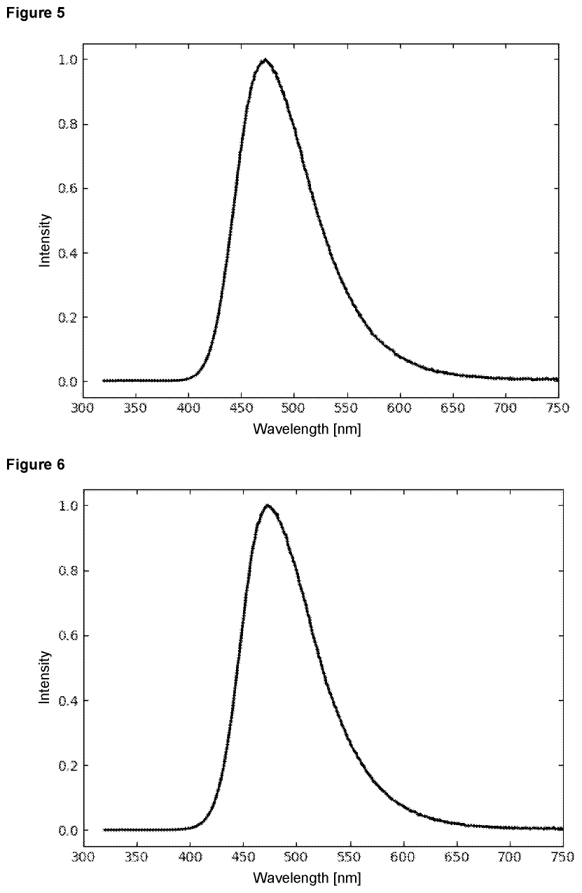

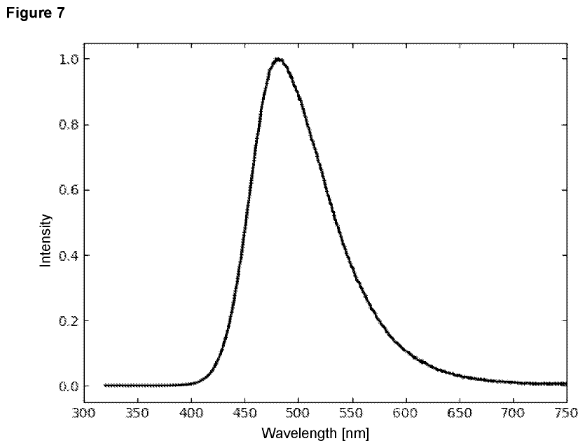

1. An organic molecule comprising a structure of Formula I: 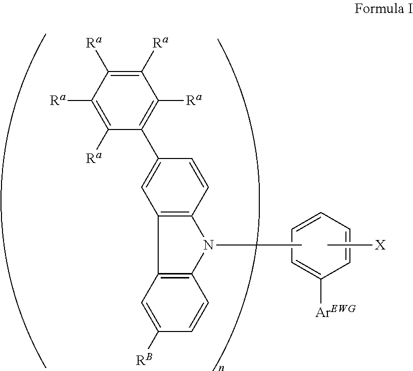
wherein
n is 1 or 2;
X is selected from the group consisting of H, SiMe₃, SiPh₃, CN, and CF₃;
Ar^{EWG} is selected from the group consisting of Formulas IIa to IIo: 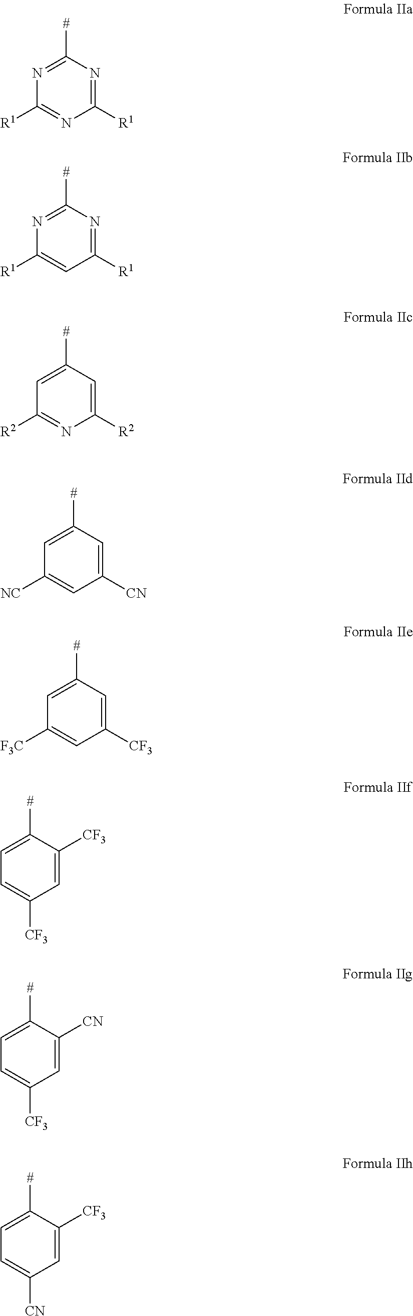 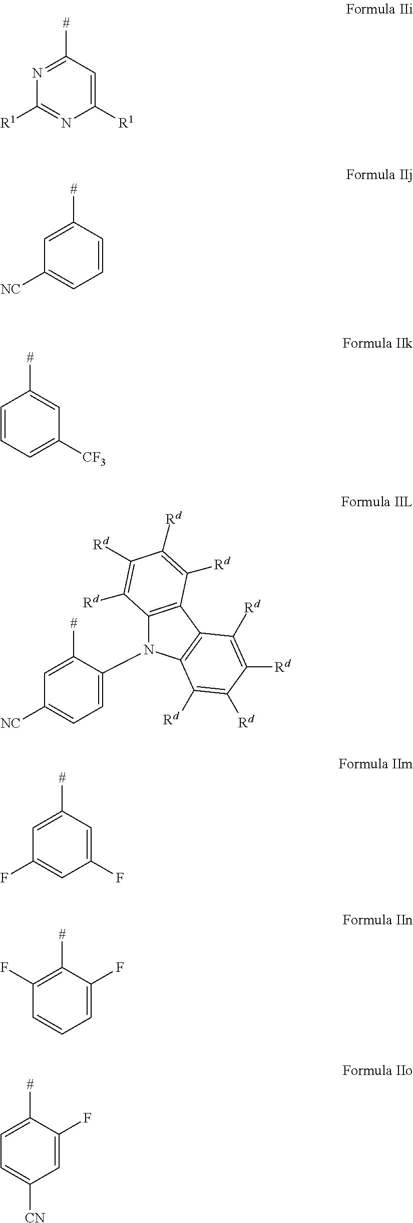
wherein # represents the binding site of the single bond shown in Formula I, which connects Ar^{EWG} to the phenyl ring;
R¹ is selected independently of one another at each occurrence from the group consisting of
hydrogen,
deuterium,
C₁-C₅ alkyl,
wherein one or more hydrogen atoms are optionally substituted by deuterium;
C₂-C₈ alkenyl,
wherein one or more hydrogen atoms are optionally substituted by deuterium;
C₂-C₈ alkynyl,
wherein one or more hydrogen atoms are optionally substituted by deuterium; and
C₆-C₁₈ aryl,
which is optionally substituted with one or more substituents R⁶;
R² is selected independently of one another at each occurrence from the group consisting of
hydrogen,
deuterium,
C₁-C₅ alkyl,
wherein one or more hydrogen atoms are optionally substituted by deuterium;
C₂-C₈ alkenyl,
wherein one or more hydrogen atoms are optionally substituted by deuterium;
C₂-C₈ alkynyl,
wherein one or more hydrogen atoms are optionally substituted by deuterium; and
C₆-C₁₈ aryl,
which is optionally substituted with one or more substituents R⁶;
R^{d} is selected independently of one another at each occurrence from the group consisting of hydrogen, deuterium, N(R⁶)₂, OR⁶, Si(R⁶)₃, B(OR⁶)₂, OSO₂R₆, CF₃, CN, F, Br, I,
C₁-C₄₀ alkyl,
which is optionally substituted with one or more substituents R⁶, and
wherein one or more non-adjacent CH₂ groups are optionally substituted by R⁶C═CR⁶, C═C, Si(R⁶)₂, Ge(R⁶)₂, Sn(R⁶)₂, C═O, C═S, C═Se, C═NR⁶, P(═O)(R⁶), SO, SO₂, NR⁶, O, S, or CONR⁶;
C₁-C₄₀ alkoxy,
which is optionally substituted with one or more substituents R⁶, and
wherein one or more non-adjacent CH₂ groups are optionally substituted by R⁶C═CR⁶, C═C, Si(R⁶)₂, Ge(R⁶)₂, Sn(R⁶)₂, C═O, C═S, C═Se, C═NR⁶, P(═O)(R⁶), SO, SO₂, NR⁶, O, S, or CONR⁶;
C₁-C₄₀ thioalkoxy,
which is optionally substituted with one or more substituents R⁶, and
wherein one or more non-adjacent CH₂ groups are optionally substituted by R⁶C═CR⁶, C═C, Si(R⁶)₂, Ge(R⁶)₂, Sn(R⁶)₂, C═O, C═S, C═Se, C═NR⁶, P(═O)(R⁶), SO, SO₂, NR⁶, O, S, or CONR⁶;
C₂-C₄₀ alkenyl,
which is optionally substituted with one or more substituents R⁶, and
wherein one or more non-adjacent CH₂ groups are optionally substituted by R⁶C═CR⁶, C═C, Si(R⁶)₂, Ge(R⁶)₂, Sn(R⁶)₂, C═O, C═S, C═Se, C═NR⁶, P(═O)(R⁶), SO, SO₂, NR⁶, O, S, or CONR⁶;
C₂-C₄₀ alkynyl,
which is optionally substituted with one or more substituents R⁶, and
wherein one or more non-adjacent CH₂ groups are optionally substituted by R⁶C═CR⁶, C═C, Si(R⁶)₂, Ge(R⁶)₂, Sn(R⁶)₂, C═O, C═S, C═Se, C═NR⁶, P(═O)(R⁶), SO, SO₂, NR⁶, O, S, or CONR⁶;
C₆-C₆₀ aryl,
which is optionally substituted with one or more substituents R⁶; and
C₃-C₅₇ heteroaryl,
which is optionally substituted with one or more substituents R⁶;
R^{a} is selected independently of one another at each occurrence from the group consisting of R^{A},
hydrogen, deuterium, N(R⁶)₂, OR⁶, Si(R⁶)₃, B(OR⁶)₂, OSO₂R⁶, CF₃, CN, F, Br, I,
C₁-C₄₀ alkyl,
which is optionally substituted with one or more substituents R⁶, and
wherein one or more non-adjacent CH₂ groups are optionally substituted by R⁶C═CR⁶, C═C, Si(R⁶)₂, Ge(R⁶)₂, Sn(R⁶)₂, C═O, C═S, C═Se, C═NR⁶, P(═O)(R⁶), SO, SO₂, NR⁶, O, S, or CONR⁶;
C₁-C₄₀ alkoxy,
which is optionally substituted with one or more substituents R⁶, and
wherein one or more non-adjacent CH₂ groups are optionally substituted by R⁶C═CR⁶, C═C, Si(R⁶)₂, Ge(R⁶)₂, Sn(R⁶)₂, C═O, C═S, C═Se, C═NR⁶, P(═O)(R⁶), SO, SO₂, NR⁶, O, S, or CONR⁶;
C₁-C₄₀ thioalkoxy,
which is optionally substituted with one or more substituents R⁶, and
wherein one or more non-adjacent CH₂ groups are optionally substituted by R⁶C═CR⁶, C═C, Si(R⁶)₂, Ge(R⁶)₂, Sn(R⁶)₂, C═O, C═S, C═Se, C═NR⁶, P(═O)(R⁶), SO, SO₂, NR⁶, O, S, or CONR⁶;
C₂-C₄₀ alkenyl,
which is optionally substituted with one or more substituents R⁶, and
wherein one or more non-adjacent CH₂ groups are optionally substituted by R⁶C═CR⁶, C═C, Si(R⁶)₂, Ge(R⁶)₂, Sn(R⁶)₂, C═O, C═S, C═Se, C═NR⁶, P(═O)(R⁶), SO, SO₂, NR⁶, O, S, or CONR⁶;
C₂-C₄₀ alkynyl,
which is optionally substituted with one or more substituents R⁶, and
wherein one or more non-adjacent CH₂ groups are optionally substituted by R⁶C═CR⁶, C═C, Si(R⁶)₂, Ge(R⁶)₂, Sn(R⁶)₂, C═O, C═S, C═Se, C═NR⁶, P(═O)(R⁶), SO, SO₂, NR⁶, O, S, or CONR⁶;
C₆-C₆₀ aryl,
which is optionally substituted with one or more substituents R⁶; and
C₃-C₅₇ heteroaryl,
which is optionally substituted with one or more substituents R⁶;
R⁶ is selected independently of one another at each occurrence from the group consisting of hydrogen, deuterium, OPh, CF₃, CN, F,
C₁-C₅ alkyl,
wherein one or more hydrogen atoms are optionally substituted independently of one another by deuterium, CN, CF₃, or F;
C₁-C₅ alkoxy,
wherein one or more hydrogen atoms are optionally substituted independently of one another by deuterium, CN, CF₃, or F;
C₁-C₅ thioalkoxy,
wherein one or more hydrogen atoms are optionally substituted independently of one another by deuterium, CN, CF₃, or F;
C₂-C₅ alkenyl,
wherein one or more hydrogen atoms are optionally substituted independently of one another by deuterium, CN, CF₃, or F;
C₂-C₅ alkynyl,
wherein one or more hydrogen atoms are optionally substituted independently of one another by deuterium, CN, CF₃, or F;
C₆-C₁₈ aryl,
which is optionally substituted with one or more C₁-C₅ alkyl substituents;
C₃-C₁₇ heteroaryl,
which is optionally substituted with one or more C₁-C₅ alkyl substituents;
N(C₆-C₁₈ aryl)₂;
N(C₃-C₁₇ heteroaryl)₂,
and N(C₃-C₁₇ heteroaryl)(C₆-C₁₈ aryl);
R^{A} is selected independently of one another at each occurrence from a chemical structure of Formula IIA 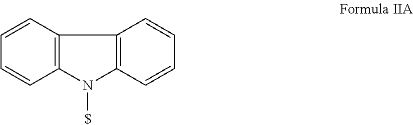
or a chemical structure of Formula IIB 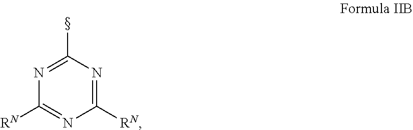
wherein R^{N} is selected from the group consisting of Me, ⁱPr, ^{t}Bu, CN, CF₃, and Ph;
wherein $ represents the binding site of a single bond;
wherein at least one substituent R^{a} is R^{A};
wherein R^{B} is selected from the group consisting of
H,
Ph,
CN,
CF₃,
of a chemical structure of Formula IIA-2 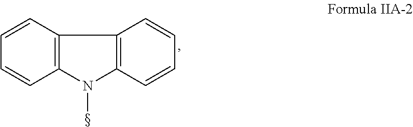
or a chemical structure of Formula IIB-2 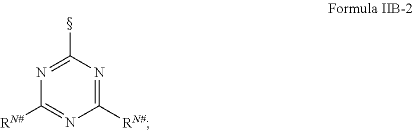
wherein R^{N#} is selected from the group consisting of Me, ⁱPr, ^{t}Bu, CN, CF₃, and Ph;
wherein § represents the binding site of a single bond.

2. The organic molecule according to claim 1, wherein, independently of one another at each occurrence, R¹ is phenyl, which is optionally substituted with one or more substituents R⁶, and
R₂ is independently of one another at each occurrence selected from the group consisting of
hydrogen,
CN,
CF₃, and
phenyl, which is optionally substituted with one or more substituents R⁶.

3. The organic molecule according to claim 1, comprising a structure selected from the group consisting of Formula IIa-1, Formula IIa-2, and Formula IIa-3: 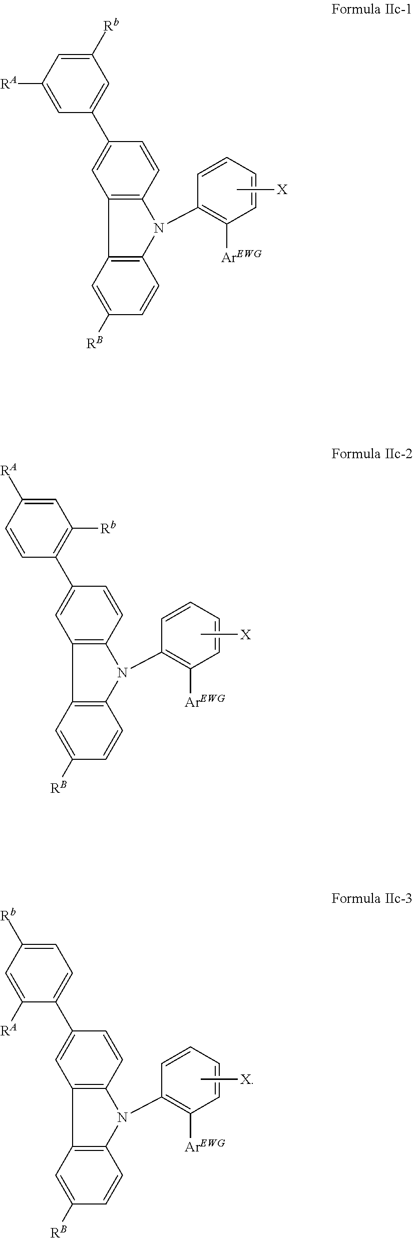
wherein R^{b} is independently of one another at each occurrence selected from the group consisting of
H,
Me,
ⁱPr,
^{t}Bu,
CN,
CF₃,
Ph, which is optionally substituted with one or more substituents independently selected from the group consisting of Me, ⁱPr, ^{t}Bu, CN, CF₃ and Ph,
pyridinyl, which is optionally substituted with one or more substituents independently selected from the group consisting of Me, ⁱPr, ^{t}Bu, CN, CF₃, and Ph,
pyrimidinyl, which is optionally substituted with one or more substituents independently selected from the group consisting of Me, ⁱPr, ^{t}Bu, CN, CF₃, and Ph,
carbazolyl, which is optionally substituted with one or more substituents independently selected from the group consisting of Me, ⁱPr, ^{t}Bu, CN, CF₃, and Ph,
triazinyl, which is optionally substituted with one or more substituents independently selected from the group consisting of Me, ⁱPr, ^{t}Bu, CN, CF₃, and Ph,
and N(Ph)₂.

4. The organic molecule according to claim 1, comprising a structure selected from the groups shown below:

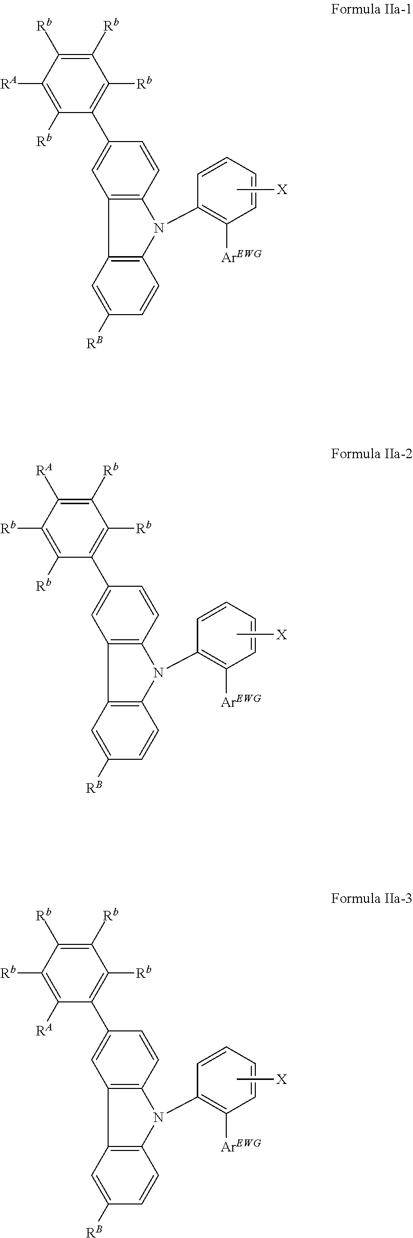

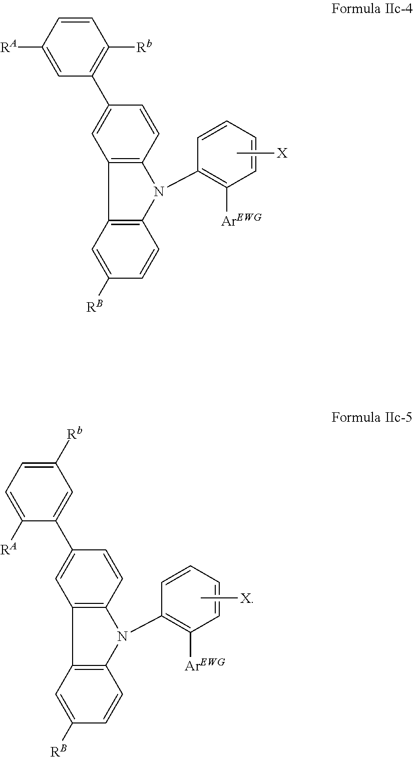

5. The organic molecule according to claim 1, wherein R^{b} is independently at each occurrence selected from the group consisting of H and CN.

6. The organic molecule according to claim 1, comprising or consisting of a structure selected from the groups shown below:

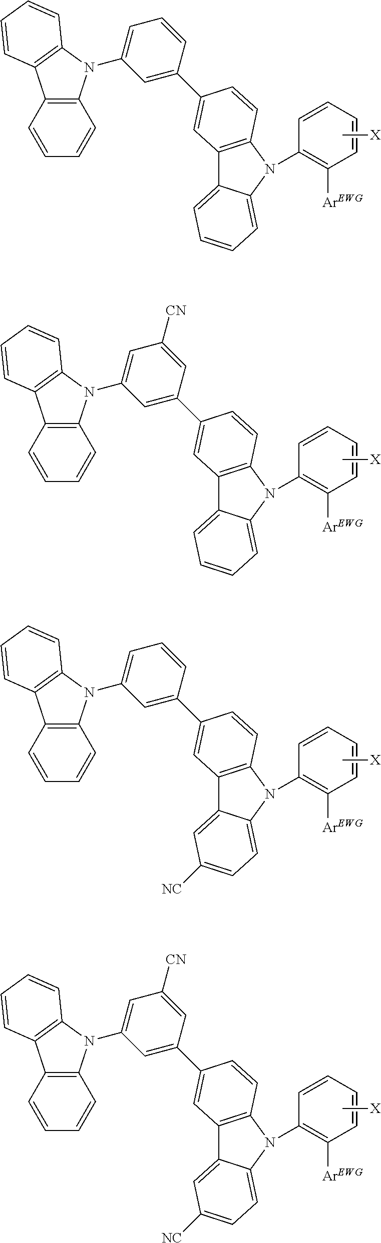

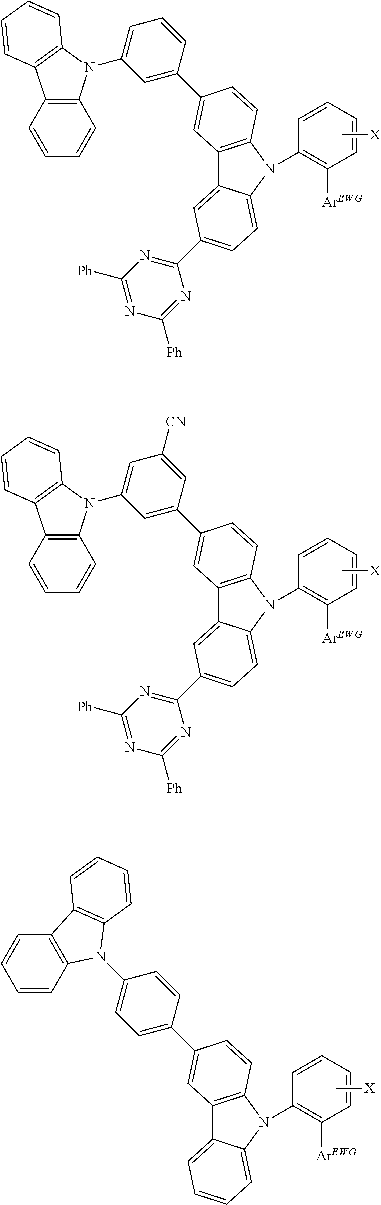

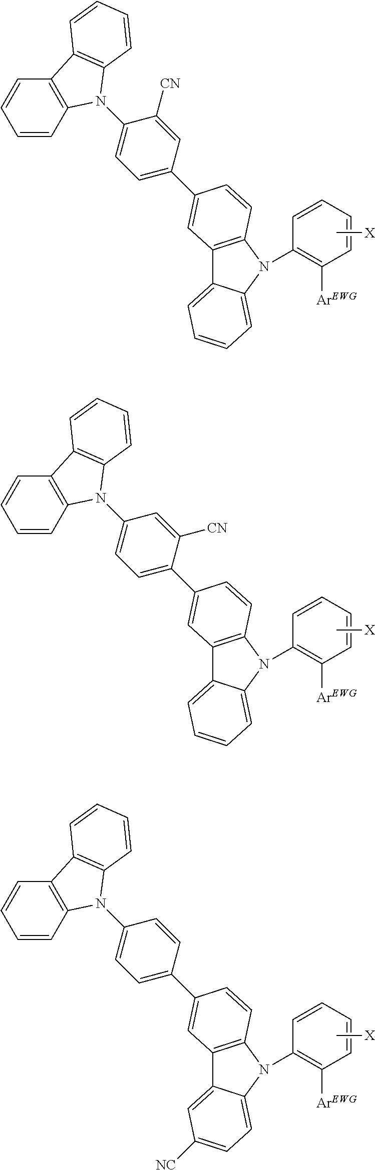

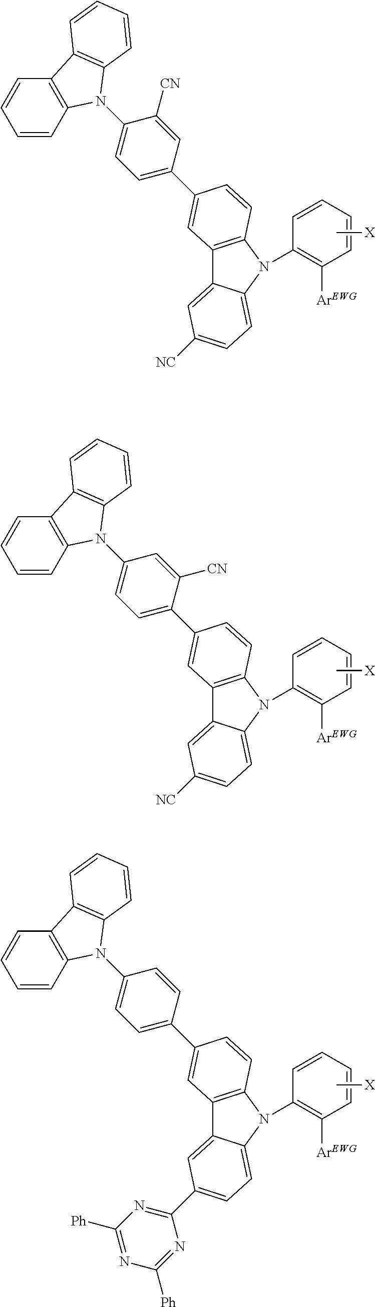

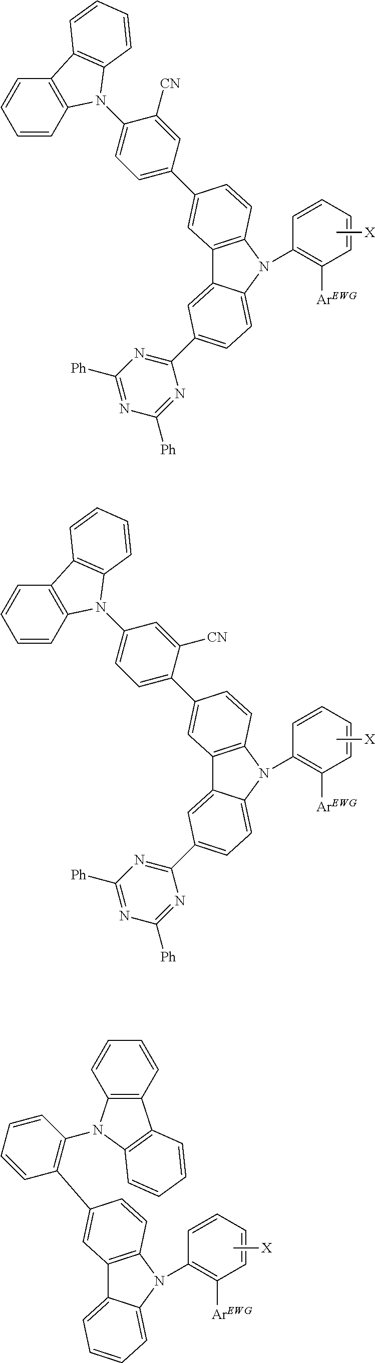

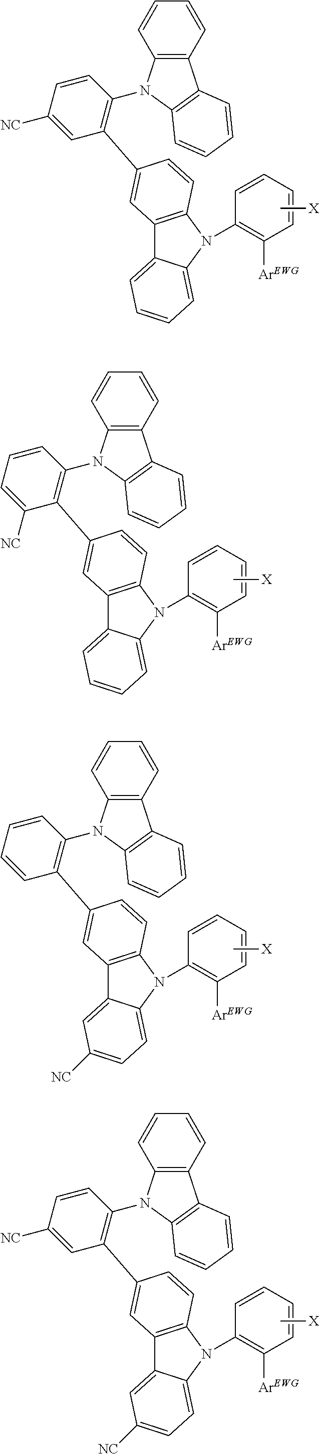

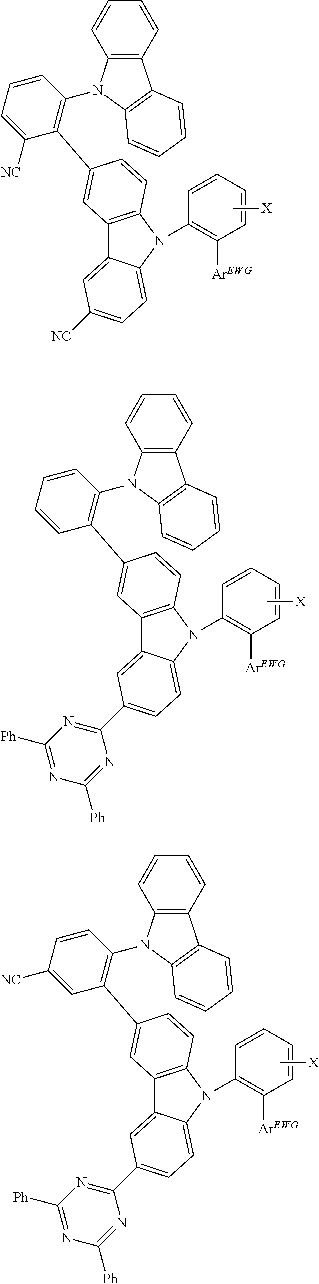

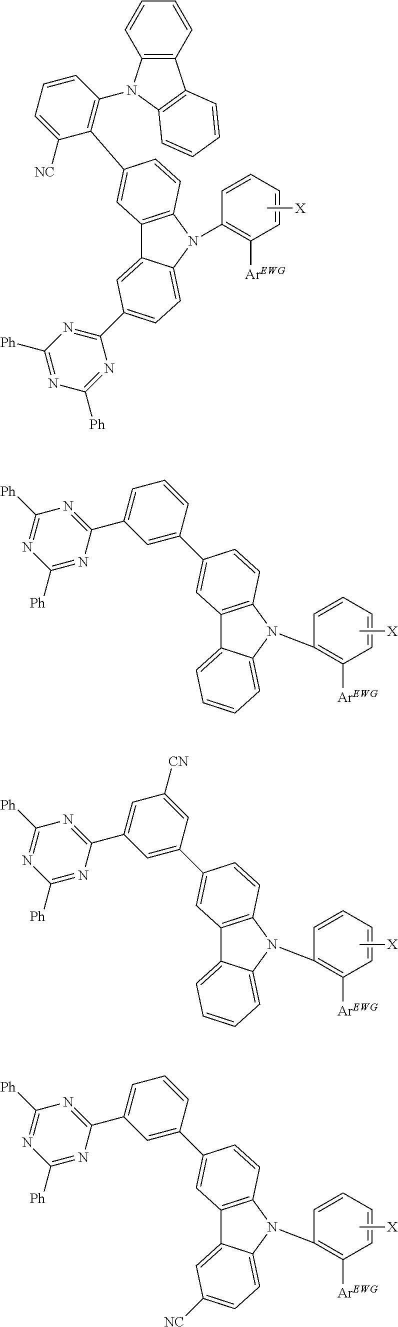

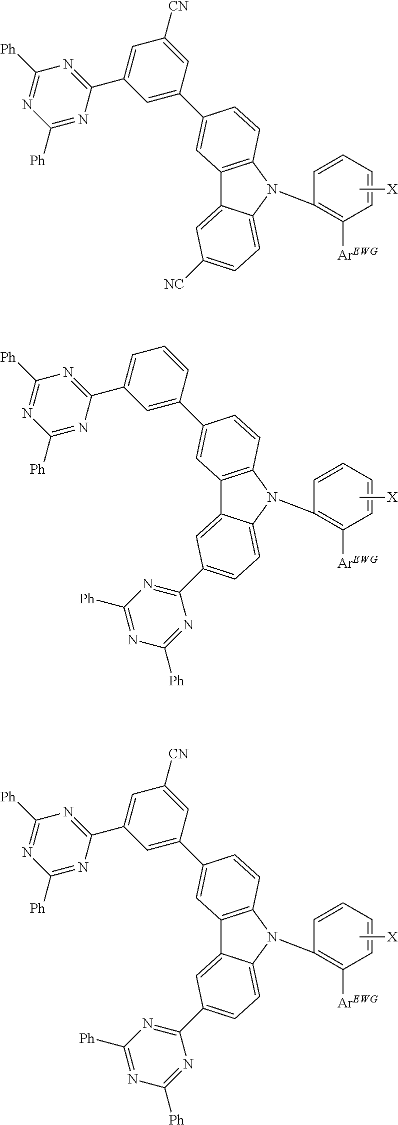

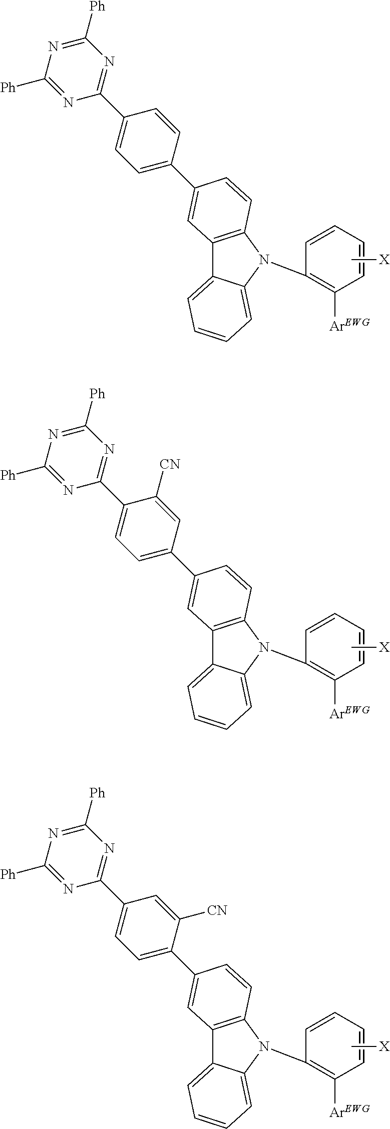

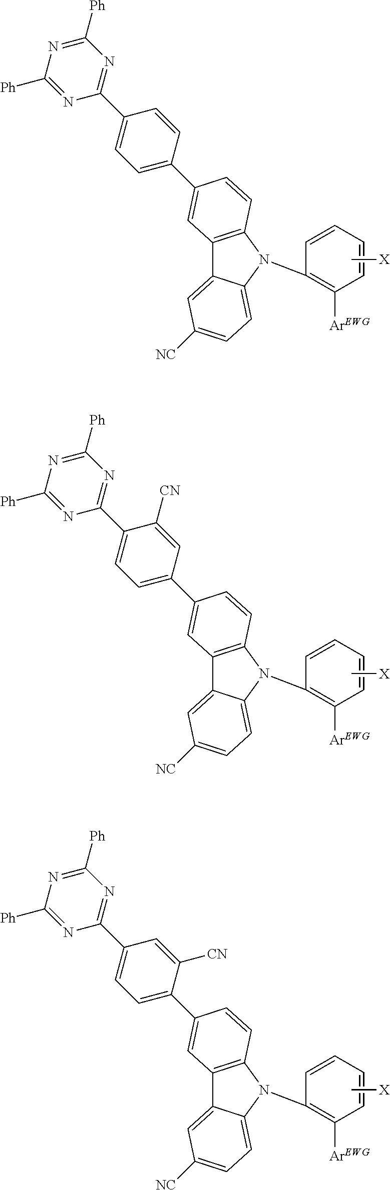

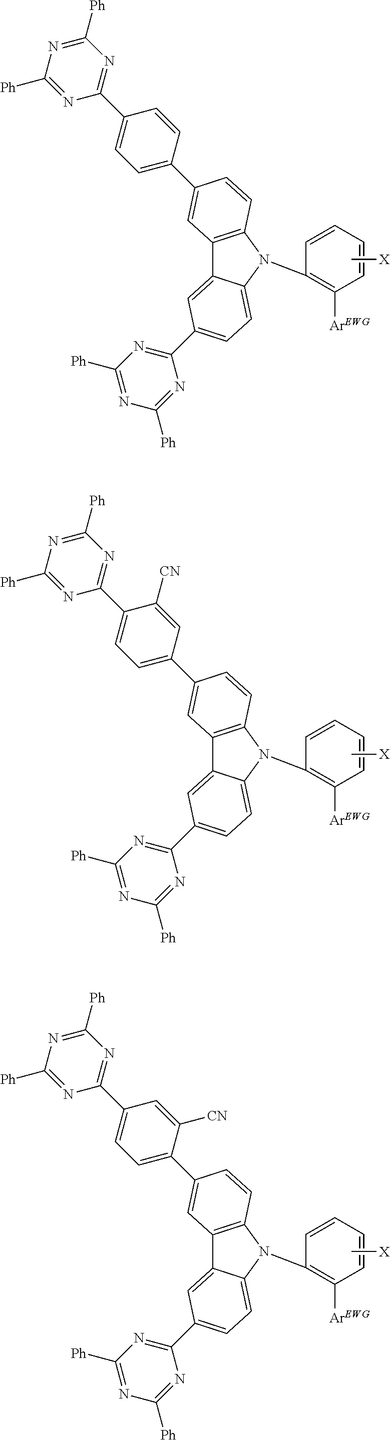

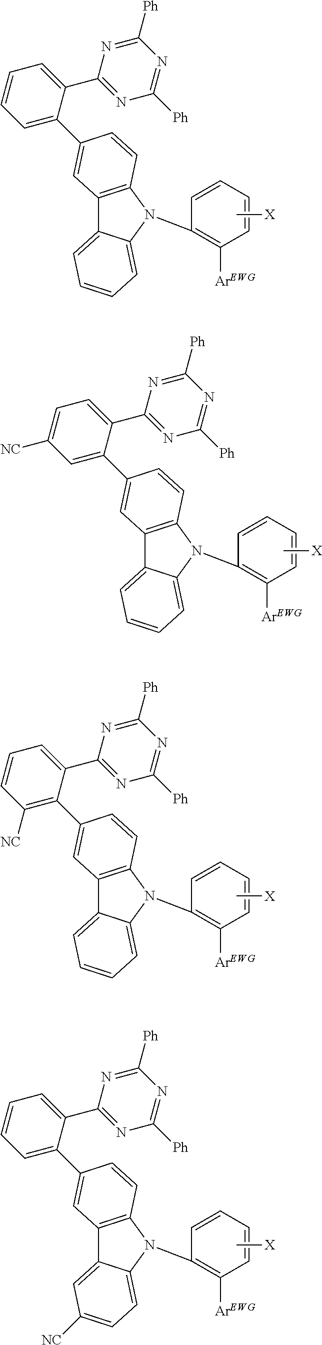

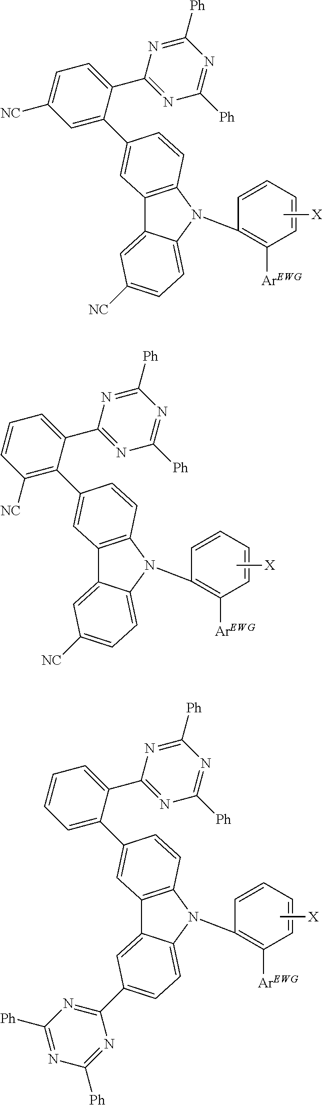

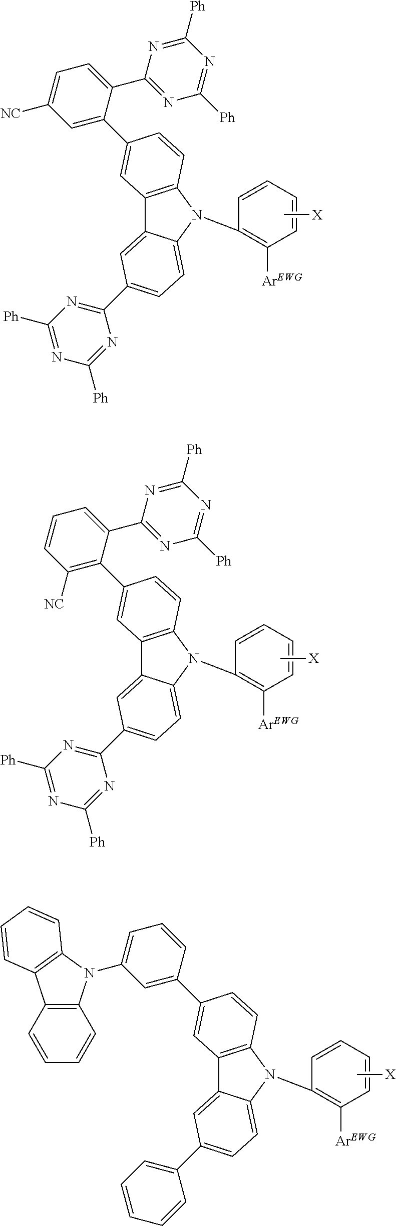

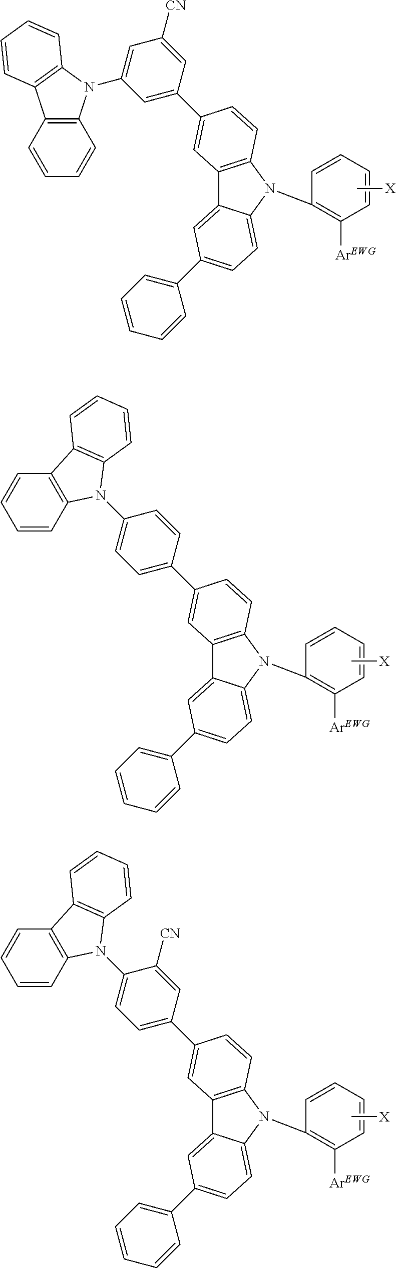

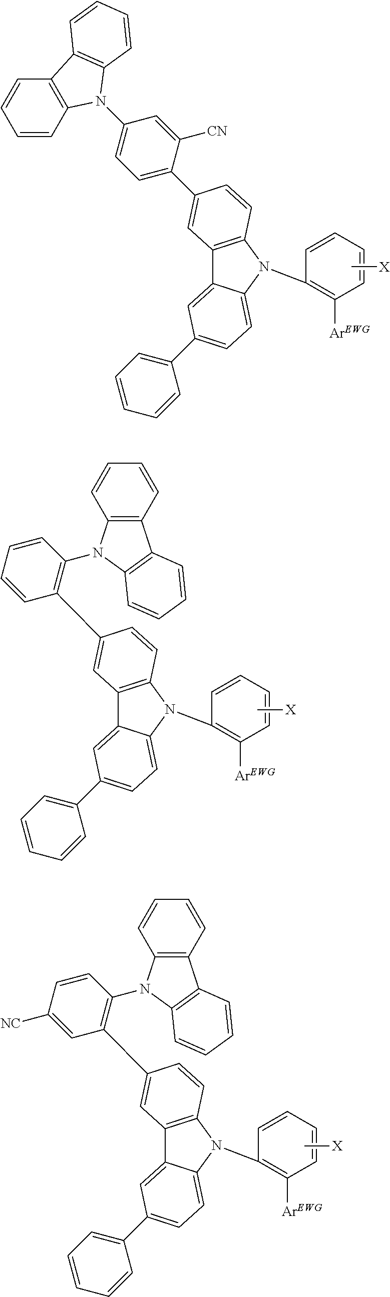

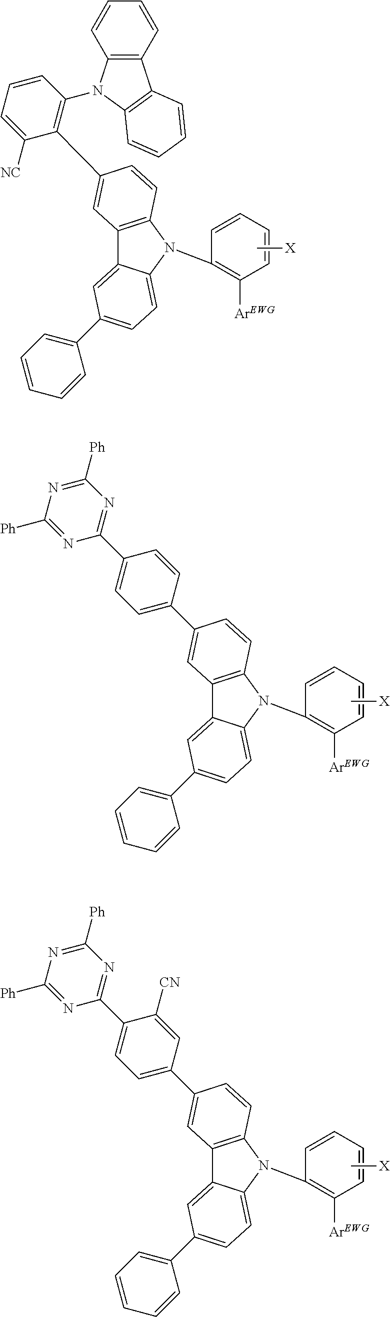

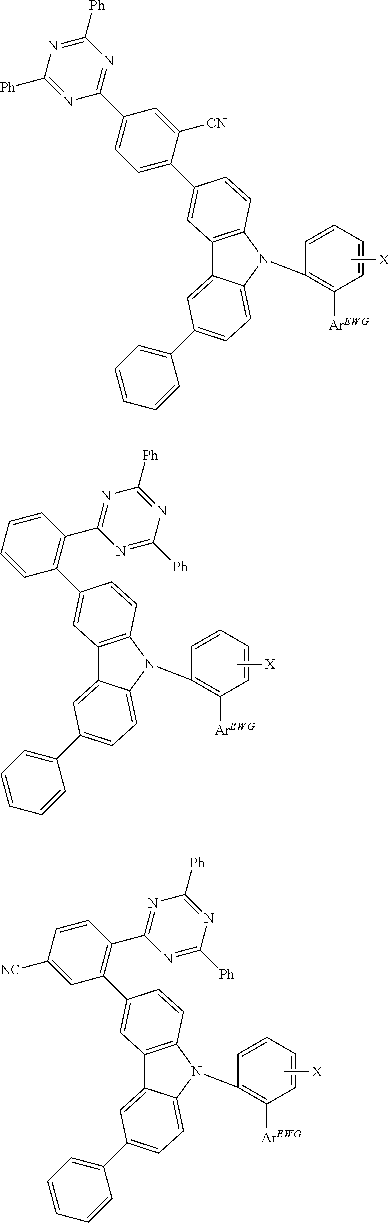

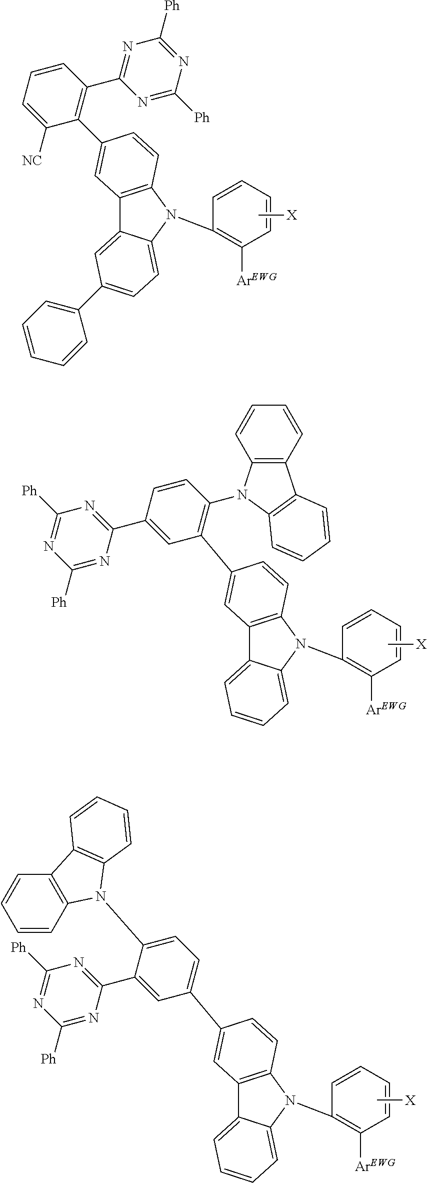

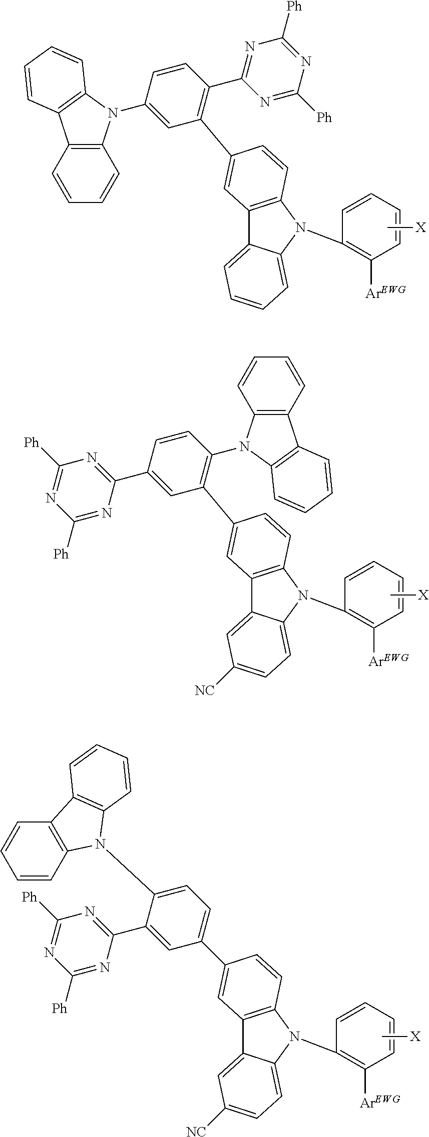

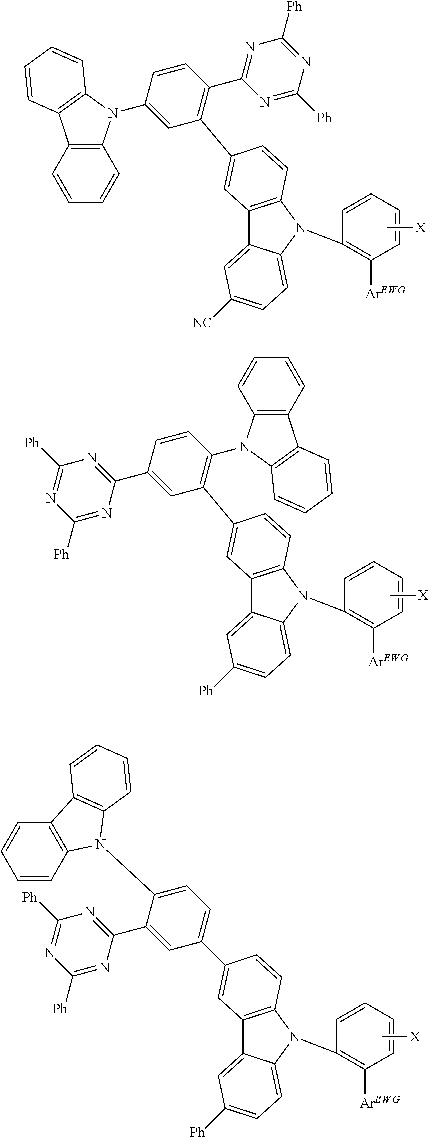

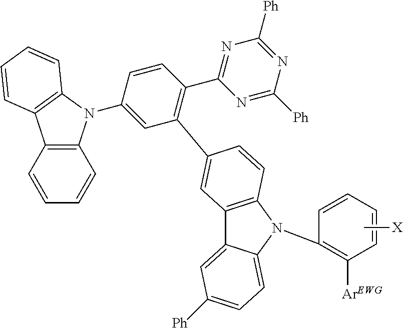

7. The organic molecule according to claim 1, comprising a structure selected from the group consisting of Formula IB and Formula IC:

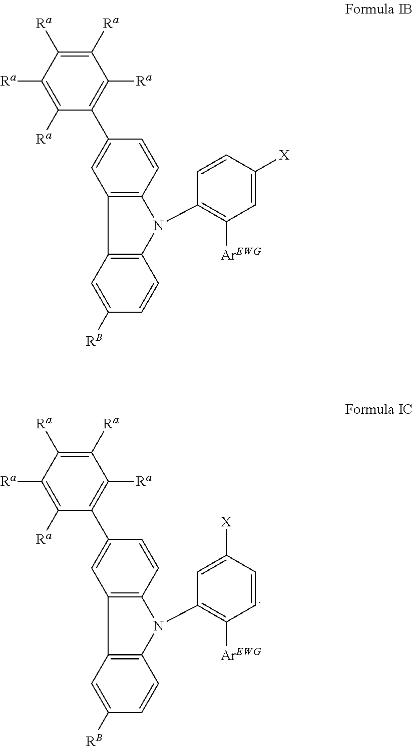

8.-15. (canceled)

16. A composition comprising:
(a) at least one organic molecule according to claim 1 as an emitter and/or a host;
(b) one or more emitter and/or host materials different from the organic molecule according to claim 1, and
(c) optionally one or more dyes and/or one or more solvents.

17. An optoelectronic device comprising the organic molecule according to claim 1.

18. The optoelectronic device according to claim 17, wherein the optoelectronic device is an organic light-emitting diode, a light-emitting electrochemical cell, an organic light-emitting sensor, an organic diode, an organic solar cell, an organic transistor, an organic field-effect transistor, an organic laser or a down-conversion element.

19. The optoelectronic device according to claim 18, comprising:
a substrate;
an anode;
a cathode, wherein the anode or the cathode is disposed on the substrate; and
at least one light-emitting layer disposed between the anode and the cathode and which comprises the organic molecule.

20. An optoelectronic device comprising the organic molecule according to claim 1, wherein the organic molecule is one of a luminescent emitter, an electron transport material, a hole injection material or a hole blocking material in the optoelectronic device.

21. An optoelectronic device comprising the organic molecule according to claim 2, wherein the optoelectronic device is an organic light-emitting diode, a light-emitting electrochemical cell, an organic light-emitting sensor, an organic diode, an organic solar cell, an organic transistor, an organic field-effect transistor, an organic laser or a down-conversion element.

22. The optoelectronic device according to claim 21, comprising:
a substrate;
an anode;
a cathode, wherein the anode or the cathode is applied to the substrate; and
at least one light-emitting layer disposed between the anode and the cathode and which comprises the organic molecule.

23. An optoelectronic device comprising the organic molecule according to claim 3, wherein the optoelectronic device is an organic light-emitting diode, a light-emitting electrochemical cell, an organic light-emitting sensor, an organic diode, an organic solar cell, an organic transistor, an organic field-effect transistor, an organic laser or a down-conversion element.

24. The optoelectronic device according to claim 23, comprising:
a substrate;
an anode;
a cathode, wherein the anode or the cathode is applied to the substrate; and
at least one light-emitting layer disposed between the anode and the cathode and which comprises the organic molecule.

25. An optoelectronic device comprising the composition according to claim 16.

26. The optoelectronic device according to claim 25, wherein the optoelectronic device is an organic light-emitting diode, a light-emitting electrochemical cell, an organic light-emitting sensor, an organic diode, an organic solar cell, an organic transistor, an organic field-effect transistor, an organic laser or a down-conversion element.

27. The optoelectronic device according to claim 26, comprising:
a substrate;
an anode;
a cathode, wherein the anode or the cathode is disposed on the substrate; and
at least one light-emitting layer disposed between the anode and the cathode and which comprises the composition.

28. A process for producing an optoelectronic device, comprising processing of the organic molecule according to claim 1 by a vacuum evaporation method or from a solution.
